(19) United States
(12) Patent Application Publication
(10) Pub. No.: US 2025/0051250 A1
(43) Pub. Date: Feb. 13, 2025

(54) DIAMANTANE COMPOUND, ORGANIC ELECTROLUMINESCENT DEVICE MATERIAL AND ORGANIC ELECTROLUMINESCENT DEVICE

(71) Applicants: TOSOH CORPORATION, Yamaguchi (JP); SAGAMI CHEMICAL RESEARCH INSTITUTE, Kanagawa (JP)

(72) Inventors: Fuminari UEHARA, Kanagawa (JP); Naoki UCHIDA, Kanagawa (JP); Kana OIKE, Kanagawa (JP); Yasuhiro TAKAHASHI, Kanagawa (JP); Kazuki HATTORI, Kanagawa (JP); Shintaro NOMURA, Kanagawa (JP); Hidenori AIHARA, Kanagawa (JP); Naoki HAYAKAWA, Kanagawa (JP); Shuya MATSUMOTO, Kanagawa (JP)

(73) Assignees: TOSOH CORPORATION, Yamaguchi (JP); SAGAMI CHEMICAL RESEARCH INSTITUTE, Kanagawa (JP)

(21) Appl. No.: 18/721,316

(22) PCT Filed: Dec. 6, 2022
(86) PCT No.: PCT/JP2022/044854

§ 371 Date: Jun. 18, 2024

(30) Foreign Application Priority Data
Dec. 22, 2021 (JP) 2021-208545
May 11, 2022 (JP) 2022-077905

(51) Int. Cl. C07C 15/38; H10K 50/15; H10K 50/16; H10K 85/60

(52) U.S. Cl. CPC C07C 15/38; H10K 50/15; H10K 50/16; H10K 85/624; H10K 85/626

(57) ABSTRACT

Provided are a diamantane compound with which an organic electroluminescent device that can exhibit excellent luminous efficiency characteristics, more preferably excellent drive voltage characteristics and excellent luminous efficiency characteristics, can be produced, and an organic electroluminescent device material containing the diamantane compound. Also provided is an organic electroluminescent device that can achieve luminous efficiency characteristics, more preferably drive voltage characteristics and luminous efficiency characteristics, at high levels.

A diamantane compound has a group represented by formula (1) below and a group represented by formula (2) below.

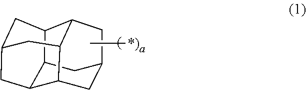

(In formula (1), carbon atoms of a diamantane ring are optionally substituted with an aryl group having 6 to 12 carbon atoms. In formula (1), * represents a bond, and a represents an integer of 1 to 6.)

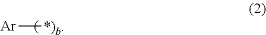

### TECHNICAL FIELD

The present invention relates to a diamantane compound, an organic electroluminescent device material and an organic electroluminescent device.

### BACKGROUND ART

Organic electroluminescent devices have been put into practical use mainly for small-sized mobile applications. However, performance improvement is essential for further expansion of applications, and materials having low drive voltages and high luminous efficiency characteristics are demanded.

PTL 1 discloses an azine compound that is an organic electroluminescent device material capable of achieving a lower drive voltage with high efficiency.

PTL 2 discloses a compound that is an organic electroluminescent device material in which a fluorene moiety and a six-membered heterocycle are bonded through a linker group to form a basic skeleton and which is represented by a specific structural formula.

### CITATION LIST

### Patent Literature

PTL 1: Japanese Unexamined Patent Application Publication No. 2012-82136
PTL 2: Japanese Unexamined Patent Application Publication (Translation of PCT Application) No. 2018-507174

### SUMMARY OF INVENTION

### Technical Problem

However, market demands for expansion of applications and expansion of usable environments are very strong, and there is a need to provide organic electroluminescent devices further excellent in two characteristics, drive voltage and luminous efficiency. From the viewpoint of providing an organic electroluminescent device that exhibits a more excellent drive voltage and more excellent luminous efficiency, the compounds disclosed in PTLs 1 and 2 above, which are organic electroluminescent device materials, are less than sufficient and leave room for improvement.

Accordingly, an aspect of the present invention provides a compound which is an organic electroluminescent device material and with which an organic electroluminescent device that can exhibit excellent drive voltage characteristics and excellent luminous efficiency characteristics can be produced.

Furthermore, another aspect of the present invention provides an organic electroluminescent device that can achieve drive voltage characteristics and luminous efficiency characteristics at high levels.

### Solution to Problem

As a result of intensive studies to solve the above problems, the present inventors have found that use of a specific diamantane compound as an electron transport layer or a hole transport layer of an organic electroluminescent device improves luminous efficiency characteristics, more preferably drive voltage characteristics and luminous efficiency characteristics, of the organic electroluminescent device, thereby completing the present invention.

Thus, the present invention includes the following aspects.

[1]A diamantane compound comprising a group represented by formula (1) below and a group represented by formula (2) below.

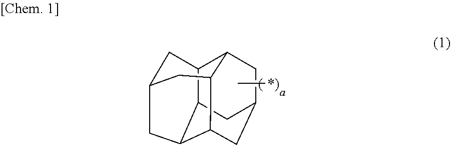

(In formula (1), carbon atoms of a diamantane ring are optionally substituted with an aryl group having 6 to 12 carbon atoms. In formula (1), * represents a bond, and a represents an integer of 1 to 6.)

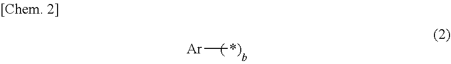

(Ar in formula (2) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
   provided that in Ar, a total number of carbon atoms constituting Ar is 8 or more, and when Ar is constituted only by the aromatic hydrocarbon group having 6 to 60 carbon atoms of (i), the total number of carbon atoms constituting Ar is 13 or more. In formula (2), * represents a bond, and b represents an integer of 1 to 6.)
   [2] The diamantane compound according to [1], wherein the diamantane compound is represented by formula (3) below or formula (4) below.

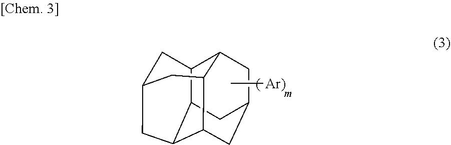

(In a diamantane ring in formula (3), carbon atoms other than a carbon atom bonded to an —Ar group are optionally substituted with an aryl group having 6 to 12 carbon atoms.

Each Ar in formula (3) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
   provided that in each independent Ar in formula (3), a total number of carbon atoms constituting Ar is 8 or more, and when Ar is constituted only by the aromatic hydrocarbon group having 6 to 60 carbon atoms of (i), the total number of carbon atoms constituting Ar is 13 or more.

In formula (3), m represents an integer of 1, 2 or 3.)

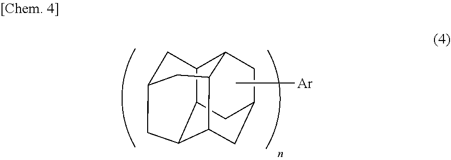

(In each independent diamantane ring in formula (4), carbon atoms other than a carbon atom bonded to an —Ar group are optionally substituted with an aryl group having 6 to 12 carbon atoms.

Ar in formula (4) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
   provided that in Ar in formula (4), a total number of carbon atoms constituting Ar is 8 or more, and when Ar is constituted only by the aromatic hydrocarbon group having 6 to 60 carbon atoms of (i), the total number of carbon atoms constituting Ar is 13 or more.

In formula (4), n represents an integer of 2 or 3.) [3] The diamantane compound according to [2], wherein the diamantane compound is represented by any of formula (3-1) below to formula (3-3) below.

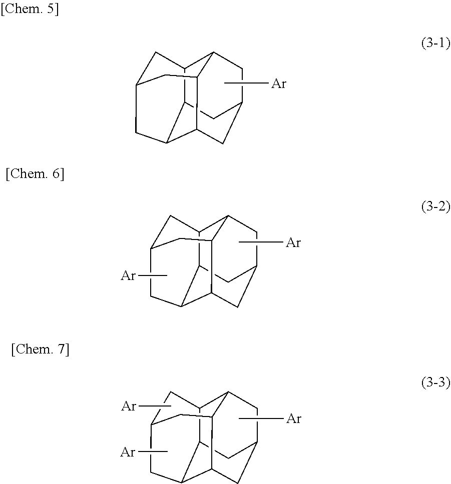

(In diamantane rings in formula (3-1) to formula (3-3), carbon atoms other than a carbon atom bonded to an —Ar group are optionally substituted with an aryl group having 6 to 12 carbon atoms.

Each Ar in formula (3-1) to formula (3-3) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
   provided that in each independent Ar in formula (3-1) to formula (3-3), a total number of carbon atoms constituting Ar is 8 or more, and when Ar is constituted only by the aromatic hydrocarbon group having 6 to 60 carbon atoms of (i), the total number of carbon atoms constituting Ar is 13 or more.)
   [4] The diamantane compound according to [2], wherein the diamantane compound is represented by formula (4-1) below or formula (4-2) below.

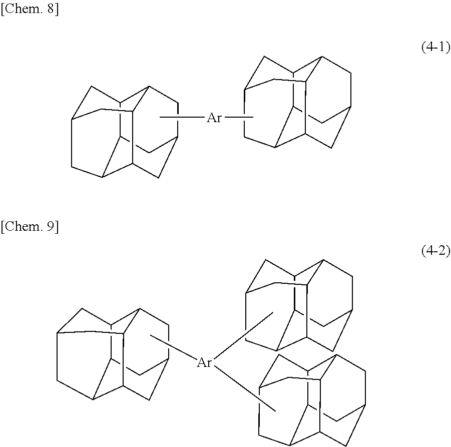

(In each independent diamantane ring in formula (4-1) and formula (4-2), carbon atoms other than a carbon atom bonded to an —Ar group are optionally substituted with an aryl group having 6 to 12 carbon atoms.

Ar in formula (4-1) and formula (4-2) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
   provided that in Ar in formula (4-1) and formula (4-2), a total number of carbon atoms constituting Ar is 8 or more, and when Ar is constituted only by the aromatic hydrocarbon group having 6 to 60 carbon atoms of (i), the total number of carbon atoms constituting Ar is 13 or more.
   [5] The diamantane compound according to any one of [1] to [4], wherein the heteroaromatic group is a monocyclic ring or fused ring including any of a nitrogen atom, an oxygen atom and a sulfur atom, or a linked ring formed by linkage of any rings selected from such rings.
   [6] The diamantane compound according to any one of [1] to [5], wherein the aromatic hydrocarbon group includes an arylamino group, or
the heteroaromatic group includes a heterocyclic amino group.
   [7] The diamantane compound according to any one of [1] to [6], wherein a substituent in the optionally substituted aromatic hydrocarbon group of (i) or the optionally substituted heteroaromatic group of (ii) is a group selected from the group consisting of a fluorine atom, a chlorine atom, a bromine atom, an iodine atom, a formyl group, a cyano group, a nitro group, alkyl groups having 1 to 20 carbon atoms, alkenyl groups having 1 to 20 carbon atoms, cycloalkyl groups having 1 to 20 carbon atoms, bicycloalkyl groups having 1 to 20 carbon atoms, tricycloalkyl groups having 1 to 20 carbon atoms, alkoxy groups having 1 to 10 carbon atoms, P(═O) (Ar²)₂, C(═O) Ar², B(Ar²)₂, B(OAr²)₂, OSO₂Ar², S(═O) Ar², Si(Ar²)₃, P(═S) (Ar²)₂ and C(═C(CN)₂) Ar²₂, (where Ar² is a group selected from the group consisting of alkyl groups having 1 to 10 carbon atoms, aryl groups having 6 to 60 carbon atoms and heteroaromatic groups having 3 to 60 carbon atoms).
   [8] The diamantane compound according to [5], wherein the diamantane compound has the optionally substituted heteroaromatic group having 3 to 60 carbon atoms of (ii), the heteroaromatic group including any of a nitrogen atom, an oxygen atom and a sulfur atom.
   [9] The diamantane compound according to [8], wherein the diamantane compound has the optionally substituted heteroaromatic group having 3 to 60 carbon atoms of (ii), the heteroaromatic group including a nitrogen atom.
   [10] The diamantane compound according to any one of [1] to [9], wherein the diamantane compound has a fused-ring aromatic hydrocarbon group having 14 or more carbon atoms.
   [11] The diamantane compound according to any one of [1] to [10], wherein in the diamantane ring, carbon atoms other than a carbon atom bonded to an —Ar group are not substituted with an aryl group.
   [12] An organic electroluminescent device material comprising diamantane compound according to any one of [1] to [11].
   [13] The organic electroluminescent device material according to [12], wherein the organic electroluminescent device material is an electron transport material for an organic electroluminescent device or a hole transport material for an organic electroluminescent device.
   [14] An organic electroluminescent device comprising the diamantane compound according to any one of [1] to [11].
   [15]A diamantane compound represented by formula (5) below or formula (6) below.

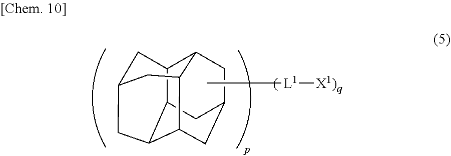

(In a diamantane ring in formula (5), carbon atoms other than a carbon atom bonded to an -L¹-X¹ group are optionally substituted with an aryl group having 6 to 12 carbon atoms.

Each L¹ in formula (5) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

A total number of carbon atoms constituting L¹ is 8 or more.

X¹ in formula (5) represents a halogen atom or a trifluoromethanesulfonyloxy group.

In formula (5), p and q each represent an integer of 1 to 3, when p is 1, q represents an integer of 1, 2 or 3, and when q is 1, p represents an integer of 1, 2 or 3.)

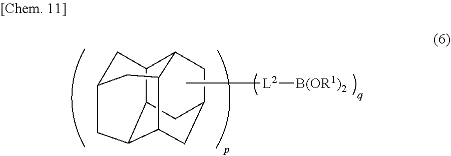

(In a diamantane ring in formula (6), carbon atoms other than a carbon atom bonded to an -L²-B(OR¹)₂ group are optionally substituted with an aryl group having 6 to 12 carbon atoms.

Each L² in formula (6) independently represents a single bond or
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

R¹ in formula (6) represents a hydrogen atom, an alkyl group having 1 to 4 carbon atoms or a phenyl group. Two R¹'s of B(OR¹)₂ may be the same or different. The two R¹'s may be combined to form a ring including an oxygen atom and a boron atom.

In formula (6), p and q each represent an integer of 1 to 3, when p is 1, q represents an integer of 1, 2 or 3, and when q is 1, p represents an integer of 1, 2 or 3.)

### Advantageous Effects of Invention

An aspect of the present invention can provide a diamantane compound with which an organic electroluminescent device that can exhibit excellent luminous efficiency characteristics, more preferably excellent drive voltage characteristics and excellent luminous efficiency characteristics, can be produced, and an organic electroluminescent device material comprising the diamantane compound.

Another aspect of the present invention can provide an organic electroluminescent device that can achieve luminous efficiency characteristics, more preferably drive voltage characteristics and luminous efficiency characteristics, at high levels.

### BRIEF DESCRIPTION OF DRAWING

FIG. 1 A schematic sectional view showing an example of a layered structure of an organic electroluminescent device according to an aspect of the present invention.

FIG. 2 A schematic sectional view showing another example (the configuration in Device Example-1) of a layered structure of an organic electroluminescent device according to an aspect of the present invention.

### DESCRIPTION OF EMBODIMENTS

Hereinafter, a diamantane compound according to an aspect of the present invention will be described in detail.

### <Diamantane Compound>

A diamantane compound according to an aspect of the present invention is a diamantane compound comprising a group represented by formula (1) below and a group represented by formula (2) below.

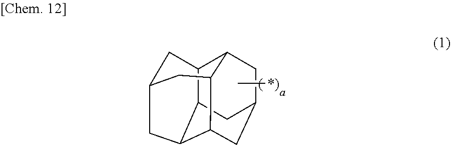

In formula (1), carbon atoms of a diamantane ring are optionally substituted with an aryl group having 6 to 12 carbon atoms. In formula (1), * represents a bond, and a represents an integer of 1 to 6.

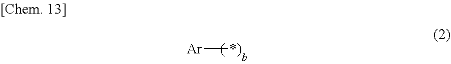

Ar represented in formula (2) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

In Ar represented in formula (2), the total number of carbon atoms constituting Ar is 8 or more, and when Ar is constituted only by the aromatic hydrocarbon group having 6 to 60 carbon atoms of (i), the total number of carbon atoms constituting Ar is 13 or more. In formula (2), * represents a bond, and b represents an integer of 1 to 6.

### [Diamantane Ring in Formula (1)]

In the diamantane ring in formula (1), there is no limitation on a carbon atom bonded to the Ar group represented in formula (2), and the Ar group represented in formula (2) may be bonded to any carbon atom in the diamantane skeleton structure represented by formula (1). In the diamantane ring in formula (1), the number of carbon atoms bonded to the Ar group represented in formula (2) is not particularly limited, and a plurality of carbon atoms of the diamantane ring in formula (1) may be bonded to the Ar group represented in formula (2).

As described above, in the diamantane ring in formula (1), carbon atoms other than a carbon atom bonded to the Ar group represented in formula (2) are optionally substituted with an aryl group having 6 to 12 carbon atoms. However, in the present invention, from the viewpoint of, for example, availability and ease of production, it is more preferable that in the diamantane ring in formula (1), carbon atoms other than a carbon atom bonded to the Ar group not be substituted with an aryl group.

Examples of the aryl group as a substituent include a phenyl group, a naphthyl group and a biphenyl group.

### [Regarding Ar in Formula (2)]

Ar represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

The aromatic hydrocarbon group and the heteroaromatic group may each be a monocyclic ring or fused ring or a linked ring formed by linkage of any rings selected from such rings.

Examples of the aromatic hydrocarbon group having 6 to 60 carbon atoms include a phenyl group, a naphthyl group, a phenanthryl group, an antonyl group, a pyrenyl group, a perylenyl group, a fluorenyl group, a dimethylfluorenyl group, a diphenylfluorenyl group, a triphenylenyl group, a fluoranthenyl group, a benzofluoranthenyl group, a chrysenyl group, a dibenzochrysenyl group and a dinaphthochrysenyl group.

In the present invention, aromatic hydrocarbon groups include arylamino groups. An arylamino group refers to a group derived by substituting one or both hydrogen atoms of an amino group with an aryl group. Examples of arylamino groups include a phenylamino group, a diphenylamino group, a biphenylylamino group, a dibiphenylylamino group, a naphthylamino group and a dinaphthylamino group.

In the present invention, the aromatic hydrocarbon group having 6 to 60 carbon atoms is more preferably a fused-ring aromatic hydrocarbon group having 14 or more carbon atoms.

The heteroaromatic group is preferably, for example, a heteroaromatic group including any of a nitrogen atom, an oxygen atom and a sulfur atom. In particular, the heteroaromatic group is preferably a heteroaromatic group including a nitrogen atom.

Examples of the heteroaromatic group having 3 to 60 carbon atoms include a pyridyl group, a pyrimidyl group, a pyrazyl group, a triazinyl group, a quinolyl group, an isoquinolyl group, a nadithyridinyl group, an acridinyl group, a phenanthrolinyl group, a phenanthridinyl group, a pyrrolyl group, an indolyl group, an indolizinyl group, a carbazolyl group, a carbolinyl group, a benzocarbazolyl group, a benzocarbolinyl group, a furanyl group, a benzofuranyl group, a dibenzofuranyl group, a xanthenyl group, a spiroxanthenyl group, a benzoxanthenyl group, a thienyl group, a benzothienyl group, a dibenzothienyl group, an oxazolyl group, a benzooxazolyl group, a thiazolyl group and a benzothiazalyl group.

In the present invention, heteroaromatic groups include heterocyclic amino groups. A heterocyclic amino group refers to a group derived by substituting one or both hydrogen atoms of an amino group with a heteroaromatic group. Examples of heterocyclic amino groups include a pyridylamino group, a dipyridylamino group, a pyrimidylamino group, a dipyrimidylamino group, a pyrazylamino group and a dipyrazylamino group.

The substituent in the optionally substituted aromatic hydrocarbon group of (i) above or the optionally substituted heteroaromatic group of (ii) above may be a group selected from the group consisting of a fluorine atom, a chlorine atom, a bromine atom, an iodine atom, a formyl group, a cyano group, a nitro group, alkyl groups having 1 to 20 carbon atoms, alkenyl groups having 1 to 20 carbon atoms, cycloalkyl groups having 1 to 20 carbon atoms, bicycloalkyl groups having 1 to 20 carbon atoms, tricycloalkyl groups having 1 to 20 carbon atoms, alkoxy groups having 1 to 10 carbon atoms, P(═O) (Ar²)₂, C(═O)Ar², B(Ar²)₂, B(OAr²)₂, OSO₂Ar², S(═O) Ar², Si(Ar²)₃, P(═S) (Ar²)₂ and C(═C(CN)₂) Ar²₂.

Here, Ar² may be a group selected from the group consisting of alkyl groups having 1 to 10 carbon atoms, aryl groups having 6 to 60 carbon atoms and heteroaromatic groups having 3 to 60 carbon atoms.

Ar in formula (2) above preferably has a group selected from the group consisting of a phenyl group, a naphthyl group, an azabenzofluoranthenyl group, a triazinyl group, a fluoranthenyl group, a benzofluoranthenyl group and a triazinylphenyl group.

In a preferred exemplary embodiment, the diamantane compound according to the present invention comprising the group represented by formula (1) above and the group represented by formula (2) above is a diamantane compound according to the following first embodiment or a diamantane compound according to the following second embodiment. The diamantane compound according to the first embodiment and the diamantane compound according to the second embodiment will each be described below.

### First Embodiment

In a preferred exemplary embodiment, the diamantane compound according to the present invention is a diamantane compound represented by formula (3) below.

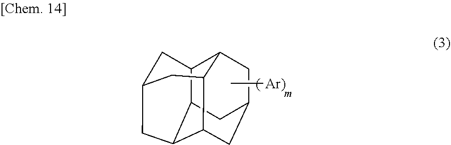

In a diamantane ring in formula (3), carbon atoms other than a carbon atom bonded to an Ar group are optionally substituted with an aryl group having 6 to 12 carbon atoms.

Each Ar in formula (3) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

In each independent Ar in formula (3), the total number of carbon atoms constituting Ar is 8 or more, and when Ar is constituted only by the aromatic hydrocarbon group having 6 to 60 carbon atoms of (i), the total number of carbon atoms constituting Ar is 13 or more.

In formula (3), m represents an integer of 1, 2 or 3.

Furthermore, in a preferred exemplary embodiment, the diamantane compound represented by formula (3) above is a diamantane compound represented by any of formula (3-1) below to formula (3-3) below.

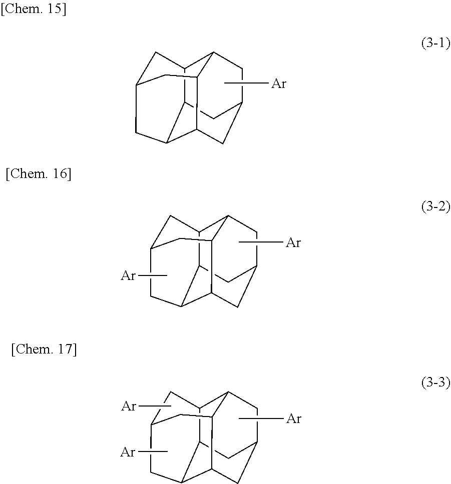

In diamantane rings in formula (3-1) to formula (3-3), carbon atoms other than a carbon atom bonded to an —Ar group are optionally substituted with an aryl group having 6 to 12 carbon atoms.

Each Ar in formula (3-1) to formula (3-3) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

In each independent Ar in formula (3-1) to formula (3-3), the total number of carbon atoms constituting Ar is 8 or more, and when Ar is constituted only by the aromatic hydrocarbon group having 6 to 60 carbon atoms of (i), the total number of carbon atoms constituting Ar is 13 or more.

In formula (3) above and formulae (3-1) to (3-3) above, descriptions on diamantyl groups and Ar groups are the same as given in the section of [Diamantane Ring in Formula (1)] above and the section of [Regarding Ar in Formula (2)] above.

### Second Embodiment

In a preferred exemplary embodiment, the diamantane compound according to the present invention is a diamantane compound represented by formula (4) below.

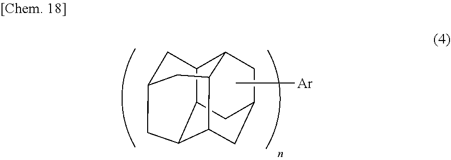

In each independent diamantane ring in formula (4), carbon atoms other than a carbon atom bonded to an —Ar group are optionally substituted with an aryl group having 6 to 12 carbon atoms.

Ar in formula (4) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

In Ar in formula (4), the total number of carbon atoms constituting Ar is 8 or more, and when Ar is constituted only by the aromatic hydrocarbon group having 6 to 60 carbon atoms of (i), the total number of carbon atoms constituting Ar is 13 or more.

In formula (4), n represents an integer of 2 or 3.

Furthermore, in a preferred exemplary embodiment, the diamantane compound represented by formula (4) above is a diamantane compound represented by formula (4-1) below or formula (4-2) below.

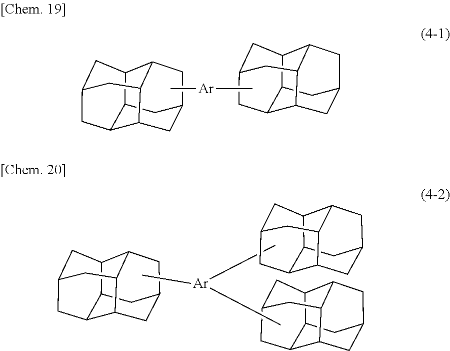

In each independent diamantane ring in formula (4-1) and formula (4-2), carbon atoms other than a carbon atom bonded to an —Ar group are optionally substituted with an aryl group having 6 to 12 carbon atoms.

Ar in formula (4-1) and formula (4-2) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

In Ar in formula (4-1) and formula (4-2), the total number of carbon atoms constituting Ar is 8 or more, and when Ar is constituted only by the aromatic hydrocarbon group having 6 to 60 carbon atoms of (i), the total number of carbon atoms constituting Ar is 13 or more.

In formula (4) above and formulae (4-1) and (4-2) above, descriptions on diamantyl groups and Ar groups are the same as given in the section of [Diamantane Ring in Formula (1)] above and the section of [Regarding Ar in Formula (2)] above.

The molecular weight of the diamantane compound according to the present invention determined by mass spectrometry is preferably 300 to 1200, more preferably 400 to 900. A diamantane compound having a molecular weight in this range has a high glass transition point and is highly stable even if it is repeatedly subjected to electrical oxidation and reduction. Thus, using the diamantane compound according to the present invention, which has a desired molecular weight, as an organic electroluminescent device material can provide an organic electroluminescent device that can achieve drive voltage characteristics and luminous efficiency characteristics at even higher levels.

### [Specific Examples of Diamantane Compound]

Specific examples of the diamantane compound are shown below, but the present invention is not limited thereto.

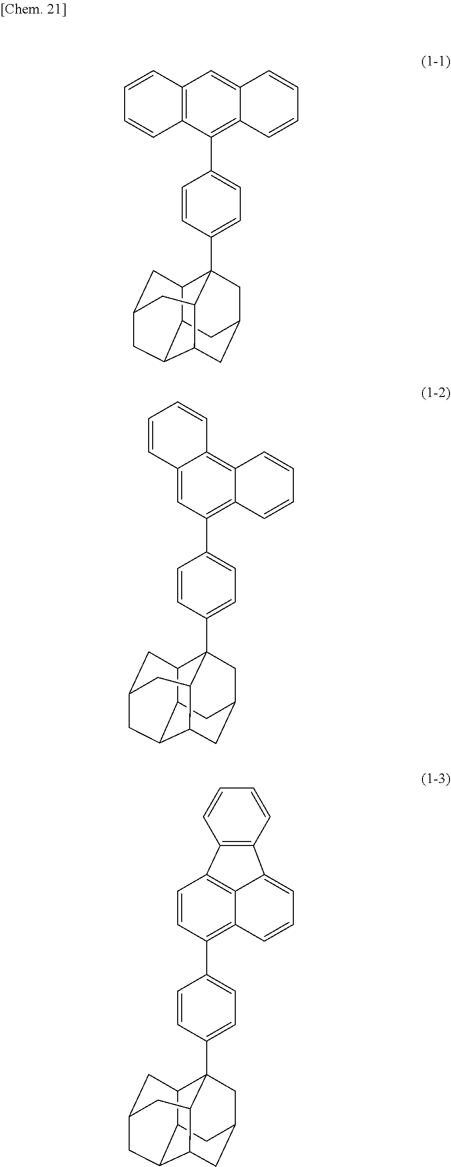

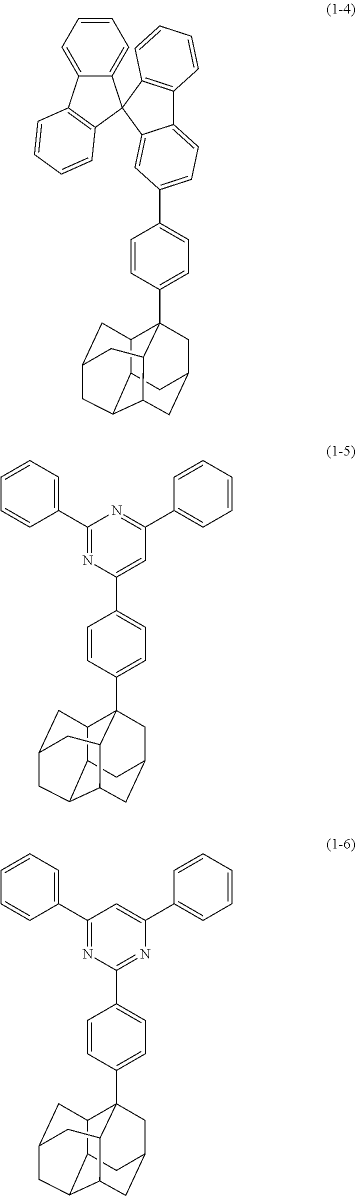

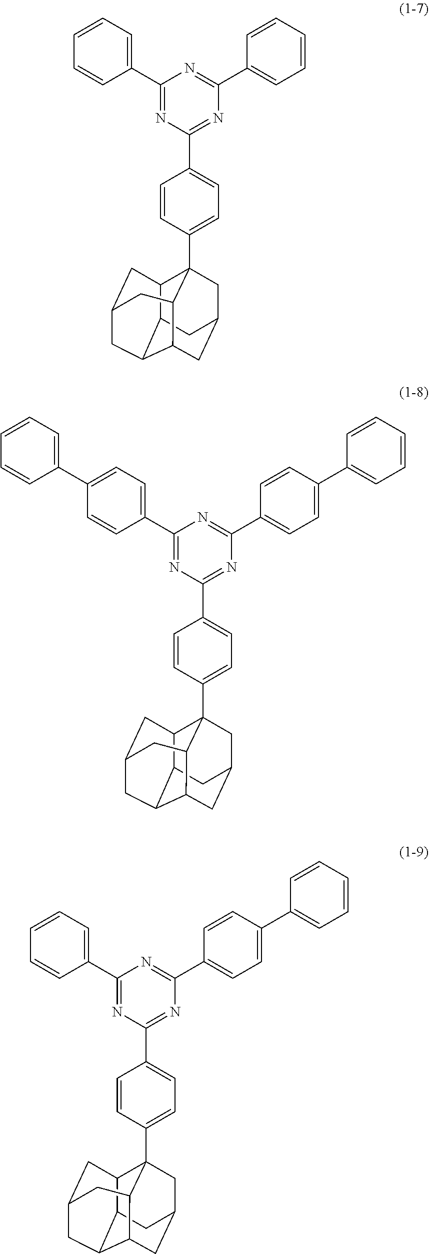 [SMILES: c1ccc(-c2nc(-c3ccccc3)nc(-c3ccc(C45CC6CC7C8CC(CC74)CC5C8C6)cc3)n2)cc1 ; c1ccc(-c2ccc(-c3nc(-c4ccc(-c5ccccc5)cc4)nc(-c4ccc(C56CC7CC8C9CC(CC85)CC6C9C7)cc4)n3)cc2)cc1 ; c1ccc(-c2ccc(-c3nc(-c4ccccc4)nc(-c4ccc(C56CC7CC8C9CC(CC85)CC6C9C7)cc4)n3)cc2)cc1]

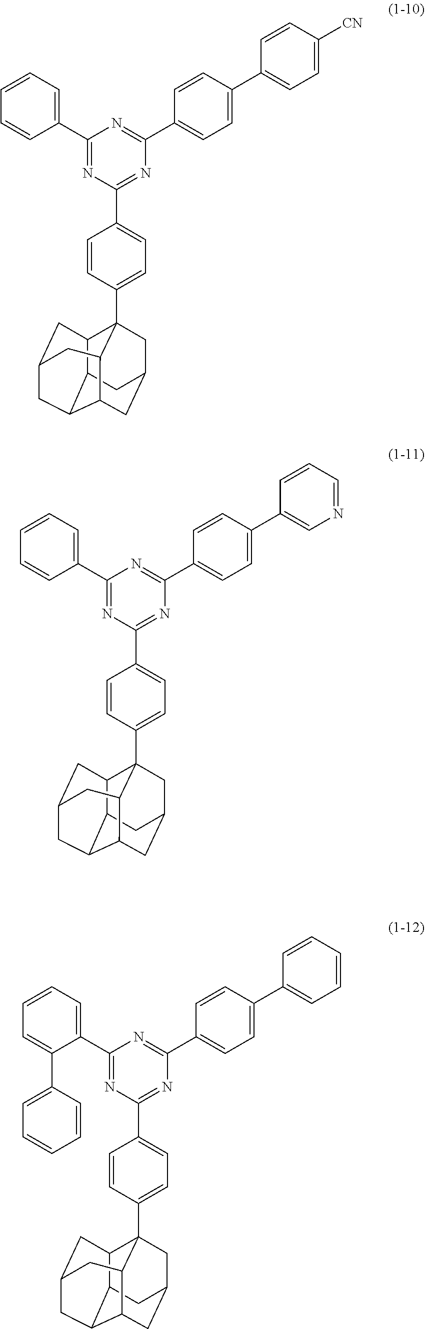 [SMILES: N#Cc1ccc(-c2ccc(-c3nc(-c4ccccc4)nc(-c4ccc(C56CC7CC8C9CC(CC85)CC6C9C7)cc4)n3)cc2)cc1 ; c1ccc(-c2nc(-c3ccc(-c4cccnc4)cc3)nc(-c3ccc(C45CC6CC7C8CC(CC74)CC5C8C6)cc3)n2)cc1 ; c1ccc(-c2ccc(-c3nc(-c4ccc(C56CC7CC8C9CC(CC85)CC6C9C7)cc4)nc(-c4ccccc4-c4ccccc4)n3)cc2)cc1]

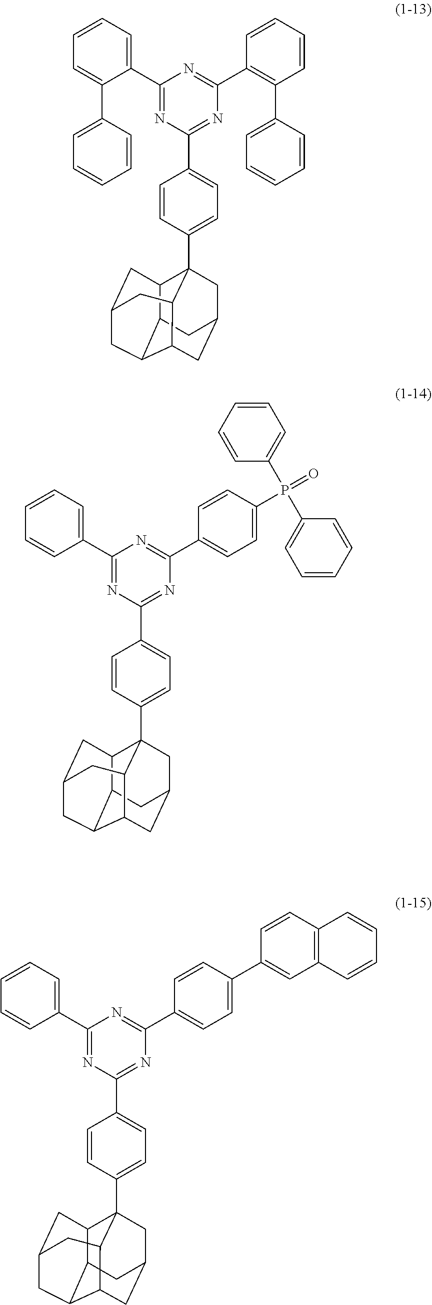 [SMILES: c1ccc(-c2ccccc2-c2nc(-c3ccc(C45CC6CC7C8CC(CC74)CC5C8C6)cc3)nc(-c3ccccc3-c3ccccc3)n2)cc1 ; O=P(c1ccccc1)(c1ccccc1)c1ccc(-c2nc(-c3ccccc3)nc(-c3ccc(C45CC6CC7C8CC(CC74)CC5C8C6)cc3)n2)cc1 ; c1ccc(-c2nc(-c3ccc(-c4ccc5ccccc5c4)cc3)nc(-c3ccc(C45CC6CC7C8CC(CC74)CC5C8C6)cc3)n2)cc1]

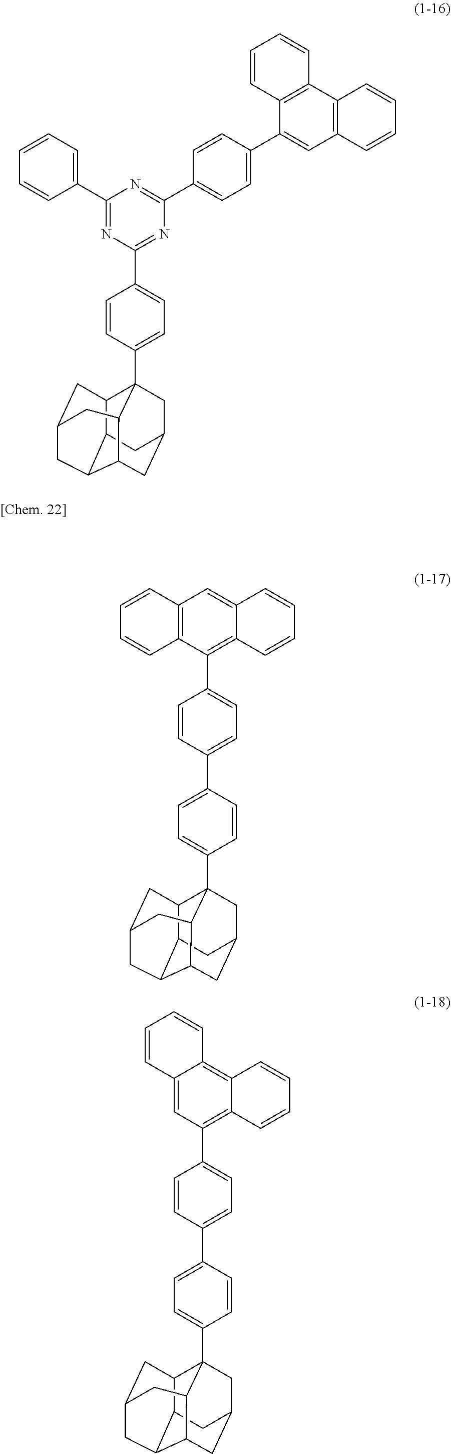

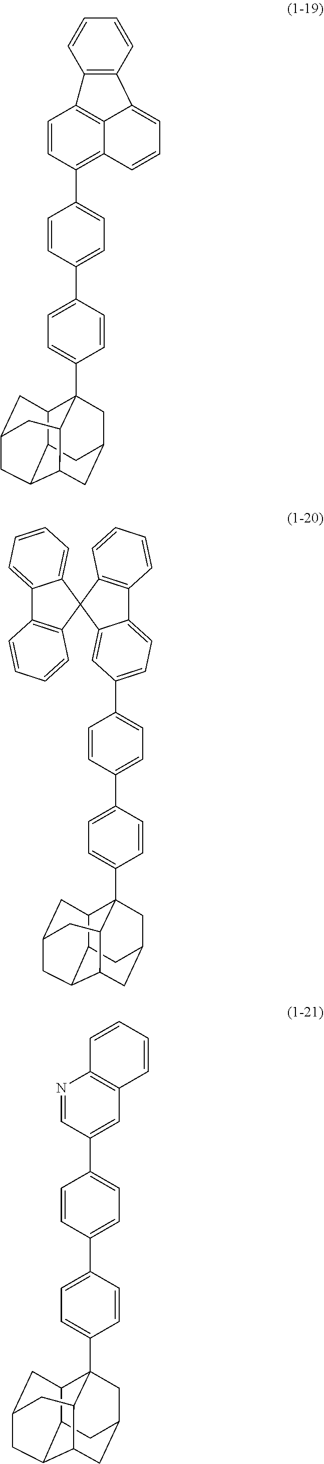

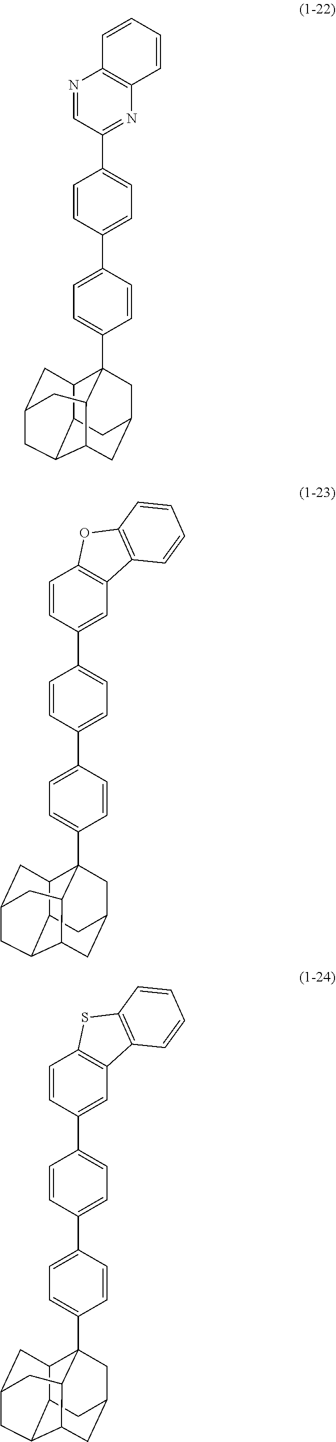 [SMILES: c1ccc2nc(-c3ccc(-c4ccc(C56CC7CC8C9CC(CC85)CC6C9C7)cc4)cc3)cnc2c1 ; c1ccc2c(c1)oc1ccc(-c3ccc(-c4ccc(C56CC7CC8C9CC(CC85)CC6C9C7)cc4)cc3)cc12 ; c1ccc2c(c1)sc1ccc(-c3ccc(-c4ccc(C56CC7CC8C9CC(CC85)CC6C9C7)cc4)cc3)cc12]

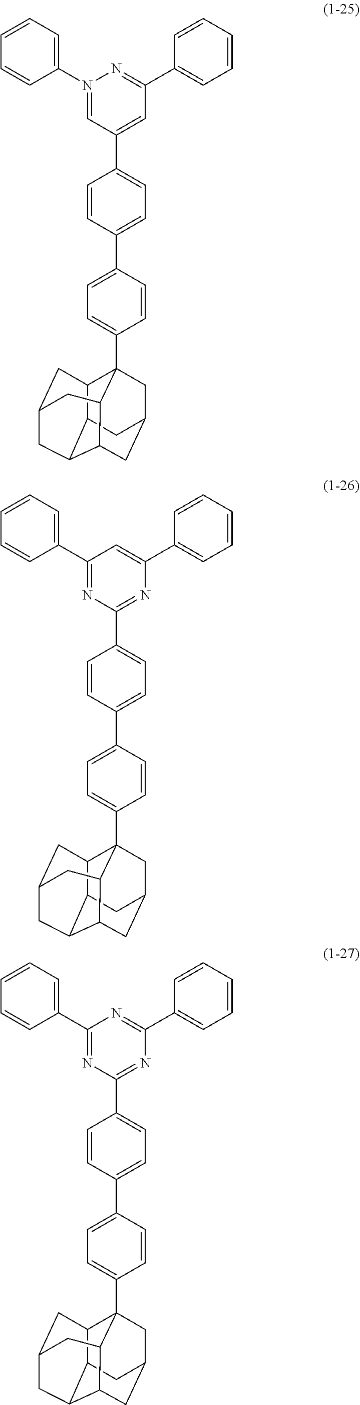

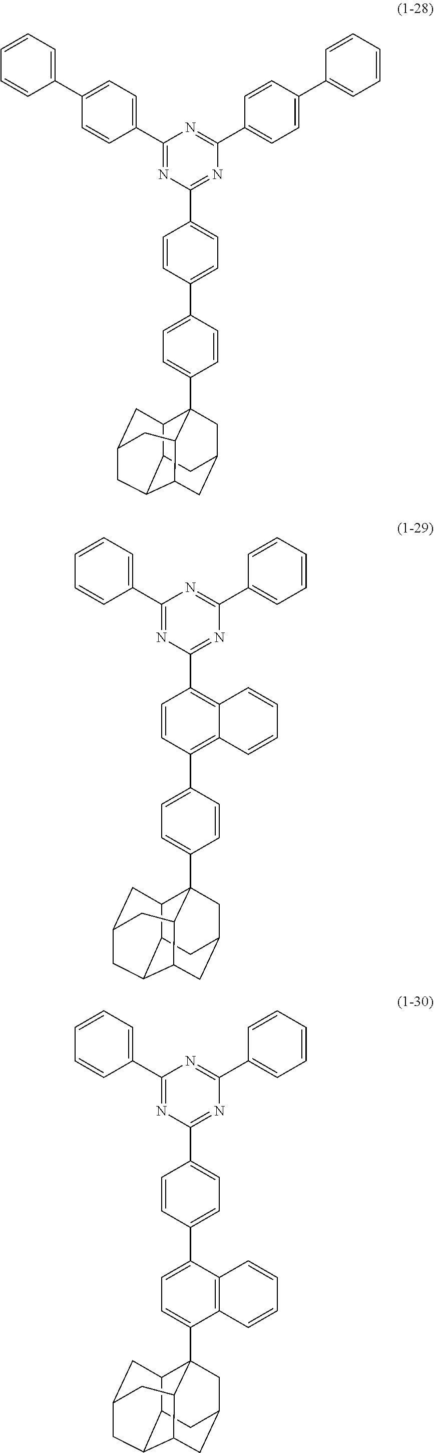 [SMILES: c1ccc(-c2ccc(-c3nc(-c4ccc(-c5ccccc5)cc4)nc(-c4ccc(-c5ccc(C67CC8CC9C%10CC(CC96)CC7C%10C8)cc5)cc4)n3)cc2)cc1 ; c1ccc(-c2nc(-c3ccccc3)nc(-c3ccc(-c4ccc(C56CC7CC8C9CC(CC85)CC6C9C7)cc4)c4ccccc34)n2)cc1 ; c1ccc(-c2nc(-c3ccccc3)nc(-c3ccc(-c4ccc(C56CC7CC8C9CC(CC85)CC6C9C7)c5ccccc45)cc3)n2)cc1]

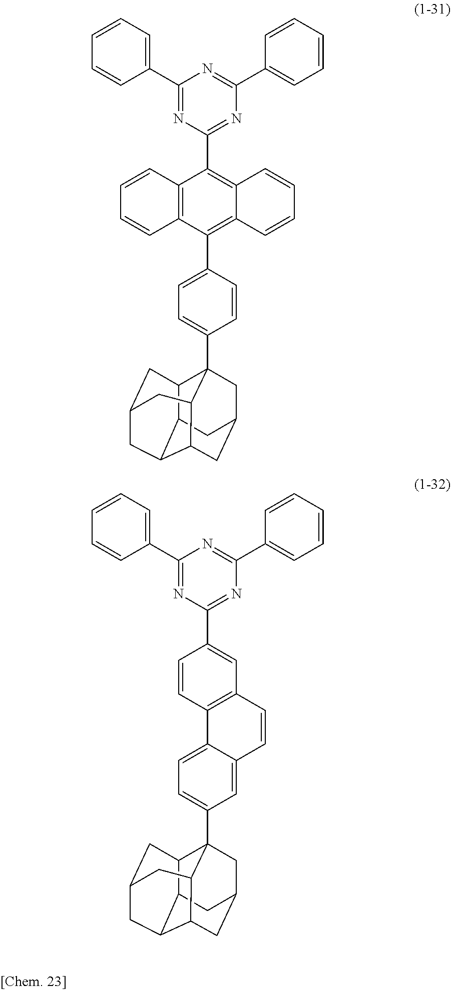 [SMILES: c1ccc(-c2nc(-c3ccccc3)nc(-c3c4ccccc4c(-c4ccc(C56CC7CC8C9CC(CC85)CC6C9C7)cc4)c4ccccc34)n2)cc1 ; c1ccc(-c2nc(-c3ccccc3)nc(-c3ccc4c(ccc5cc(C67CC8CC9C%10CC(CC96)CC7C%10C8)ccc54)c3)n2)cc1]

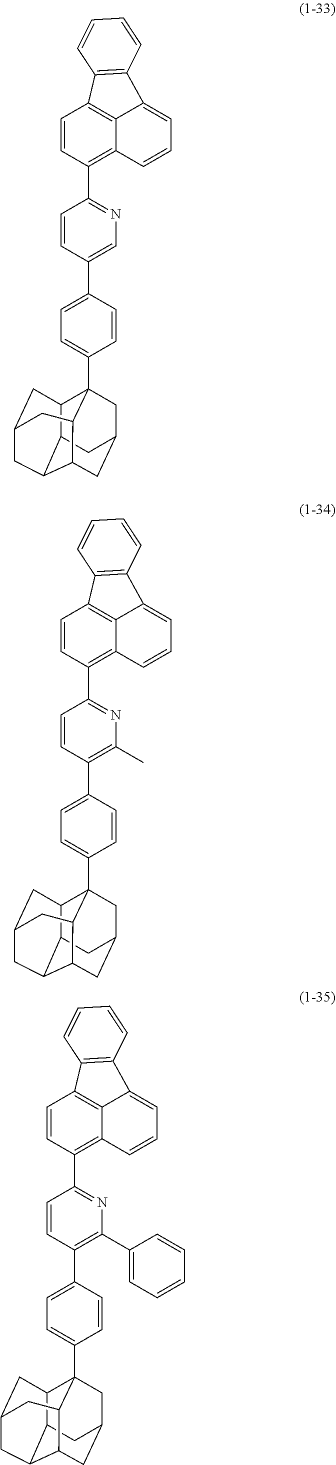

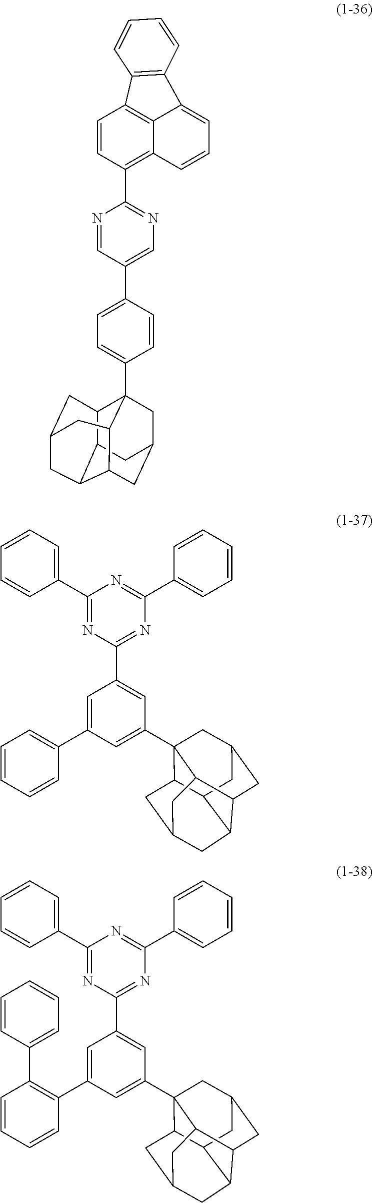

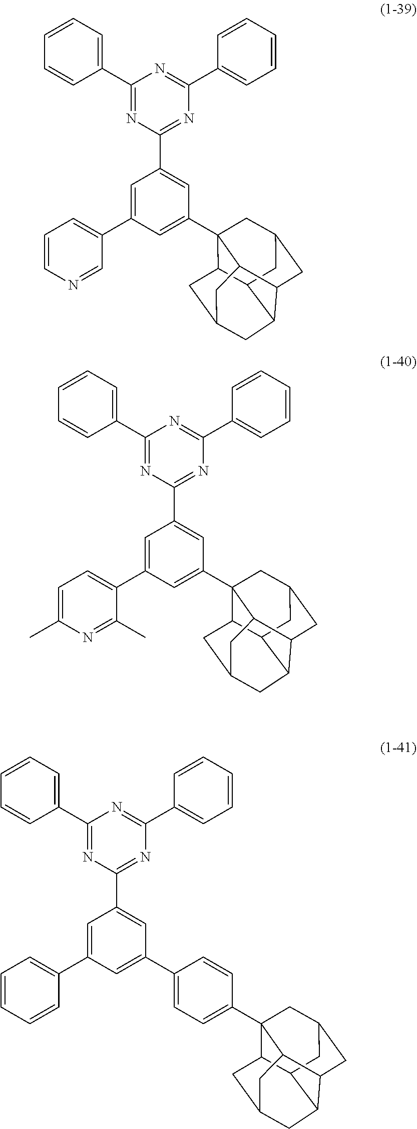

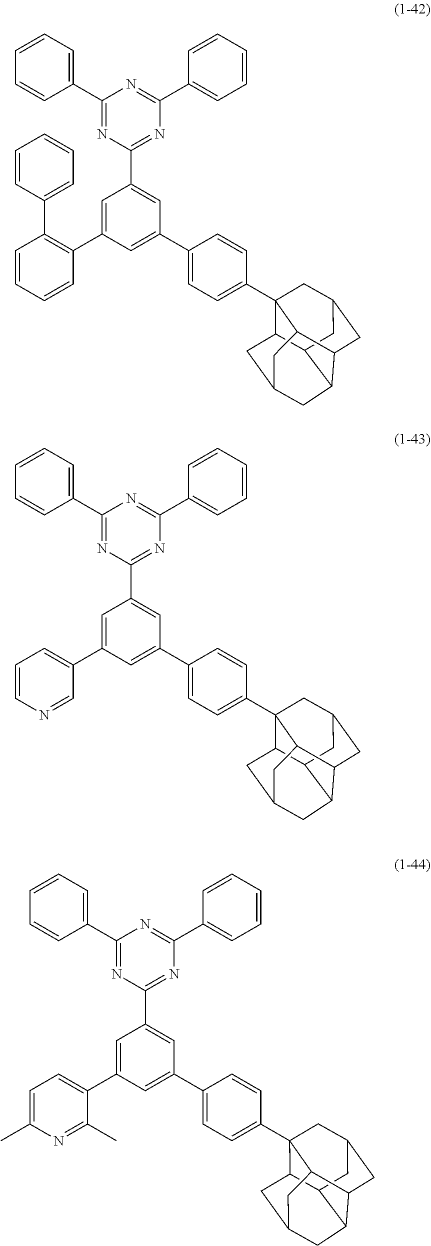 [SMILES: c1ccc(-c2nc(-c3ccccc3)nc(-c3cc(-c4ccc(C56CC7CC8C9CC(CC85)CC6C9C7)cc4)cc(-c4ccccc4-c4ccccc4)c3)n2)cc1 ; c1ccc(-c2nc(-c3ccccc3)nc(-c3cc(-c4ccc(C56CC7CC8C9CC(CC85)CC6C9C7)cc4)cc(-c4cccnc4)c3)n2)cc1 ; Cc1ccc(-c2cc(-c3ccc(C45CC6CC7C8CC(CC74)CC5C8C6)cc3)cc(-c3nc(-c4ccccc4)nc(-c4ccccc4)n3)c2)c(C)n1]

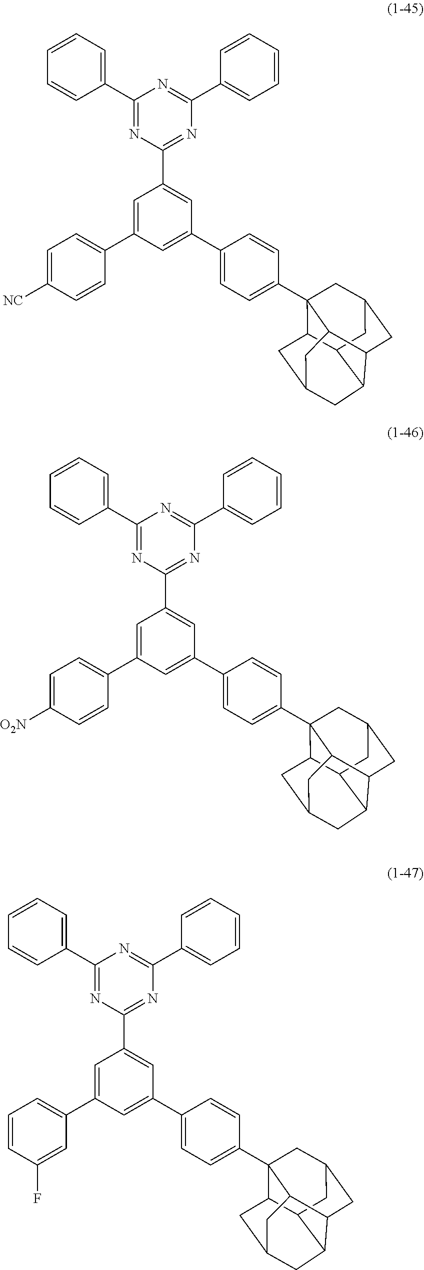 [SMILES: N#Cc1ccc(-c2cc(-c3ccc(C45CC6CC7C8CC(CC74)CC5C8C6)cc3)cc(-c3nc(-c4ccccc4)nc(-c4ccccc4)n3)c2)cc1 ; O=[N+]([O-])c1ccc(-c2cc(-c3ccc(C45CC6CC7C8CC(CC74)CC5C8C6)cc3)cc(-c3nc(-c4ccccc4)nc(-c4ccccc4)n3)c2)cc1 ; Fc1cccc(-c2cc(-c3ccc(C45CC6CC7C8CC(CC74)CC5C8C6)cc3)cc(-c3nc(-c4ccccc4)nc(-c4ccccc4)n3)c2)c1]

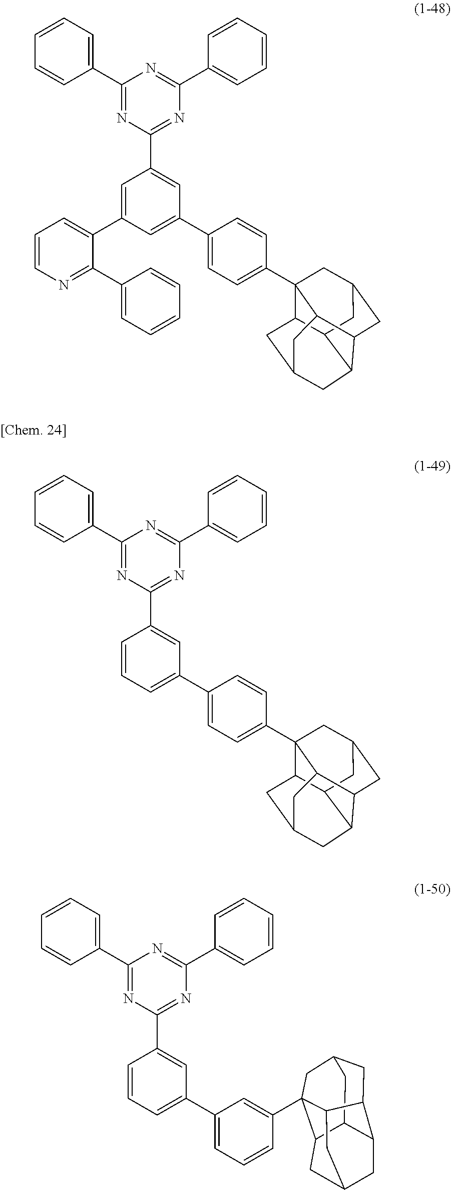 [SMILES: c1ccc(-c2nc(-c3ccccc3)nc(-c3cc(-c4ccc(C56CC7CC8C9CC(CC85)CC6C9C7)cc4)cc(-c4cccnc4-c4ccccc4)c3)n2)cc1 ; c1ccc(-c2nc(-c3ccccc3)nc(-c3cccc(-c4ccc(C56CC7CC8C9CC(CC85)CC6C9C7)cc4)c3)n2)cc1 ; c1ccc(-c2nc(-c3ccccc3)nc(-c3cccc(-c4cccc(C56CC7CC8C9CC(CC85)CC6C9C7)c4)c3)n2)cc1]

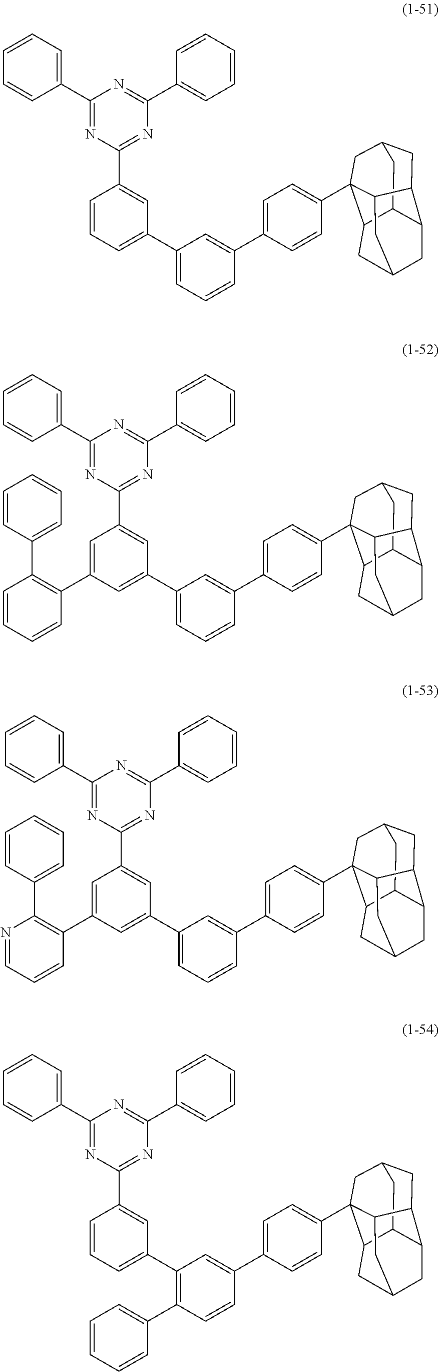 [SMILES: c1ccc(-c2nc(-c3ccccc3)nc(-c3cccc(-c4cccc(-c5ccc(C67CC8CC9C%10CC(CC96)CC7C%10C8)cc5)c4)c3)n2)cc1 ; c1ccc(-c2nc(-c3ccccc3)nc(-c3cc(-c4cccc(-c5ccc(C67CC8CC9C%10CC(CC96)CC7C%10C8)cc5)c4)cc(-c4ccccc4-c4ccccc4)c3)n2)cc1 ; c1ccc(-c2nc(-c3ccccc3)nc(-c3cc(-c4cccc(-c5ccc(C67CC8CC9C%10CC(CC96)CC7C%10C8)cc5)c4)cc(-c4cccnc4-c4ccccc4)c3)n2)cc1 ; c1ccc(-c2nc(-c3ccccc3)nc(-c3cccc(-c4cc(-c5ccc(C67CC8CC9C%10CC(CC96)CC7C%10C8)cc5)ccc4-c4ccccc4)c3)n2)cc1]

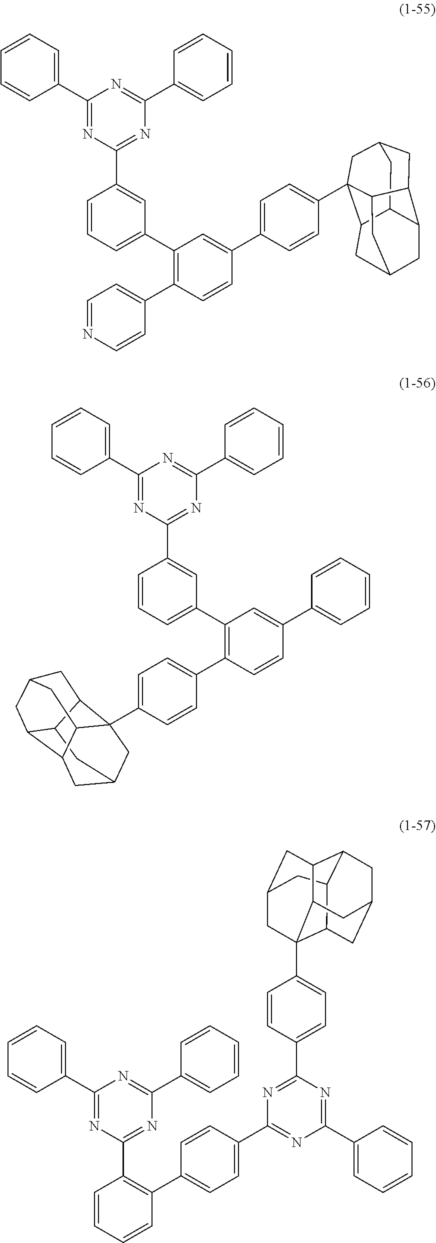 [SMILES: c1ccc(-c2nc(-c3ccccc3)nc(-c3cccc(-c4cc(-c5ccc(C67CC8CC9C%10CC(CC96)CC7C%10C8)cc5)ccc4-c4ccncc4)c3)n2)cc1 ; c1ccc(-c2ccc(-c3ccc(C45CC6CC7C8CC(CC74)CC5C8C6)cc3)c(-c3cccc(-c4nc(-c5ccccc5)nc(-c5ccccc5)n4)c3)c2)cc1 ; c1ccc(-c2nc(-c3ccc(-c4ccccc4-c4nc(-c5ccccc5)nc(-c5ccccc5)n4)cc3)nc(-c3ccc(C45CC6CC7C8CC(CC74)CC5C8C6)cc3)n2)cc1]

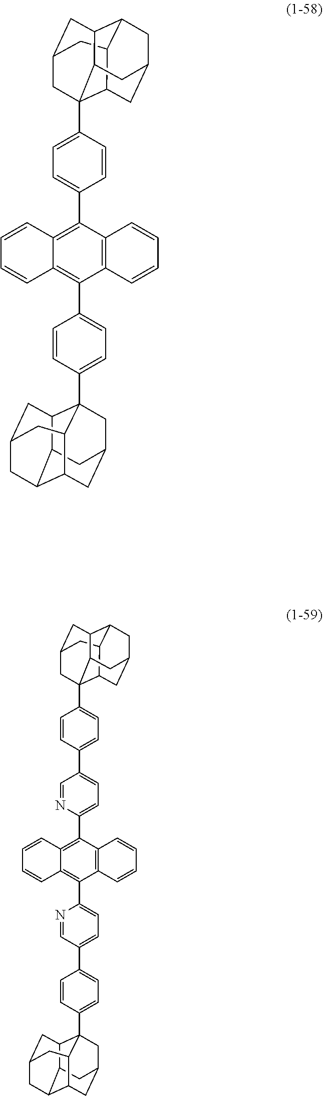

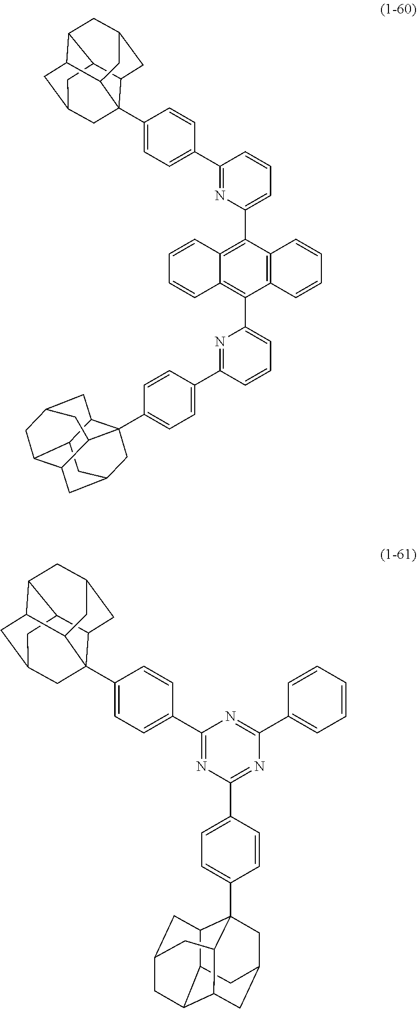 [SMILES: c1cc(-c2ccc(C34CC5CC6C7CC(CC63)CC4C7C5)cc2)nc(-c2c3ccccc3c(-c3cccc(-c4ccc(C56CC7CC8C9CC(CC85)CC6C9C7)cc4)n3)c3ccccc23)c1 ; c1ccc(-c2nc(-c3ccc(C45CC6CC7C8CC(CC74)CC5C8C6)cc3)nc(-c3ccc(C45CC6CC7C8CC(CC74)CC5C8C6)cc3)n2)cc1]

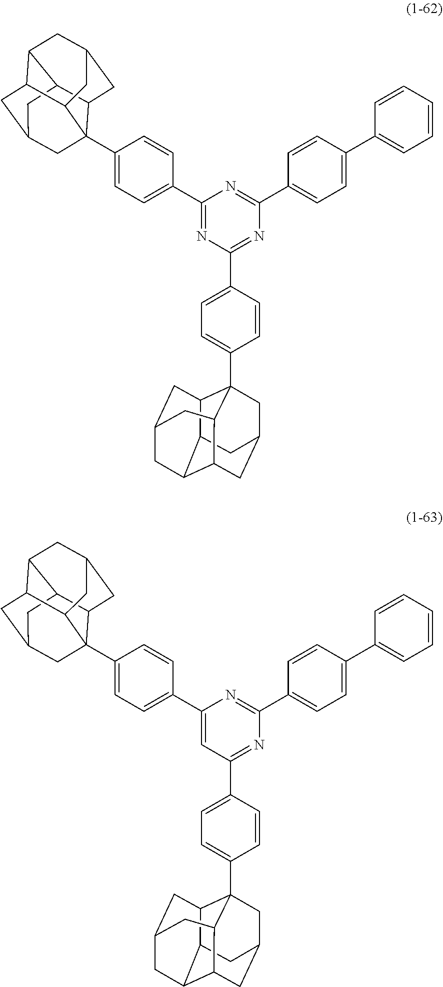 [SMILES: c1ccc(-c2ccc(-c3nc(-c4ccc(C56CC7CC8C9CC(CC85)CC6C9C7)cc4)nc(-c4ccc(C56CC7CC8C9CC(CC85)CC6C9C7)cc4)n3)cc2)cc1 ; c1ccc(-c2ccc(-c3nc(-c4ccc(C56CC7CC8C9CC(CC85)CC6C9C7)cc4)cc(-c4ccc(C56CC7CC8C9CC(CC85)CC6C9C7)cc4)n3)cc2)cc1]

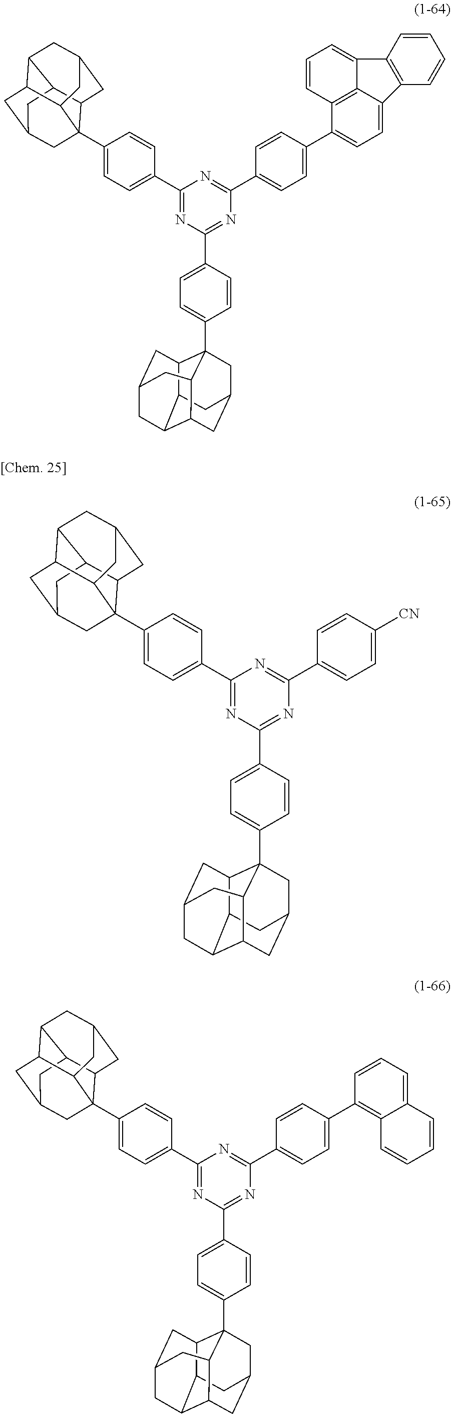 [SMILES: c1ccc2c(c1)-c1cccc3c(-c4ccc(-c5nc(-c6ccc(C78CC9CC%10C%11CC(CC%107)CC8C%11C9)cc6)nc(-c6ccc(C78CC9CC%10C%11CC(CC%107)CC8C%11C9)cc6)n5)cc4)ccc-2c13 ; N#Cc1ccc(-c2nc(-c3ccc(C45CC6CC7C8CC(CC74)CC5C8C6)cc3)nc(-c3ccc(C45CC6CC7C8CC(CC74)CC5C8C6)cc3)n2)cc1 ; c1ccc2c(-c3ccc(-c4nc(-c5ccc(C67CC8CC9C%10CC(CC96)CC7C%10C8)cc5)nc(-c5ccc(C67CC8CC9C%10CC(CC96)CC7C%10C8)cc5)n4)cc3)cccc2c1]

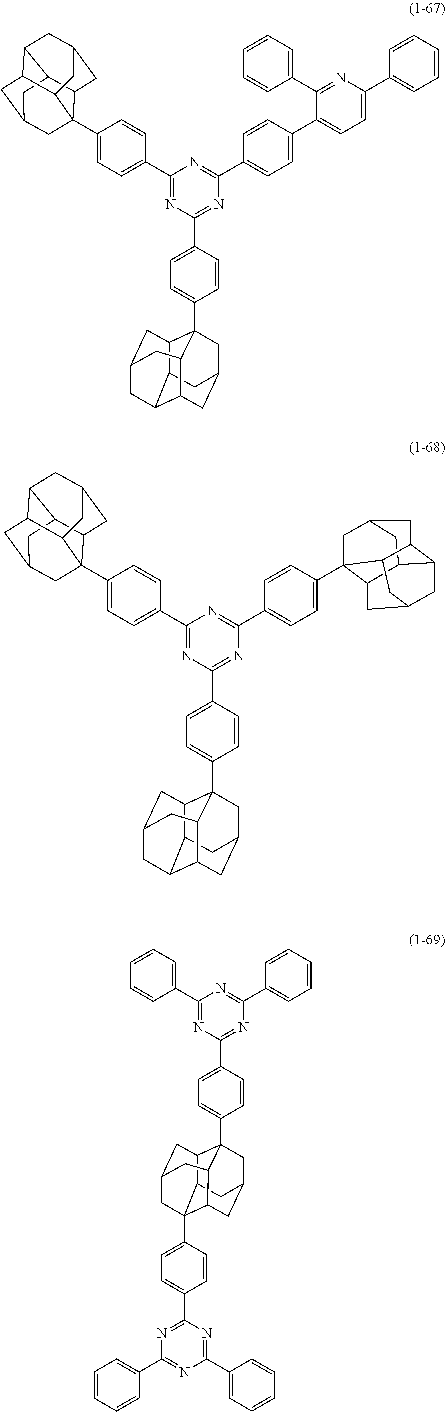 [SMILES: c1ccc(-c2ccc(-c3ccc(-c4nc(-c5ccc(C67CC8CC9C%10CC(CC96)CC7C%10C8)cc5)nc(-c5ccc(C67CC8CC9C%10CC(CC96)CC7C%10C8)cc5)n4)cc3)c(-c3ccccc3)n2)cc1 ; c1cc(C23CC4CC5C6CC(CC52)CC3C6C4)ccc1-c1nc(-c2ccc(C34CC5CC6C7CC(CC63)CC4C7C5)cc2)nc(-c2ccc(C34CC5CC6C7CC(CC63)CC4C7C5)cc2)n1 ; c1ccc(-c2nc(-c3ccccc3)nc(-c3ccc(C45CC6CC7C4CC4CC5C(C6)C7(c5ccc(-c6nc(-c7ccccc7)nc(-c7ccccc7)n6)cc5)C4)cc3)n2)cc1]

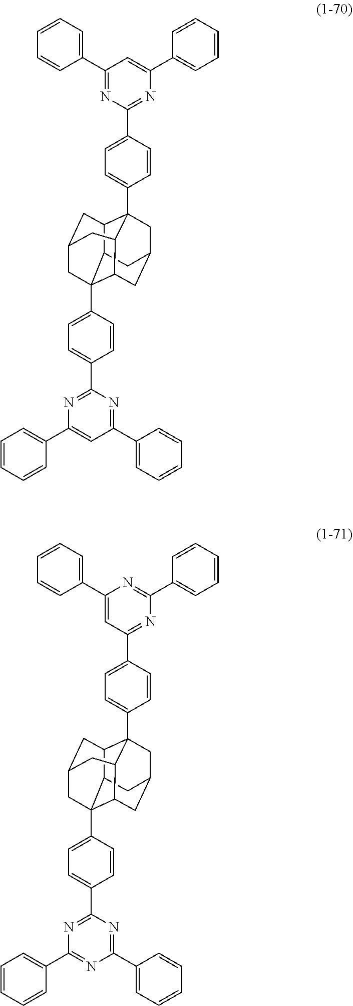 [SMILES: c1ccc(-c2cc(-c3ccccc3)nc(-c3ccc(C45CC6CC7C4CC4CC5C(C6)C7(c5ccc(-c6nc(-c7ccccc7)cc(-c7ccccc7)n6)cc5)C4)cc3)n2)cc1 ; c1ccc(-c2cc(-c3ccc(C45CC6CC7C4CC4CC5C(C6)C7(c5ccc(-c6nc(-c7ccccc7)nc(-c7ccccc7)n6)cc5)C4)cc3)nc(-c3ccccc3)n2)cc1]

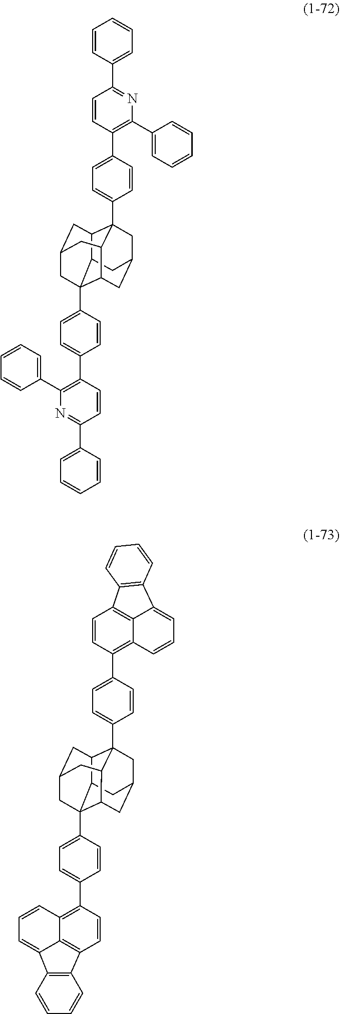

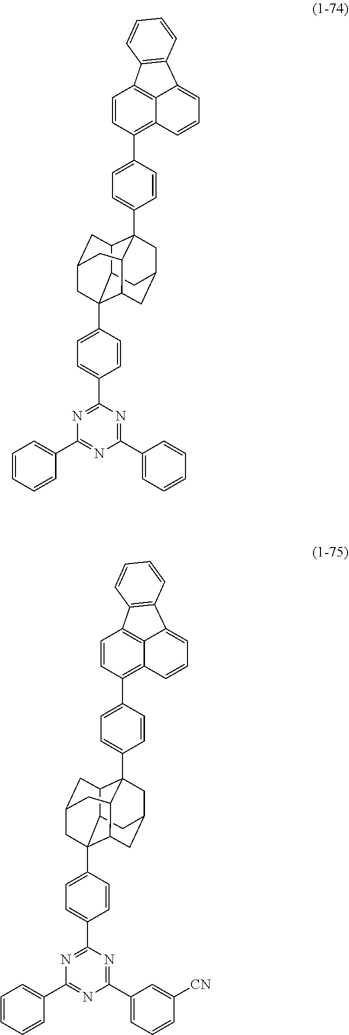 [SMILES: c1ccc(-c2nc(-c3ccccc3)nc(-c3ccc(C45CC6CC7C4CC4CC5C(C6)C7(c5ccc(-c6ccc7c8c(cccc68)-c6ccccc6-7)cc5)C4)cc3)n2)cc1 ; N#Cc1cccc(-c2nc(-c3ccccc3)nc(-c3ccc(C45CC6CC7C4CC4CC5C(C6)C7(c5ccc(-c6ccc7c8c(cccc68)-c6ccccc6-7)cc5)C4)cc3)n2)c1]

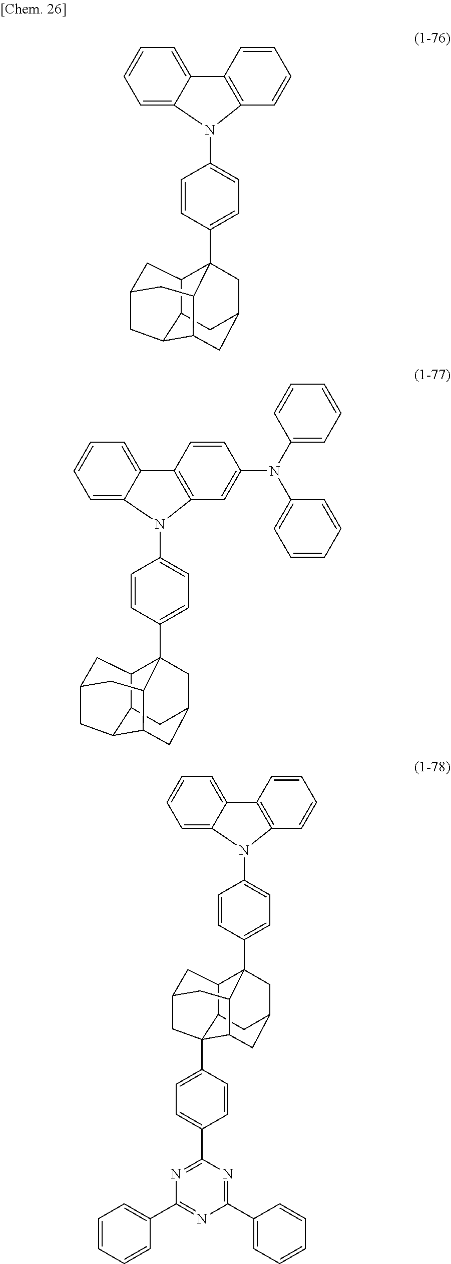

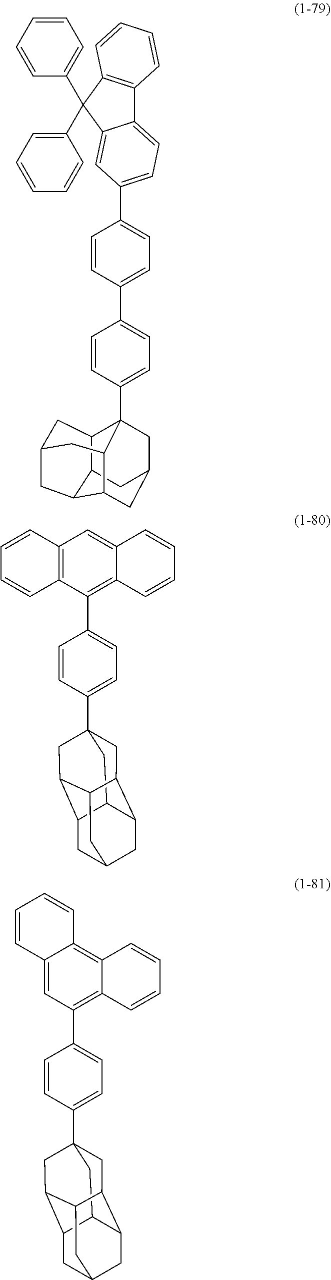

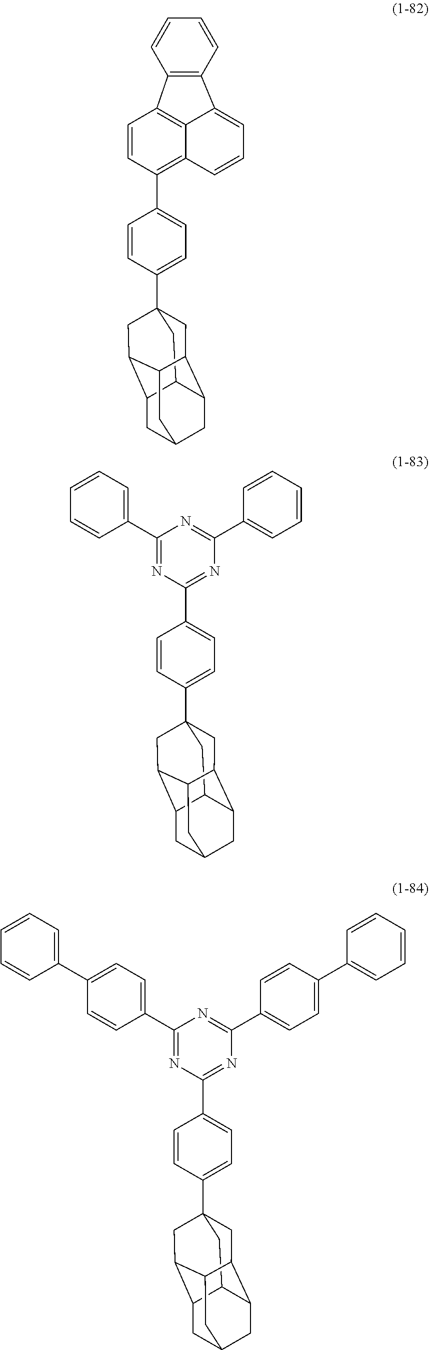

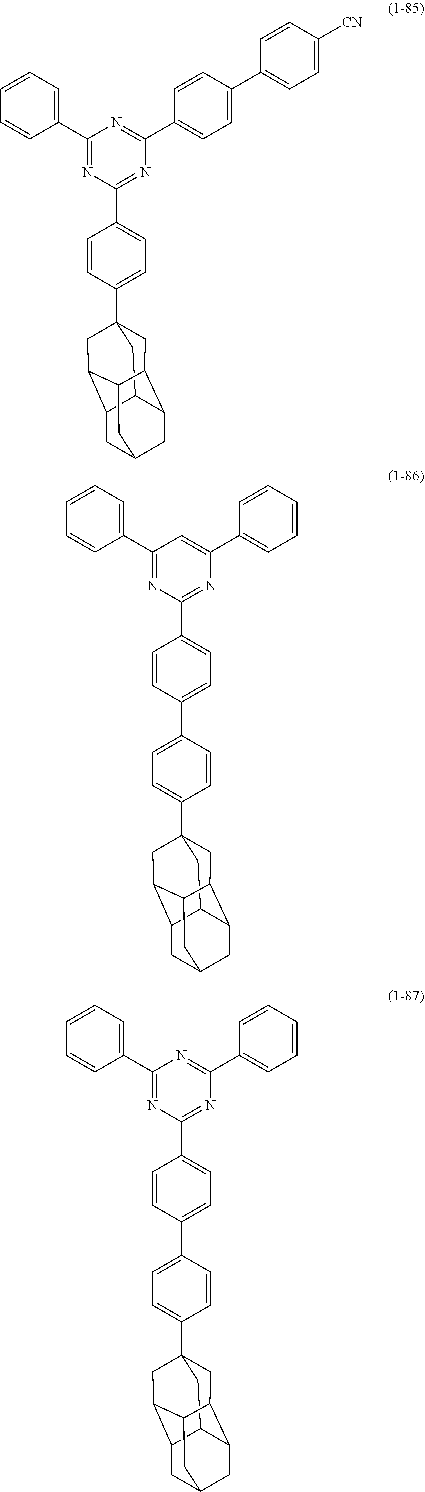

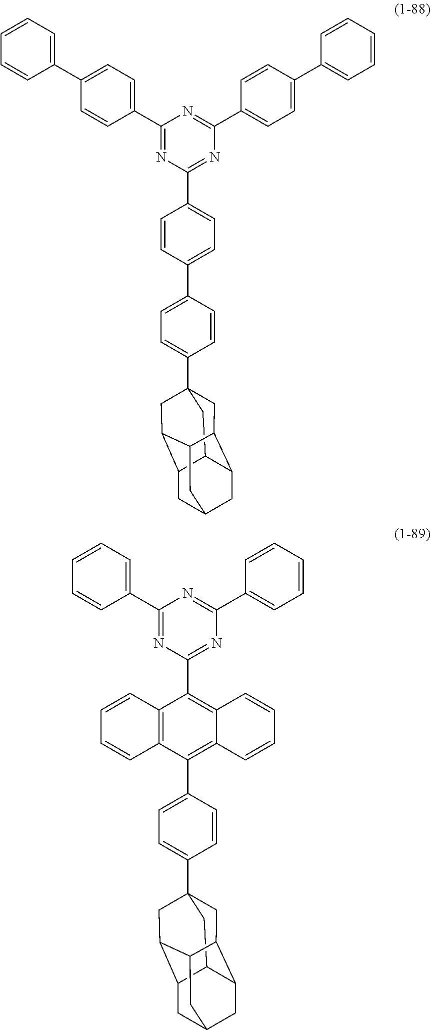 [SMILES: c1ccc(-c2ccc(-c3nc(-c4ccc(-c5ccccc5)cc4)nc(-c4ccc(-c5ccc(C67CC8C9CC%10CC8C(C6)C(C%10)C9C7)cc5)cc4)n3)cc2)cc1 ; c1ccc(-c2nc(-c3ccccc3)nc(-c3c4ccccc4c(-c4ccc(C56CC7C8CC9CC7C(C5)C(C9)C8C6)cc4)c4ccccc34)n2)cc1]

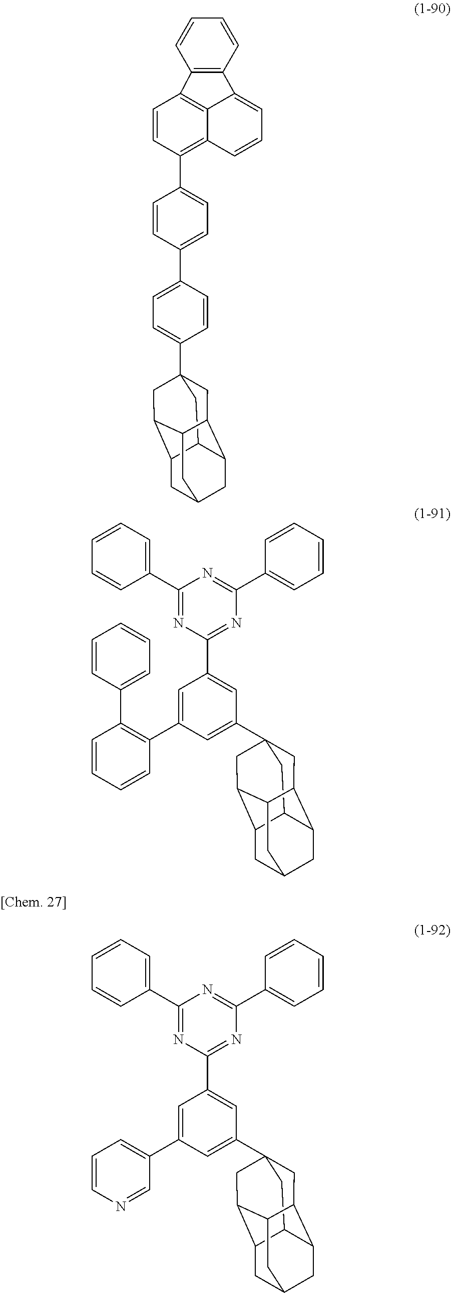

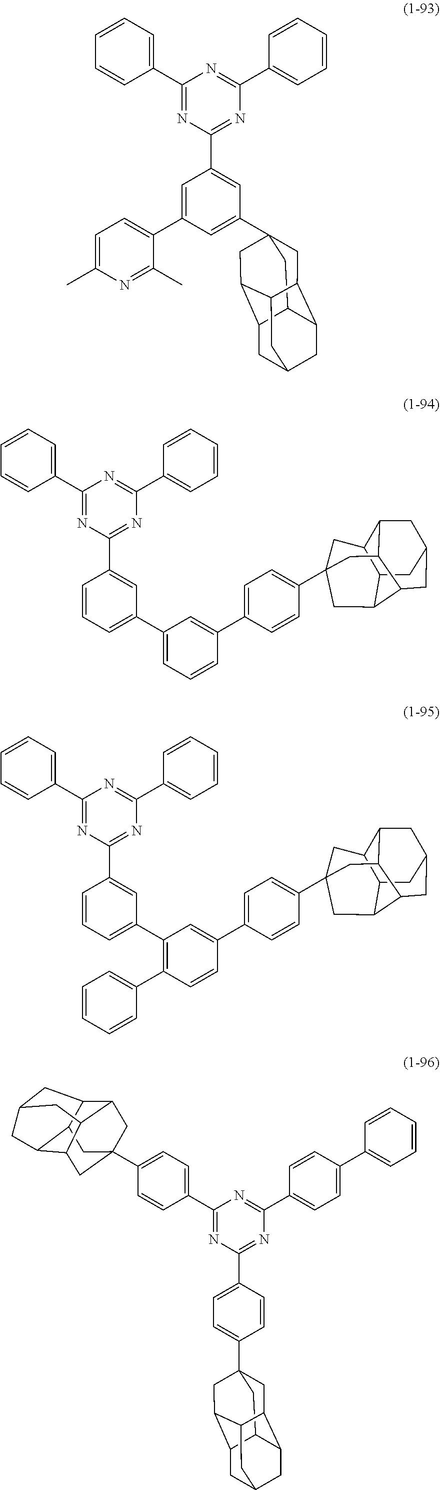 [SMILES: Cc1ccc(-c2cc(-c3nc(-c4ccccc4)nc(-c4ccccc4)n3)cc(C34CC5C6CC7CC5C(C3)C(C7)C6C4)c2)c(C)n1 ; c1ccc(-c2nc(-c3ccccc3)nc(-c3cccc(-c4cccc(-c5ccc(C67CC8C9CC%10CC8C(C6)C(C%10)C9C7)cc5)c4)c3)n2)cc1 ; c1ccc(-c2nc(-c3ccccc3)nc(-c3cccc(-c4cc(-c5ccc(C67CC8C9CC%10CC8C(C6)C(C%10)C9C7)cc5)ccc4-c4ccccc4)c3)n2)cc1 ; c1ccc(-c2ccc(-c3nc(-c4ccc(C56CC7C8CC9CC7C(C5)C(C9)C8C6)cc4)nc(-c4ccc(C56CC7C8CC9CC7C(C5)C(C9)C8C6)cc4)n3)cc2)cc1]

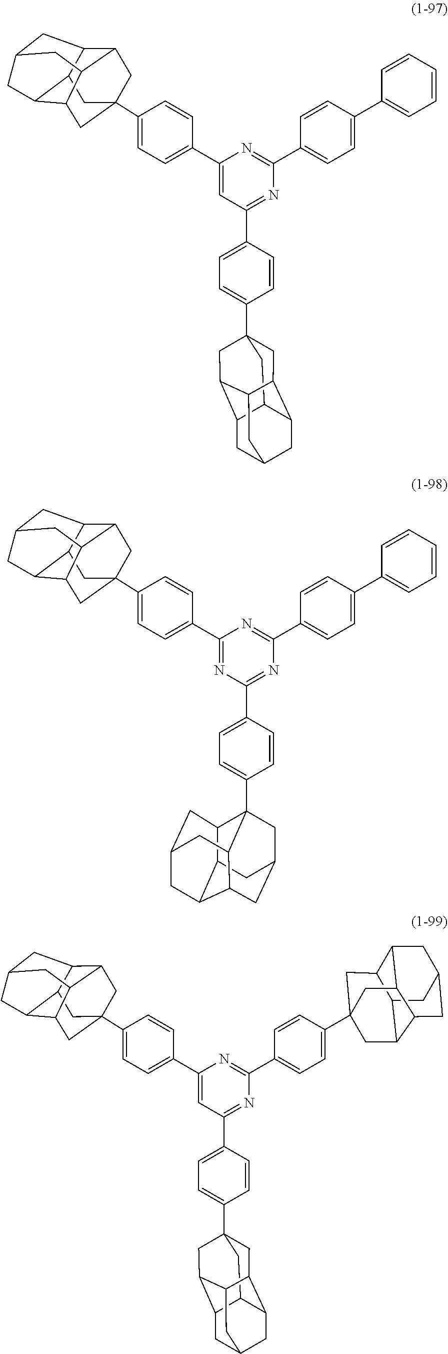 [SMILES: c1ccc(-c2ccc(-c3nc(-c4ccc(C56CC7C8CC9CC7C(C5)C(C9)C8C6)cc4)cc(-c4ccc(C56CC7C8CC9CC7C(C5)C(C9)C8C6)cc4)n3)cc2)cc1 ; CC12CC3CC4C1CC1CC4C(c4ccc(-c5nc(-c6ccc(-c7ccccc7)cc6)nc(-c6ccc(C78CC9C%10CC%11CC9C(C7)C(C%11)C%10C8)cc6)n5)cc4)(C3)C2C1 ; CC12CC3CC4C5CC(c6ccc(-c7nc(-c8ccc(C9%10CC%11C%12CC%13CC%11C(C9)C(C%13)C%12C%10)cc8)cc(-c8ccc(C9%10CC%11C%12CC%13CC%11C(C9)C(C%13)C%12C%10)cc8)n7)cc6)(CC41)CC2C5C3]

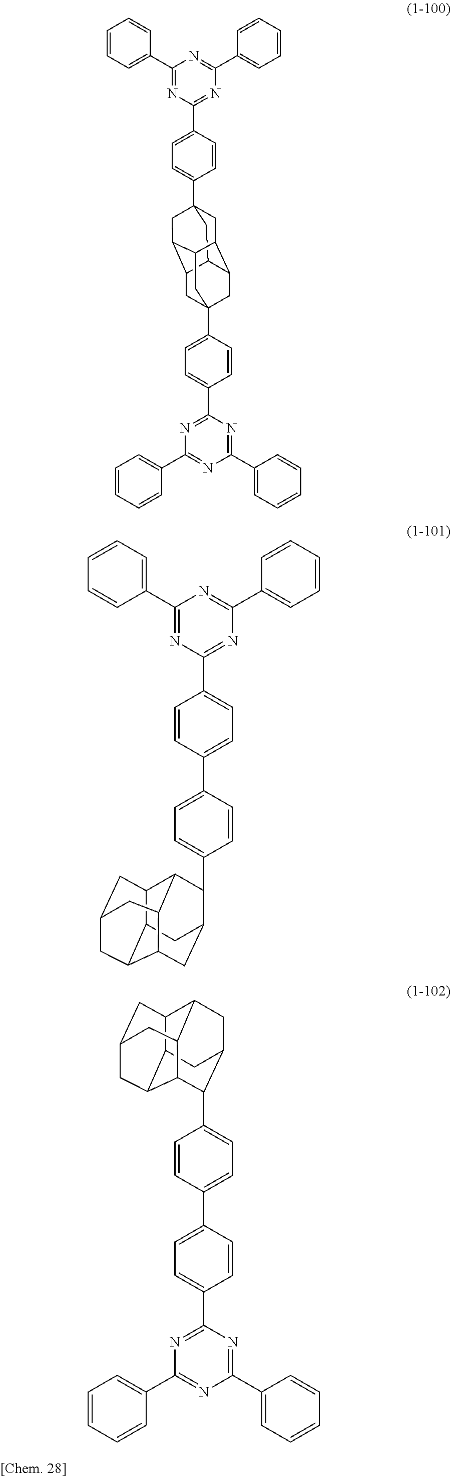

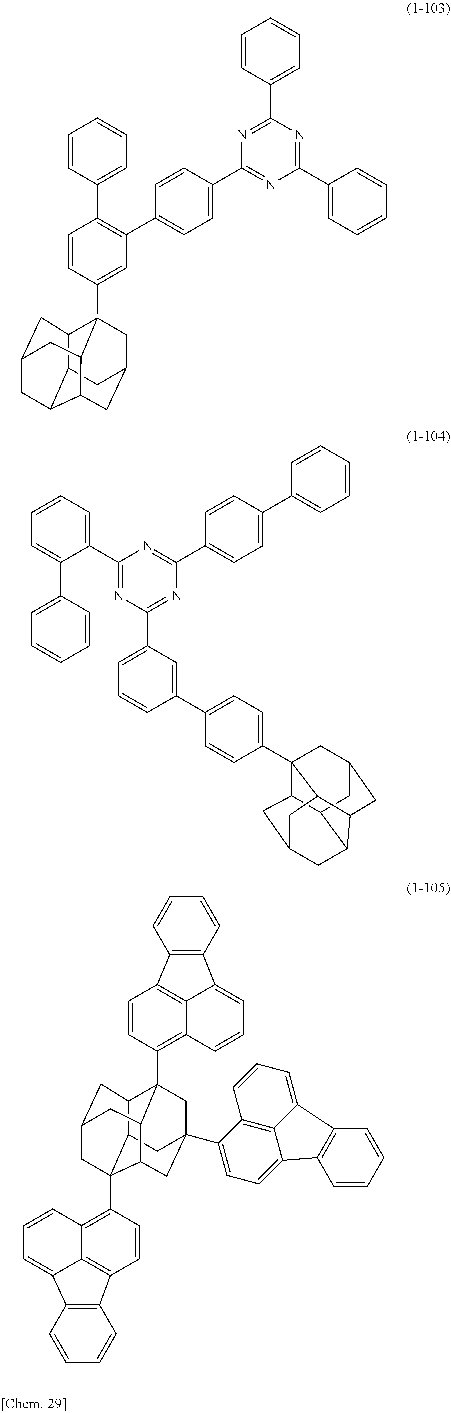

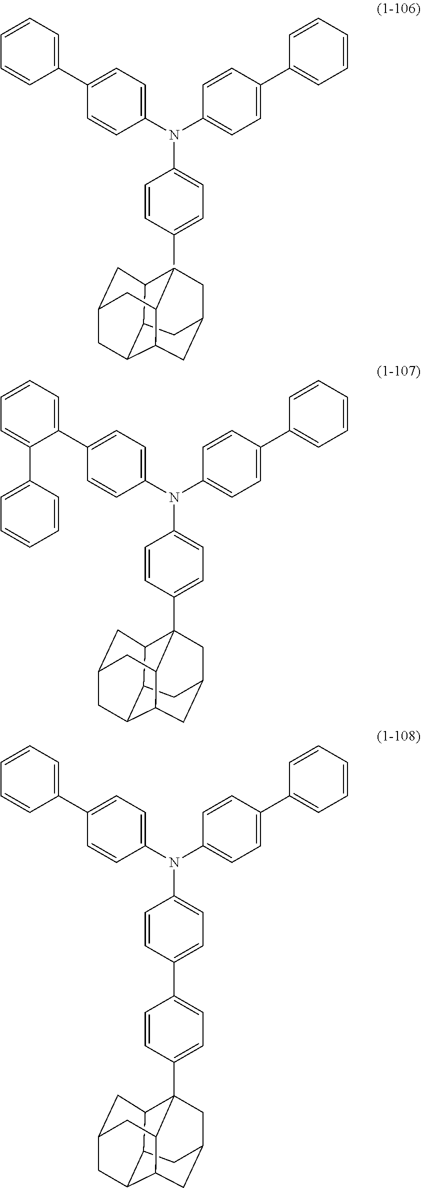 [SMILES: CC12CC3CC4C5CC(CC41)CC2(c1ccc(N(c2ccc(-c4ccccc4)cc2)c2ccc(-c4ccccc4)cc2)cc1)C5C3 ; CC12CC3CC4C5CC(CC41)CC2(c1ccc(N(c2ccc(-c4ccccc4)cc2)c2ccc(-c4ccccc4-c4ccccc4)cc2)cc1)C5C3 ; CC12CC3CC4C5CC(CC41)CC2(c1ccc(-c2ccc(N(c4ccc(-c6ccccc6)cc4)c4ccc(-c6ccccc6)cc4)cc2)cc1)C5C3]

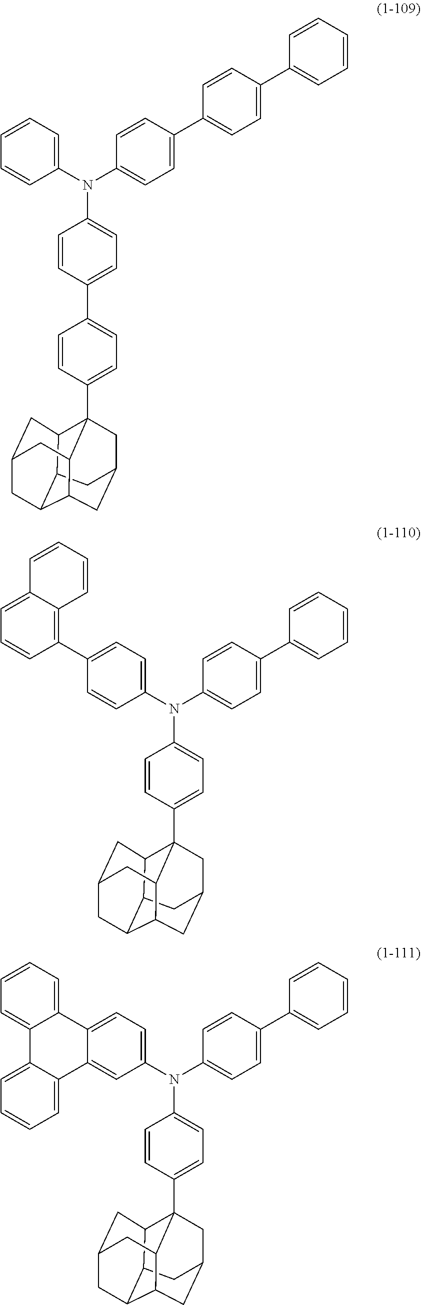

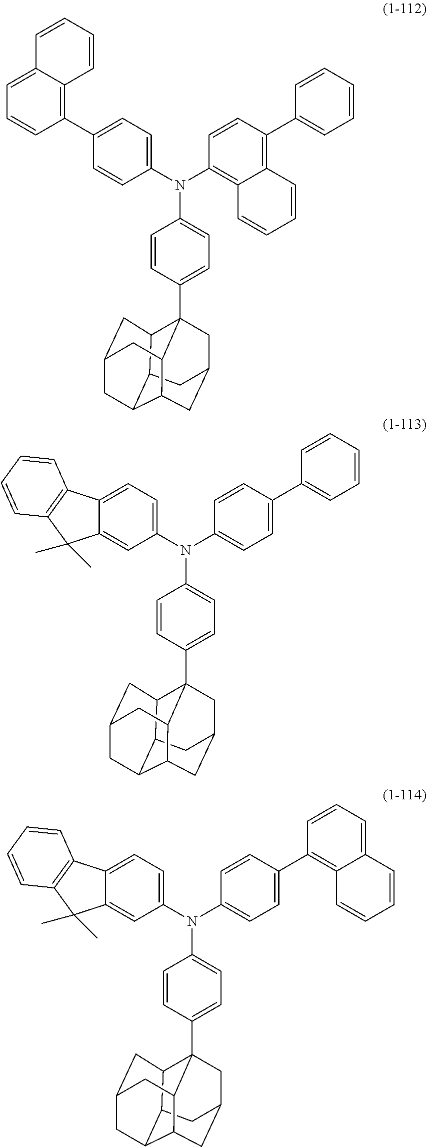 [SMILES: CC12CC3CC4C5CC(CC41)CC2(c1ccc(N(c2ccc(-c4cccc6ccccc46)cc2)c2ccc(-c4ccccc4)c4ccccc24)cc1)C5C3 ; CC1(C)c2ccccc2-c2ccc(N(c3ccc(-c4ccccc4)cc3)c3ccc(C45CC6CC7C8CC(CC74)CC5(C)C8C6)cc3)cc21 ; CC1(C)c2ccccc2-c2ccc(N(c3ccc(-c4cccc5ccccc45)cc3)c3ccc(C45CC6CC7C8CC(CC74)CC5(C)C8C6)cc3)cc21]

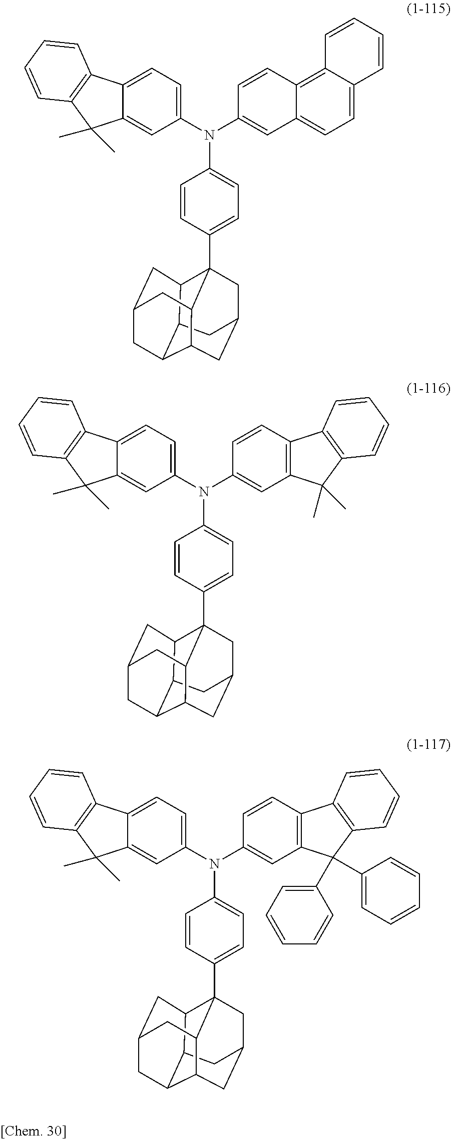 [SMILES: CC1(C)c2ccccc2-c2ccc(N(c3ccc(C45CC6CC7C8CC(CC74)CC5(C)C8C6)cc3)c3ccc4c(ccc5ccccc54)c3)cc21 ; CC1(C)c2ccccc2-c2ccc(N(c3ccc(C45CC6CC7C8CC(CC74)CC5(C)C8C6)cc3)c3ccc4c(c3)C(C)(C)c3ccccc3-4)cc21 ; CC1(C)c2ccccc2-c2ccc(N(c3ccc(C45CC6CC7C8CC(CC74)CC5(C)C8C6)cc3)c3ccc4c(c3)C(c3ccccc3)(c3ccccc3)c3ccccc3-4)cc21]

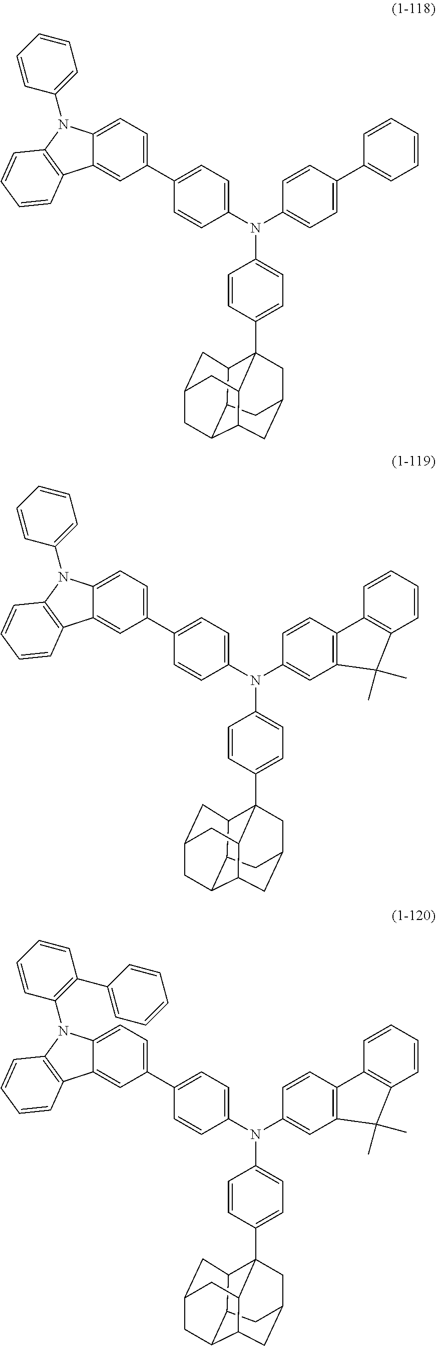 [SMILES: CC12CC3CC4C5CC(CC41)CC2(c1ccc(N(c2ccc(-c4ccccc4)cc2)c2ccc(-c4ccc6c(c4)c4ccccc4n6-c4ccccc4)cc2)cc1)C5C3 ; CC1(C)c2ccccc2-c2ccc(N(c3ccc(-c4ccc5c(c4)c4ccccc4n5-c4ccccc4)cc3)c3ccc(C45CC6CC7C8CC(CC74)CC5(C)C8C6)cc3)cc21 ; CC1(C)c2ccccc2-c2ccc(N(c3ccc(-c4ccc5c(c4)c4ccccc4n5-c4ccccc4-c4ccccc4)cc3)c3ccc(C45CC6CC7C8CC(CC74)CC5(C)C8C6)cc3)cc21]

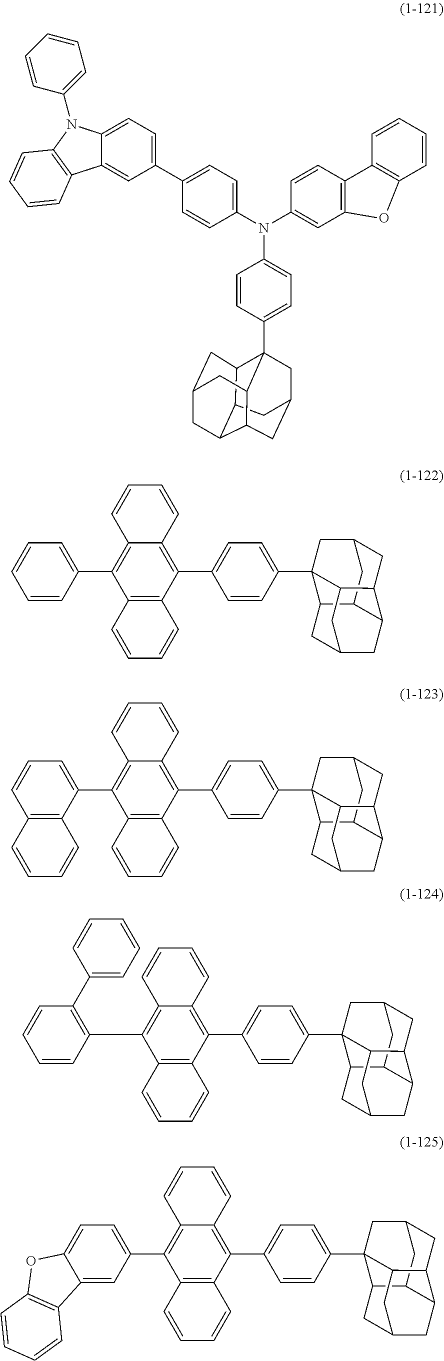

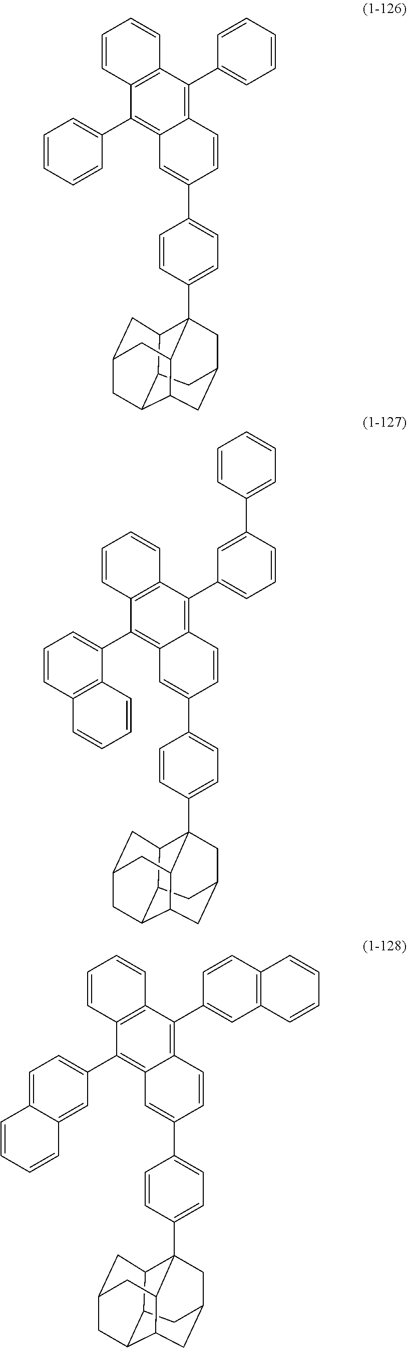

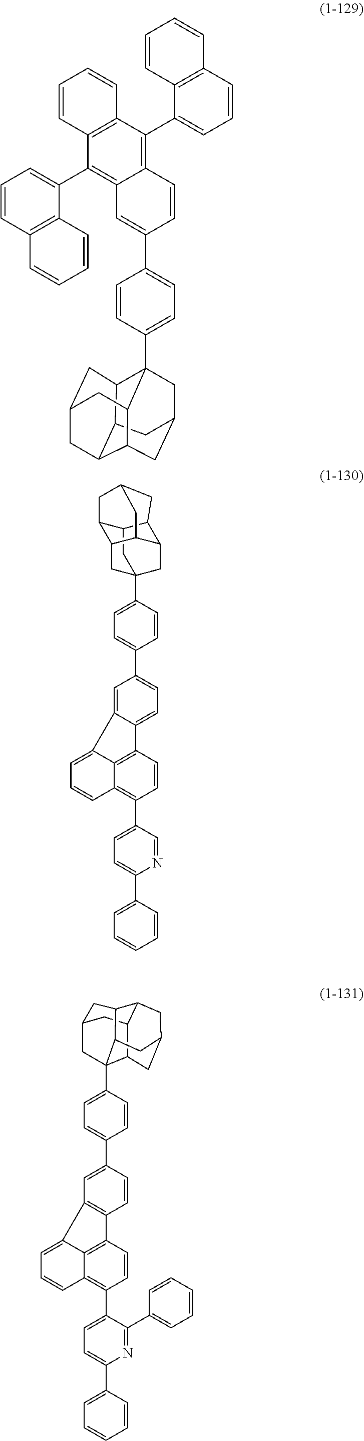

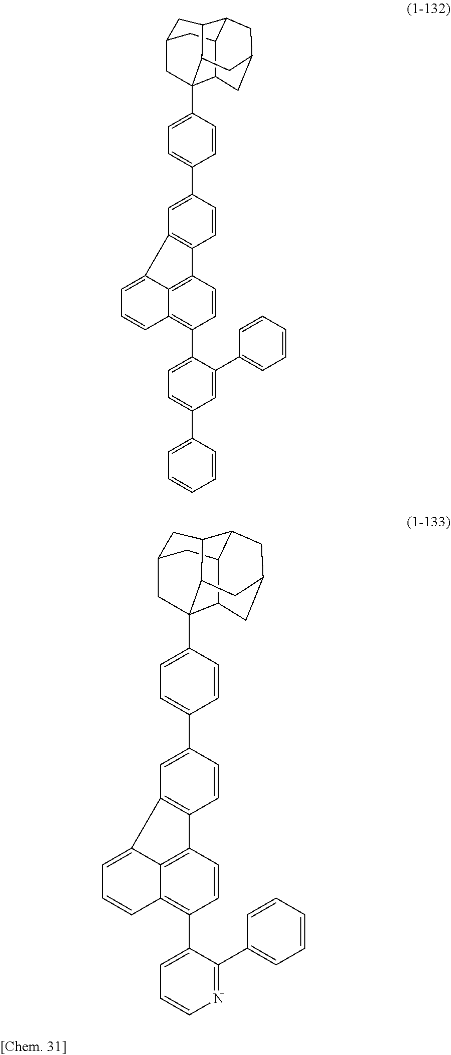

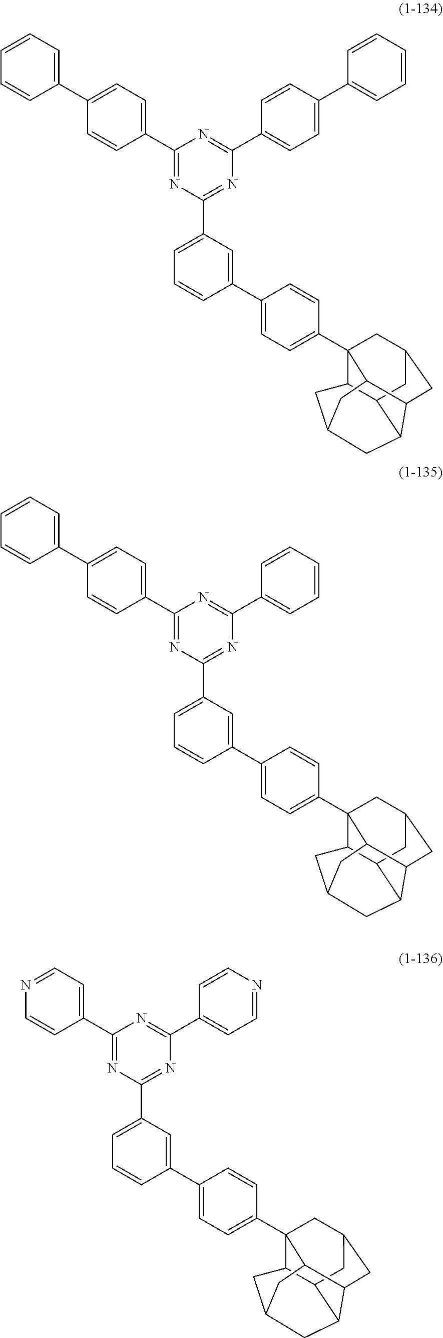 [SMILES: c1ccc(-c2ccc(-c3nc(-c4ccc(-c5ccccc5)cc4)nc(-c4cccc(-c5ccc(C67CC8CC9C%10CC(CC96)CC7C%10C8)cc5)c4)n3)cc2)cc1 ; c1ccc(-c2ccc(-c3nc(-c4ccccc4)nc(-c4cccc(-c5ccc(C67CC8CC9C%10CC(CC96)CC7C%10C8)cc5)c4)n3)cc2)cc1 ; c1cc(-c2ccc(C34CC5CC6C7CC(CC63)CC4C7C5)cc2)cc(-c2nc(-c3ccncc3)nc(-c3ccncc3)n2)c1]

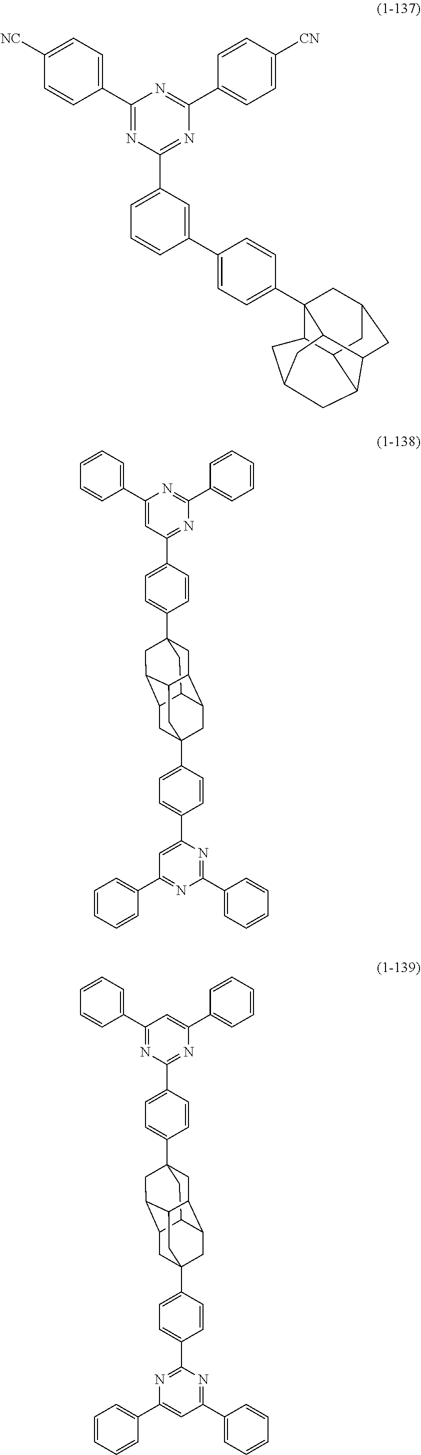 [SMILES: N#Cc1ccc(-c2nc(-c3ccc(C#N)cc3)nc(-c3cccc(-c4ccc(C56CC7CC8C9CC(CC85)CC6C9C7)cc4)c3)n2)cc1 ; c1ccc(-c2cc(-c3ccc(C45CC6C7CC8(c9ccc(-c%10cc(-c%11ccccc%11)nc(-c%11ccccc%11)n%10)cc9)CC6C(C4)C(C8)C7C5)cc3)nc(-c3ccccc3)n2)cc1 ; c1ccc(-c2cc(-c3ccccc3)nc(-c3ccc(C45CC6C7CC8(c9ccc(-c%10nc(-c%11ccccc%11)cc(-c%11ccccc%11)n%10)cc9)CC6C(C4)C(C8)C7C5)cc3)n2)cc1]

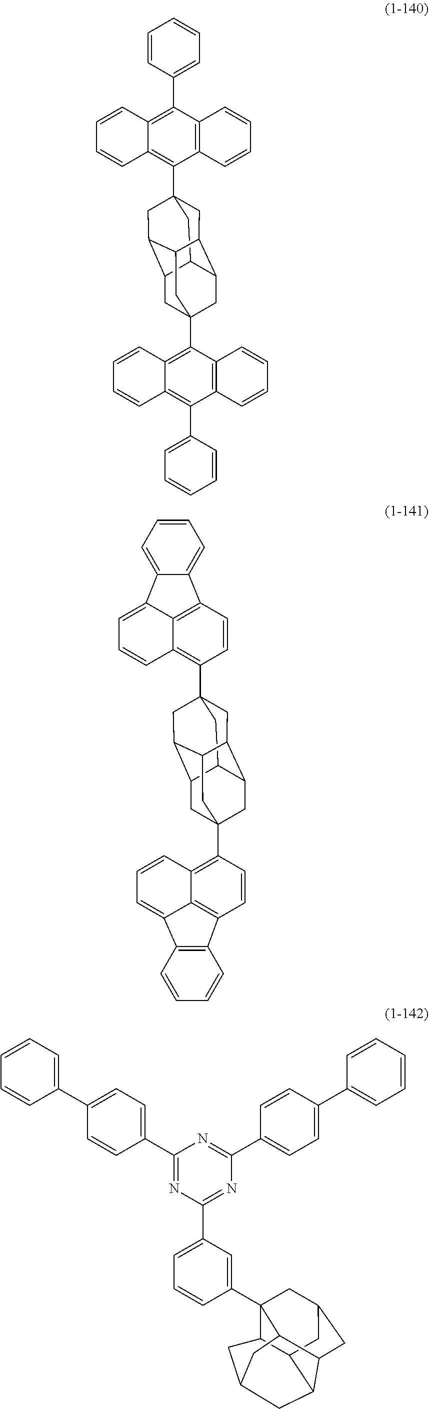

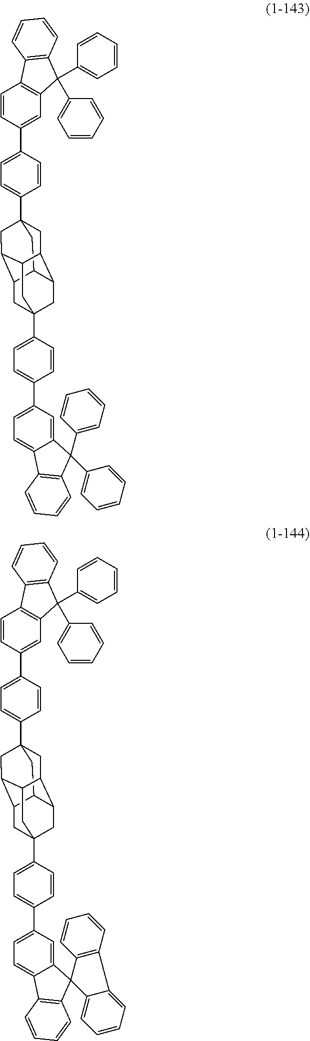

### [Method for Producing Diamantane Compound]

A method for producing the diamantane compound will be described.

The diamantane compound according to the present invention can be produced by an appropriate combination of known reactions (e.g., the Suzuki-Miyaura cross-coupling reaction) generally used by those skilled in the art as methods for synthesizing aromatic compounds.

For example, the diamantane compound can be produced by methods presented as synthetic pathways (i) to (iii) below.

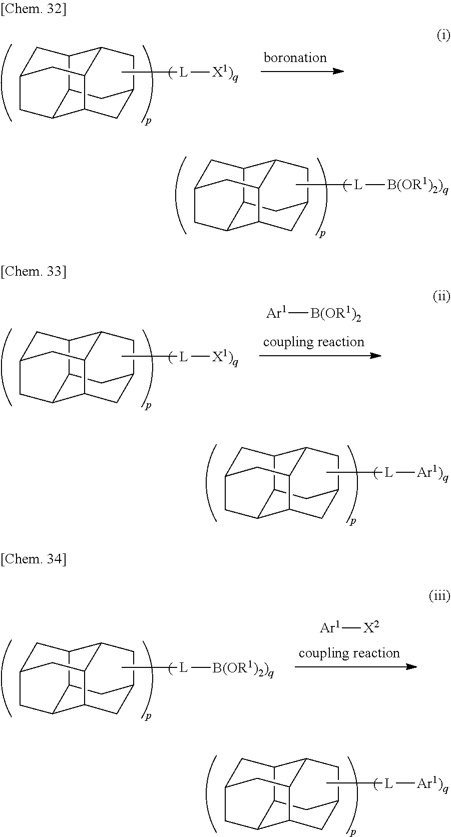

In the synthetic pathways (i) to (iii) above, L, Ar¹, R¹, X¹, X², p and q are as follows.

Each L independently represents a single bond or
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

Each Ar¹ independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

X¹ and X² each represent a halogen atom or a trifluoromethanesulfonyloxy group.

R¹ represents a hydrogen atom, an alkyl group having 1 to 4 carbon atoms or a phenyl group. Two R¹'s of B(OR¹)₂ may be the same or different. The two R¹'s may be combined to form a ring including the oxygen atom and the boron atom.

p and q each represent an integer of 1 to 3. When p is 1, q represents an integer of 1, 2 or 3, and when q is 1, p represents an integer of 1, 2 or 3.

-L-Ar¹ has the same meaning as —Ar in formula (2).

The halogen atom represented by X¹ and X² is, for example, a fluorine atom, a chlorine atom, a bromine atom or an iodine atom, and is preferably a chlorine atom or a bromine atom from the viewpoint of its low raw material cost.

Examples of B(OR¹)₂ in the boron compound include, but are not limited to, B(OH)₂, B(OMe)₂, B(OiPr)₂, B(OBu)₂ and B(OPh)₂. Me represents a methyl group, iPr represents an isopropyl group, Bu represents a butyl group, and Ph represents a phenyl group. Examples of B(OR¹)₂ in the case where the two R¹'s are combined to form a ring including the oxygen atom and the boron atom include, but are not limited to, the following groups represented by (I) to (VI), and the group represented by (II) is preferred for a good yield.

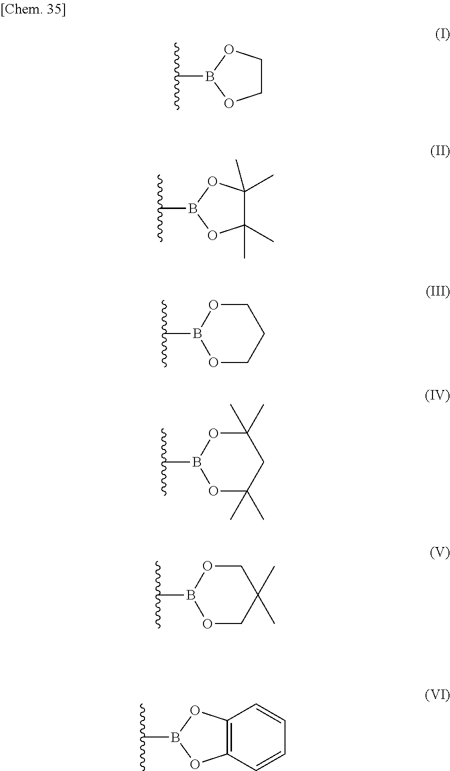

The coupling reactions in the synthetic pathways (ii) and (iii) are each a reaction of an aryl halide compound and a boron compound in the presence of a palladium catalyst and a base, and the prevalent reaction conditions for the Suzuki-Miyaura reaction can be applied thereto.

The boron compound used in the coupling reactions can be produced, for example, in accordance with a method disclosed in The Journal of Organic Chemistry, vol. 60, p. 7508, 1995 or The Journal of Organic Chemistry, vol. 65, p. 164, 2000. Alternatively, a commercially available product may be used.

The aryl halide used in the coupling reactions can be produced, for example, in accordance with Journal of the American Chemical Society, vol. 74, p. 6289, 1952 or Synlett, p. 808, 2002. Alternatively, a commercially available product may be used. The molar equivalent of the aryl halide for use is not particularly limited, but for a good reaction yield, the aryl halide is preferably used in an amount of 0.5 to 3.0 molar equivalents per molar equivalent of the boron compound.

Specific examples of the palladium catalyst used in the coupling reactions described above include, but are not limited to, palladium salts such as palladium chloride, palladium acetate, palladium trifluoroacetate and palladium nitrate. Further examples include complex compounds such as n-allylpalladium chloride dimer, palladium acetylacetonate, tris(dibenzylideneacetone) dipalladium, bis(dibenzylideneacetone) palladium, dichlorobis(acetonitrile) palladium and dichlorobis(benzonitrile) palladium, and palladium complexes having a tertiary phosphine as a ligand, such as dichlorobis(triphenylphosphine) palladium, tetrakis(triphenylphosphine) palladium, dichloro(1,1′-bis(diphenylphosphino)ferrocene) palladium, bis(tri-tert-butylphosphine) palladium, bis(tricyclohexylphosphine) palladium and dichlorobis(tricyclohexylphosphine) palladium, which can be prepared in a reaction system by adding a tertiary phosphine to a palladium salt or a complex compound. Examples of tertiary phosphines that can be used here include triphenylphosphine, trimethylphosphine, tributylphosphine, tri(tert-butyl)phosphine, tricyclohexylphosphine, tert-butyldiphenylphosphine, 9,9-dimethyl-4,5-bis(diphenylphosphino)xanthene, 2-(diphenylphosphino)-2′-(N,N-dimethylamino)biphenyl, 2-(di-tert-butylphosphino)biphenyl, 2-(dicyclohexylphosphino)biphenyl, bis(diphenylphosphino)methane, 1,2-bis(diphenylphosphino)ethane, 1,3-bis(diphenylphosphino)propane, 1,4-bis(diphenylphosphino)butane, 1,1′-bis(diphenylphosphino)ferrocene, tri(2-furyl)phosphine, tri(o-tolyl)phosphine, tris(2,5-xylyl)phosphine, (±)-2,2′-bis(diphenylphosphino)-1,1′-binaphthyl and 2-dicyclohexylphosphino-2′,4′,6′-triisopropylbiphenyl.

In particular, palladium complexes having a tertiary phosphine as a ligand are preferred for a good yield, and palladium complexes having 2-dicyclohexylphosphino-2′,4′,6′-triisopropylbiphenyl as a ligand are more preferred. The molar ratio between a tertiary phosphine and a palladium salt or complex compound is preferably in the range of 1:10 to 10:1, more preferably in the range of 1:2 to 3:1 for a good yield. The amount of the palladium catalyst used in the coupling reactions described above is not particularly limited, but for a good yield, the molar equivalent of the palladium catalyst is preferably in the range of 0.005 to 0.5 per molar equivalent of the boron compound.

Examples of the base used in the coupling reactions described above include, but are not limited to, metal hydroxides such as sodium hydroxide, potassium hydroxide and calcium hydroxide, metal carbonates such as sodium carbonate, potassium carbonate, lithium carbonate and cesium carbonate, metal acetates such as potassium acetate and sodium acetate, metal phosphates such as potassium phosphate and sodium phosphate, metal fluorides such as sodium fluoride, potassium fluoride and cesium fluoride, and metal alkoxides such as sodium methoxide, potassium methoxide, sodium ethoxide, potassium isopropyloxide and potassium tert-butoxide. In particular, for a good reaction yield, metal carbonates and metal phosphates are preferred, and potassium carbonate or potassium phosphate is more preferred. The amount of the base used is not particularly limited, but for a good reaction yield, the molar ratio between the base and the boron compound is preferably in the range of 1:2 to 10:1, more preferably in the range of 1:1 to 4:1.

The coupling reactions described above can be carried out in a solvent.

Examples of solvents that can be used in the coupling reactions described above include water, ethers such as diisopropyl ether, dibutyl ether, cyclopentyl methyl ether (CPME), tetrahydrofuran (THF), 2-methyltetrahydrofuran, 1,4-dioxane and dimethoxyethane, aromatic hydrocarbons such as benzene, toluene, xylene, mesitylene and tetralin, carbonates such as ethylene carbonate, propylene carbonate, dimethyl carbonate, diethyl carbonate, ethylmethyl carbonate and 4-fluoroethylene carbonate, esters such as ethyl acetate, butyl acetate, methyl propionate, ethyl propionate, methyl butyrate and γ-lactone, amides such as N,N-dimethylformamide (DMF), dimethylacetamide (DMAc) and N-methylpyrrolidone (NMP), ureas such as N,N,N′,N′-tetramethylurea (TMU) and N,N′-dimethylpropyleneurea (DMPU), dimethylsulfoxide (DMSO), and alcohols such as methanol, ethanol, isopropyl alcohol, butanol, octanol, benzyl alcohol, ethylene glycol, propylene glycol, diethylene glycol, triethylene glycol and 2,2,2-trifluoroethanol, and these may be used as a mixture at any ratio. The amount of solvent used is not particularly limited. Of these, water, ethers, amides, alcohols and solvent mixtures thereof are preferred for a good reaction yield, and a solvent mixture of THF and water is more preferred.

The coupling reactions described above can be carried out at a temperature appropriately selected from 0° C. to 200° C., and for a good reaction yield, the coupling reactions are preferably carried out at a temperature appropriately selected from 100° C. to 160° C.

The coupling reactions described above are achieved by performing a normal treatment after completion of the coupling reactions. If necessary, purification may be performed by, for example, recrystallization, column chromatography, sublimation or preparative HPLC.

### [Intermediate of Diamantane Compound]

The diamantane compound represented by formula (5) or (6) below used in the synthetic pathways (i) to (iii) given above is an intermediate effective in producing the diamantane compound according to the present invention. Using this intermediate can provide the diamantane compound according to the present invention with efficiency and in high yield.

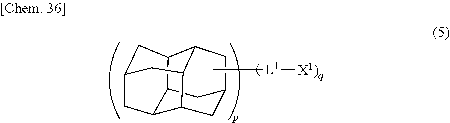

(In a diamantane ring in formula (5), carbon atoms other than a carbon atom bonded to an -L¹-X¹ group are optionally substituted with an aryl group having 6 to 12 carbon atoms.

Each L¹ in formula (5) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

The total number of carbon atoms constituting L¹ is 8 or more.

X¹ in formula (5) represents a halogen atom or a trifluoromethanesulfonyloxy group.

In formula (5), p and q each represent an integer of 1 to 3, when p is 1, q represents an integer of 1, 2 or 3, and when q is 1, p represents an integer of 1, 2 or 3.)

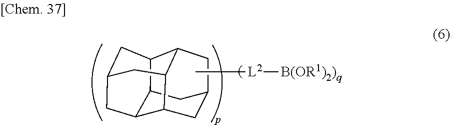

(In a diamantane ring in formula (6), carbon atoms other than a carbon atom bonded to an -L²-B(OR¹)₂ group are optionally substituted with an aryl group having 6 to 12 carbon atoms.

Each L² in formula (6) independently represents a single bond or
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii).

R¹ in formula (6) represents a hydrogen atom, an alkyl group having 1 to 4 carbon atoms or a phenyl group. Two R¹'s of B(OR¹)₂ may be the same or different. The two R¹'s may be combined to form a ring including the oxygen atom and the boron atom.

In formula (6), p and q each represent an integer of 1 to 3, when p is 1, q represents an integer of 1, 2 or 3, and when q is 1, p represents an integer of 1, 2 or 3.)

### <Organic Electroluminescent Device Material>

The diamantane compound is useful as an organic electroluminescent device material. The diamantane compound can be used as, for example, an electron transport material for an organic electroluminescent device or a hole transport material for an organic electroluminescent device. With an organic electroluminescent device material comprising the diamantane compound, an organic electroluminescent device that exhibits high luminous efficiency, more preferably, a low drive voltage and high luminous efficiency, and that can be used in various applications or various environments can be produced.

### <Organic Electroluminescent Device>

Hereinafter, an organic electroluminescent device comprising the diamantane compound (hereinafter also referred to simply as an organic electroluminescent device) will be described.

An organic electroluminescent device according to an aspect of the present invention comprises the diamantane compound.

Examples of configurations of the organic electroluminescent device include, but are not limited to, the following configurations (i) to (v).
(i): Anode/light-emitting layer/cathode
(ii): Anode/hole transport layer/light-emitting layer/cathode
(iii): Anode/light-emitting layer/electron transport layer/cathode
(iv): Anode/hole transport layer/light-emitting layer/electron transport layer/cathode
(v): Anode/hole injection layer/hole transport layer/light-emitting layer/electron transport layer/electron injection layer/cathode

The diamantane compound may be contained in any of the above layers, but for the organic electroluminescent device to have excellent light-emission characteristics, in one embodiment, the diamantane compound is preferably contained in at least one layer selected from the group consisting of a light-emitting layer and layers disposed between the light-emitting layer and a cathode.

In another embodiment, the diamantane compound is preferably contained in at least one layer selected from the group consisting of a light-emitting layer and layers disposed between the light-emitting layer and an anode.

Thus, in the case of the configurations (i) to (v) above, in the one embodiment, the diamantane compound is preferably contained in at least one layer selected from the group consisting of a light-emitting layer, an electron transport layer and an electron injection layer.

In the other embodiment, the diamantane compound is preferably contained in at least one layer selected from the group consisting of a light-emitting layer, a hole transport layer and a hole injection layer.

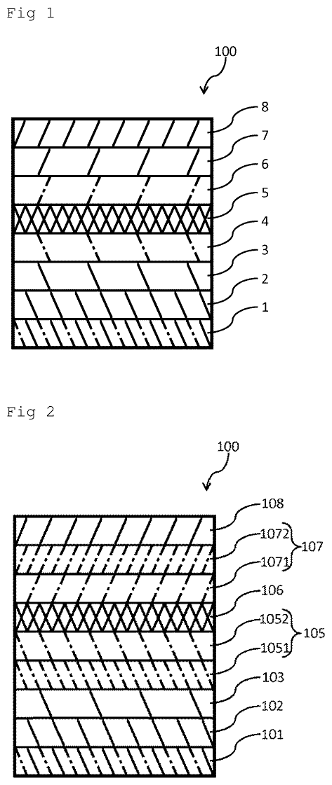

Hereinafter, the organic electroluminescent device according to an aspect of the present invention will be described in more detail with reference to FIG. 1 in the context of the configuration (v).

Although an organic electroluminescent device 100 shown in FIG. 1 has what is called a bottom-emission device structure, the organic electroluminescent device according to an aspect of the present invention need not have a bottom-emission device structure. That is, the organic electroluminescent device according to an aspect of the present invention may have any other known device structure such as a top-emission device structure.

FIG. 1 is a schematic sectional view showing an example of a layered structure of the organic electroluminescent device comprising the diamantane compound according to an aspect of the present invention.

The organic electroluminescent device 100 includes a substrate 1, an anode 2, a hole injection layer 3, a hole transport layer 4, a light-emitting layer 5, an electron transport layer 6, an electron injection layer 7 and a cathode 8 in this order. Some of these layers may be omitted, or instead, other layers may be added. For example, a hole blocking layer may be disposed between the light-emitting layer 5 and the electron transport layer 6, or the hole transport layer 4 may be disposed directly on the anode 2 without the hole injection layer 3.

Alternatively, for example, a configuration that includes a single layer having functions of a plurality of layers, such as a single electron injection/transport layer having both the function of an electron injection layer and the function of an electron transport layer, instead of the plurality of layers is also possible. Furthermore, for example, the single hole transport layer 4 and the single electron transport layer 6 may each be composed of a plurality of layers.

### <<Layer Containing Diamantane Compound>>

In the exemplary configuration shown in FIG. 1, when the diamantane compound is contained in at least one layer selected from the group consisting of a light-emitting layer, an electron transport layer and an electron injection layer, the organic electroluminescent device 100 comprises the diamantane compound in at least one layer selected from the group consisting of the light-emitting layer 5, the electron transport layer 6 and the electron injection layer 7. Preferably, the electron transport layer 6 contains the diamantane compound. The diamantane compound may be contained in a plurality of layers of the organic electroluminescent device.

When the diamantane compound is contained in at least one layer selected from the group consisting of a light-emitting layer, a hole transport layer and a hole injection layer, the organic electroluminescent device 100 comprises the diamantane compound in at least one layer selected from the group consisting of the light-emitting layer 5, the hole transport layer 4 and the hole injection layer 3. Preferably, the hole transport layer 4 contains the diamantane compound. The diamantane compound may be contained in a plurality of layers of the organic electroluminescent device.

Hereinafter, the organic electroluminescent device 100 will be described in the context where the electron transport layer 6 contains the diamantane compound.

### [Substrate 1]

The substrate 1 may be any commonly used substrate, and examples include glass plates, quartz plates, plastic plates and plastic films. Of these, glass plates, quartz plates, and light-transmissive plastic films are preferred.

Examples of light-transmissive plastic films include films made of polyethylene terephthalate (PET), polyethylene naphthalate (PEN), polyethersulfone (PES), polyetherimide, polyether ether ketone, polyphenylene sulfide, polyarylate, polyimide, polycarbonate (PC), cellulose triacetate (TAC), cellulose acetate propionate (CAP) and the like.

In the case of a configuration in which emitted light is extracted from the substrate 1 side, the substrate 1 is transparent to the wavelength of the emitted light.

### [Anode 2]

The anode 2 is disposed on the substrate 1 (the hole injection layer 3 side).

Examples of materials of the anode include metals having a large work function (e.g., 4 eV or more), alloys, electroconductive compounds and mixtures thereof. Specific examples of the materials of the anode include metals such as Au; and conductive transparent materials such as CuI, indium tin oxide (ITO), SnO₂ and ZnO.

In the case of an organic electroluminescent device having a configuration in which emitted light is extracted through the anode, the anode is formed of a conductive transparent material that transmits or substantially transmits the emitted light.

### [Hole Injection Layer 3 and Hole Transport Layer 4]

The hole injection layer 3 and the hole transport layer 4 are disposed in this order from the anode 2 side between the anode 2 and the light-emitting layer 5 described later.

The hole injection layer 3 and the hole transport layer 4 have a function to transfer holes injected from the anode to the light-emitting layer, and the presence of the hole injection layer 3 and the hole transport layer 4 between the anode 2 and the light-emitting layer 5 allows many holes to be injected into the light-emitting layer 5 at a lower electric field.

The hole injection layer 3 and the hole transport layer 4 also function as electron barrier layers. Specifically, electrons injected from the cathode 8 and transported through the electron injection layer 7 and/or the electron transport layer 6 to the light-emitting layer 5 are prevented from leaking into the hole injection layer 3 and/or the hole transport layer 4 by an electron barrier present at the interface between the light-emitting layer 5 and the hole injection layer 3 and/or the hole transport layer 4. Consequently, the electrons are accumulated at the interface in the light-emitting layer 5 to bring benefits such as improvement in luminous efficiency, thus providing an organic electroluminescent device having excellent luminescent performance.

Materials of the hole injection layer 3 and the hole transport layer 4 have at least one of hole injectability, hole transportability, and electron barrier properties. The materials of the hole injection layer 3 and the hole transport layer 4 may be any of organic compounds and inorganic substances.

Specific examples of the materials of the hole injection layer 3 and the hole transport layer 4 include triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline copolymers, conductive polymeric oligomers (particularly, thiophene oligomers), porphyrin compounds, aromatic tertiary amine compounds and styrylamine compounds.

Of these, to provide an organic electroluminescent device with good performance, porphyrin compounds, aromatic tertiary amine compounds and styrylamine compounds are preferred, and aromatic tertiary amine compounds are particularly preferred.

Specific examples of aromatic tertiary amine compounds and styrylamine compounds include N,N,N′,N′-tetraphenyl-4,4′-diaminophenyl, N,N′-diphenyl-N,N′-bis(m-tolyl)-[1,1′-biphenyl]-4,4′-diamine (TPD), 2,2-bis(4-di-p-tolylaminophenyl)propane, 1,1-bis(4-di-p-tolylaminophenyl)cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenyl ether, 4,4′-bis(diphenylamino)quadriphenyl, N,N,N-tri(p-tolyl)amine, 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostilbenzene, N-phenylcarbazole, 4,4′-bis[N-(1-naphthyl)-N-phenylamino]biphenyl (NPD) and 4,4′,4″-tris[N-(m-tolyl)-N-phenylamino]triphenylamine (MTDATA).

Examples of the material of the hole injection layer 3 and the material of the hole transport layer 4 also include inorganic compounds such as p-type Si and p-type SiC.

The hole injection layer 3 and the hole transport layer 4 may have a single-layer structure composed of one or two or more materials, or may have a multilayer structure composed of a plurality of layers having the same composition or different compositions.

### [Light-Emitting Layer 5]

The light-emitting layer 5 is disposed between the hole transport layer 4 and the electron transport layer 6.

Examples of materials of the light-emitting layer 5, that is, luminescent materials, include phosphorescent materials, fluorescent materials and thermally activated delayed fluorescent materials. In the light-emitting layer 5, electron-hole pairs are recombined, thus resulting in light emission.

The light-emitting layer 5 may be made of a single low-molecular material or a single polymer material, but is more typically made of a host material doped with a guest compound. Light emission arises mainly from a dopant, and the light may have any color.

The host material is, for example, a compound having a biphenylyl group, a fluorenyl group, a triphenylsilyl group, a carbazole group, a pyrenyl group or an anthryl group. Specific examples include 4,4′-bis(2,2-diphenylvinyl)-1,1′-biphenyl (DPVBi), 4,4′-bis(9-ethyl-3-carbazovinylene)1,1′-biphenyl (BCzVBi), 2-tertiary butyl-9,10-di(2-naphthyl)anthracene (TBADN), 9,10-di(2-naphthyl)anthracene (ADN), 4,4′-bis(carbazol-9-yl)biphenyl (ADN), 4,4′-bis(carbazol-9-yl)-2,2′-dimethylbiphenyl (CDBP), 2-(9-phenylcarbazol-3-yl)-9-[4-(4-phenylphenylquinazolin-2-yl)carbazole and 9,10-bis(biphenyl)anthracene.

Examples of fluorescent dopants include anthracene, pyrene, tetracene, xanthene, perylene, rubrene, coumarin, rhodamine, quinacridone, dicyanomethylenepyran compounds, thiopyran compounds, polymethine compounds, pyrylium, thiapyrylium compounds, fluorene derivatives, periflanthene derivatives, indenoperylene derivatives, bis(azinyl)amine boron compounds, bis(azinyl)methane compounds, carbostyril compounds, boron compounds and cyclic amine compounds. A combination of two or more selected therefrom may be used as a fluorescent dopant.

Examples of phosphorescent dopants include metal complexes of iridium, platinum, palladium, osmium and the like.

Specific examples of the fluorescent dopants and the phosphorescent dopants include tris(8-hydroxyquinoline)aluminum (Alq3), 4,4′-bis[4-(di-p-tolylamino)styryl]biphenyl (DPAVBi), perylene, bis[2-(4-n-hexylphenyl)quinoline](acetylacetonato) iridium (III), Ir(PPy)₃(tris(2-phenylpyridine) iridium (III)) and bis(3,5-difluoro-2-(2-pyridyl)phenyl-(2-carboxypyridyl) iridium (III)) (FIrPic).

The luminescent materials are not necessarily contained only in the light-emitting layer 5. For example, the luminescent materials may be contained in a layer (the hole transport layer 4 or the electron transport layer 6) adjacent to the light-emitting layer 5. This can further enhance the luminous efficiency of the organic electroluminescent device 100.

The light-emitting layer 5 may have a single-layer structure composed of one or two or more materials, or may have a multilayer structure composed of a plurality of layers having the same composition or different compositions.

### [Electron Transport Layer 6]

The electron transport layer 6 is disposed between the light-emitting layer 5 and the electron injection layer 7.

The electron transport layer 6 has a function to transfer electrons injected from the cathode 8 to the light-emitting layer. The presence of the electron transport layer 6 between the cathode 8 and the light-emitting layer 5 allows electrons to be injected into the light-emitting layer 5 at a lower electric field.

The electron transport layer 6 preferably contains the diamantane compound, as described above. The electron transport layer 6 may further contain, in addition to the diamantane compound, one or more selected from electron transport materials known in the art.

When the diamantane compound is contained not in the electron transport layer 6 but in other layers, one or more selected from electron transport materials known in the art can be used as electron transport materials constituting the electron transport layer 6.

Examples of electron transport materials known in the art include alkali metal compounds, alkaline-earth metal compounds, transition metal compounds, zinc group element compounds and earth metal compounds. Examples of alkali metal compounds, alkaline-earth metal compounds, transition metal compounds, zinc group element compounds and earth metal compounds include chelated aryloxides such as 8-hydroxyquinolinato lithium (Liq), bis(8-hydroxyquinolinato) zinc, bis(8-hydroxyquinolinato) copper, bis(8-hydroxyquinolinato) manganese, tris(8-hydroxyquinolinato) aluminum, tris(2-methyl-8-hydroxyquinolinato) aluminum, tris(8-hydroxyquinolinato) gallium, bis(10-hydroxybenzo[h]quinolinato) beryllium, bis(10-hydroxybenzo[h]quinolinato) zinc, bis(2-methyl-8-quinolinato) chlorogallium, bis(2-methyl-8-quinolinato) (o-cresolato) gallium, bis(2-methyl-8-quinolinato)-1-naphtholato aluminum and bis(2-methyl-8-quinolinato)-2-naphtholato gallium.

The electron transport layer 6 may have a single-layer structure composed of one or two or more materials, or may have a multilayer structure composed of a plurality of layers having the same composition or different compositions.

In the organic electroluminescent device 100, the electron injection layer 7 may be disposed for the purpose of improving electron injectability and improving device characteristics (e.g., luminous efficiency, low-voltage driving or high durability).

### [Electron Injection Layer 7]

The electron injection layer 7 is disposed between the electron transport layer 6 and the cathode 8 described later.

The electron injection layer 7 has a function to transfer electrons injected from the cathode to the light-emitting layer 5. The presence of the electron injection layer between the cathode 8 and the light-emitting layer 5 allows electrons to be injected into the light-emitting layer 5 at a lower electric field.

Examples of materials of the electron injection layer 7 include organic compounds such as fluorenone, anthraquinodimethane, diphenoquinone, thiopyran dioxide, oxazole, oxadiazole, triazole, imidazole, perylenetetracarboxylic acid, fluorenylidenemethane, anthraquinodimethane and anthrone.

Examples of materials of the electron injection layer 7 also include inorganic compounds including various oxides, fluorides, nitrides and oxynitrides, such as SiO₂, AlO, SiN, SiON, AlON, GeO, LiO, LiON, TiO, TiON, TaO, TaON, TaN, LiF, C and Yb.

### [Cathode 8]

The cathode 8 is disposed on the electron injection layer 7.

In the case of an organic electroluminescence device having a configuration in which only emitted light that has passed through the anode 8 is extracted, the cathode 8 can be formed of any conductive material.

Examples of materials of the cathode 8 include metals having a small work function (hereinafter also referred to as electron injectable metals), alloys, electroconductive compounds and mixtures thereof. Here, a metal having a small work function is, for example, a metal having a work function of 4 eV or less.

Specific examples of the materials of the cathode 8 include sodium, sodium-potassium alloys, magnesium, lithium, magnesium/copper mixtures, magnesium/silver mixtures, magnesium/aluminum mixtures, magnesium/indium mixtures, aluminum/aluminum oxide (Al₂O₃) mixtures, indium, lithium/aluminum mixtures and rare earth metals.

Of these, from the viewpoint of electron injectability and resistance to, for example, oxidation, mixtures of an electron injectable metal and a second metal that is a stable metal having a work function larger than that of the electron injectable metal, such as magnesium/silver mixtures, magnesium/aluminum mixtures, magnesium/indium mixtures, aluminum/aluminum oxide (Al₂O₃) mixtures and lithium/aluminum mixtures, are preferred.

### [Hole Blocking Layer]

As described above, the organic electroluminescent device 100 may be further provided with a hole blocking layer.

Although not shown in FIG. 1, the hole blocking layer may be disposed, for example, between the light-emitting layer 5 and the electron transport layer 6.

The hole blocking layer, for example, has a function to suppress hole leakage from the light-emitting layer and increase the probability of recombination of an electron and a hole, thereby improving the luminous efficiency.

Specific examples of materials of the hole blocking layer include triazine derivatives, pyrimidine derivatives, fluoranthene derivatives, polycyclic aromatic hydrocarbon compounds, carbazole derivatives, fluorene derivatives and spirofluorene derivatives.

When the hole transport layer 4 in the organic electroluminescent device 100 contains the diamantane compound, the diamantane compound is used as the material of the hole transport layer 4 in the configuration of the organic electroluminescent device 100 described above. The hole transport layer 4 may further contain, in addition to the diamantane compound, one or more selected from hole transport materials known in the art. For the electron transport layer 6, an electron transport material known in the art can be used.

### [Method of Forming Layers]

The layers described above, excluding the electrodes (anode and cathode), can be formed by thin-film formation using, for example, a known method such as a vacuum deposition method, a spin coating method, a casting method or the LB method (Langmuir-Blodgett method). The material of each layer may be used alone or, if necessary, together with a solvent and a material such as a binder resin.

The thickness of each layer thus formed is not particularly limited and can be appropriately selected depending on the circumstances, and is typically in the range of 5 nm to 5 μm.

The anode 1 and the cathode 8 can each be formed by forming an electrode material into a thin film by a method such as vapor deposition or sputtering. In the vapor deposition or sputtering, a pattern may be formed using a mask with a desired shape, or after the thin film is formed by vapor deposition or sputtering, a pattern with a desired shape may be formed by, for example, photolithography.

The thicknesses of the anode 1 and the cathode 8 are preferably 1 μm or less, more preferably 10 nm or more and 200 nm or less.

When a layer containing the diamantane compound is formed as an electron transport layer, the diamantane compound may be used in combination with, for example, the above-described electron transport material known in the art. Thus, for example, the diamantane compound and the electron transport material known in the art may be co-deposited, or a layer of the electron transport material known in the art may be stacked on a layer of the diamantane compound.

When a layer containing the diamantane compound is formed as a hole transport layer, the diamantane compound may be used in combination with, for example, the above-described hole transport material known in the art. Thus, for example, the diamantane compound and the hole transport material known in the art may be co-deposited, or a layer of the hole transport material known in the art may be stacked on a layer of the diamantane compound.

The organic electroluminescent device according to an aspect of the present invention may be used for illumination or as a kind of lamp such as an exposure light source, or may be used as a projector from which an image is projected onto a screen or the like or a display on which a still image or a video is directly viewed.

When the organic electroluminescent device according to an aspect of the present invention is used as a display for video replay, it may be driven by a simple matrix (passive matrix) system or an active matrix system. By using two or more organic electroluminescent devices having different emission colors, a full-color display can be produced.

The diamantane compound, when used as an electron transport layer or a hole transport layer, can provide an organic electroluminescent device significantly excellent in luminous efficiency compared to when an azine compound or an amine compounds known in the art is used. Particularly when used as an electron transport layer, the diamantane compound can provide an organic electroluminescent device significantly excellent in drive voltage and luminous efficiency compared to when an azine compound known in the art is used.

Thus, effects such as improvement in drive stability and improvement in luminous efficiency of an organic electroluminescent device are expected. The diamantane compound has a low refractive index, and thus improves light extraction efficiency and contributes to improving luminous efficiency. In addition, the diamantane compound has high chemical stability due to its characteristic skeleton and can contribute to prolonging the life of an organic electroluminescent device.

The diamantane compound can provide a diamantane compound that, when used as an electron transport layer or a hole transport layer of an organic electroluminescent device, can achieve low-voltage driving and higher efficiency of the device at high levels. Furthermore, an organic electroluminescent device that is obtained using the diamantane compound and that can exhibit low-voltage driving and higher efficiency can be provided.

### EXAMPLES

The present invention will now be described in more detail with reference to Examples, but these Examples should not be construed as limiting the present invention.

### [¹H-NMR Measurement]

For the measurement of a ¹H-NMR spectrum, Bruker ASCEND400 (400 MHz; manufactured by BRUKER) was used. The ¹H-NMR spectrum was measured using deuterated chloroform (CDCl₃) as a measurement solvent and tetramethylsilane (TMS) as an internal standard substance. Commercial reagents were used.

### [DSC Measurement (Glass Transition Temperature, Crystallization Temperature and Melting Point)]

The measurements of a glass transition temperature, a crystallization temperature and a melting point were performed using DSC7020 (manufactured by Hitachi High-Tech Science Corporation, product name).

The measurement conditions of DSC are as follows. The measurement was performed in a nitrogen atmosphere (flow rate, 50 mL/min). First heating, first cooling and second heating were performed in this order, and a glass transition temperature, a crystallization temperature and a melting point determined in the second heating were respectively employed as the glass transition temperature, the crystallization temperature and the melting point of a sample.
Sample amount: 5 to 10 mg
Measurement conditions:

### <First Heating>

Heating rate: 15° C./min
Measurement temperature range: 30° C. to 360° C.

### <First Cooling>

Rapid cooling with dry ice

### <Second Heating>

Heating rate: 5° C./min
Measurement temperature range: 30° C. to 360° C.

### [Measurement of Light-Emission Characteristics]

The light-emission characteristics of an organic electroluminescent device were evaluated using a luminance meter BM-9 (product name, manufactured by TOPCON TECHNOHOUSE CORPORATION) by applying a direct current to a produced device in a 25° C. environment.

### Synthesis-1

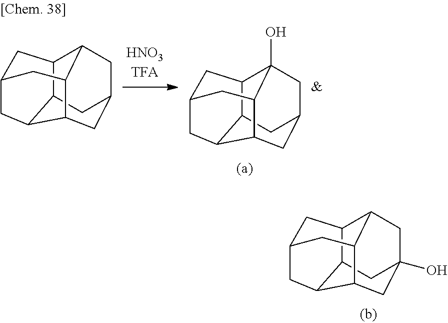

Diamantane (2.40 g, 12.8 mmol) was suspended in trifluoroacetic acid (18 mL). A 50% aqueous nitric acid solution (61 μL) was added to this suspension, and the resulting mixture was stirred at 50° C. for 16 hours. After spontaneous cooling to room temperature, the solvent was distilled off, a 10% ethanol solution of potassium hydroxide, diethyl ether and saturated saline were added, and an organic layer was separated. Sodium sulfate was added to the organic layer, and the resulting mixture was allowed to stand, after which the drying agent was filtered off, and the filtrate was concentrated under reduced pressure.

A solid that was a mixture of 1-hydroxydiamantane (a) and 4-hydroxydiamantane (b) was obtained.

The solid obtained was purified by silica gel column chromatography (hexane/ethyl acetate) and separated into 1.12 g of 1-hydroxydiamantane (white solid, 43% yield) and 0.88 g of 4-hydroxydiamantane (white solid, 36% yield). Using the compounds obtained by separation, syntheses described below were performed.

### (a) 1-Hydroxydiamantane

¹H-NMR (CDCl₃) δ (ppm): 2.16-2.18 (m, 1H), 2.13-2.15 (m, 1H), 2.03-2.08 (m, 1H), 1.94-1.97 (m, 2H), 1.72-1.76 (m, 1H), 1.61-1.67 (m, 11H), 1.45-1.47 (m, 1H), 1.41-1.44 (m, 1H).

### (b) 4-Hydroxydiamantane

¹H-NMR (CDCl₃) δ (ppm): 1.92-1.96 (m, 3H), 1.77-1.81 (m, 1H), 1.74-1.76 (m, 6H), 1.69-1.71 (m, 9H).

### Synthesis-2

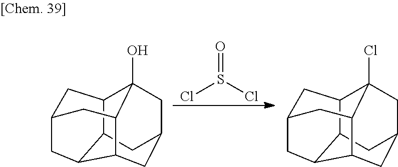

1-Hydroxydiamantane (1.69 g, 8.28 mmol) obtained in Synthesis-1 was suspended in thionyl chloride (4.5 mL, 58.0 mmol), and the suspension was stirred at room temperature for 1 hour. After the stirring, low-boiling components were distilled off under reduced pressure. The solid obtained was purified by silica gel column chromatography (hexane) to obtain 1-chlorodiamantane as a white solid (1.54 g, 84% yield).

¹H-NMR (CDCl₃) δ (ppm): 2.37-2.40 (m, 1H), 2.34-2.37 (m, 1H), 2.16-2.17 (m, 2H), 1.98-2.05 (m, 5H), 1.65-1.81 (m, 8H), 1.52-1.56 (m, 1H), 1.48-1.51 (m, 1H).

### Synthesis-3

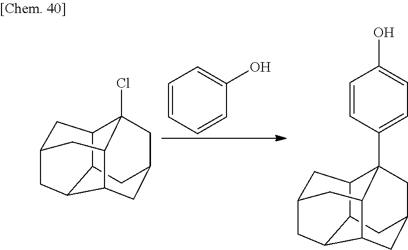

1-Chlorodiamantane (1.54 mg, 6.92 mmol) obtained in Synthesis-2 was suspended in phenol (5.21 g, 55.3 mmol), and the suspension was stirred at 120° C. for 16 hours. After the stirring, the suspension was filtered hot, and the resulting filter residue was washed with hot water. The solid obtained was purified by silica gel column chromatography (hexane/ethyl acetate=9:1) to obtain 4-(1-diamantyl) phenol as a white solid (1.81 g, 93% yield).

¹H-NMR (CDCl₃) δ (ppm): 7.21 (d, J=8.8 Hz, 2H), 6.79 (d, J=8.8 Hz, 2H), 4.50 (s, 1H), 2.28-2.30 (m, 2H), 1.96-1.98 (m, 2H), 1.75-1.89 (m, 8H), 1.63-1.68 (m, 3H), 1.52-1.53 (m, 2H), 1.41-1.45 (m, 1H), 1.38-1.42 (m, 1H).

### Synthesis-4

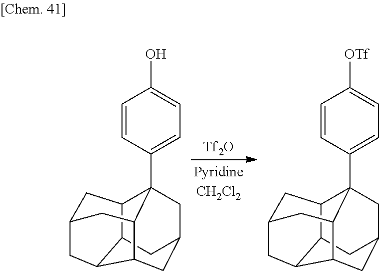 [SMILES: "Tf2O" ; Oc1ccc(C23CC4CC5C6CC(CC52)CC3C6C4)cc1 ; O=S(=O)(Oc1ccc(C23CC4CC5C6CC(CC52)CC3C6C4)cc1)C(F)(F)F]

In an argon atmosphere, 4-(1-diamantyl) phenol (1.81 g, 6.45 mmol) obtained in Synthesis-3 was suspended in dichloromethane (16 mL), and pyridine (1.53 g, 19.4 mmol) was added. This suspension was cooled 0° C., and a solution of trifluoromethanesulfonic anhydride (2.73 g, 9.68 mmol) dissolved in dichloromethane (8.0 mL) was added dropwise. After completion of the dropwise addition, the resulting mixture was stirred at 0° C. for 2 hours, and then allowed to warm to room temperature and stirred for 17 hours. After the stirring, a 1 M aqueous hydrochloric acid solution and chloroform were added, and an organic layer was separated. Sodium sulfate was added to the organic layer, and the resulting mixture was allowed to stand, after which the drying agent was filtered off, and the filtrate was concentrated under reduced pressure. The liquid obtained was purified by silica gel column chromatography (hexane) to obtain 4-(1-diamantyl)phenyl trifluoromethanesulfonate as a colorless liquid (2.32 g, 87% yield).

¹H-NMR (CDCl₃) δ (ppm): 7.41 (d, J=9.0 Hz, 2H), 7.20 (d, J=9.0 Hz, 2H), 2.30-2.33 (m, 2H), 1.99-2.01 (m, 2H), 1.88-1.92 (m, 1H), 1.68-1.81 (m, 10H), 1,51-1.52 (m, 2H), 1.43-1.49 (m, 2H).

### Synthesis-5

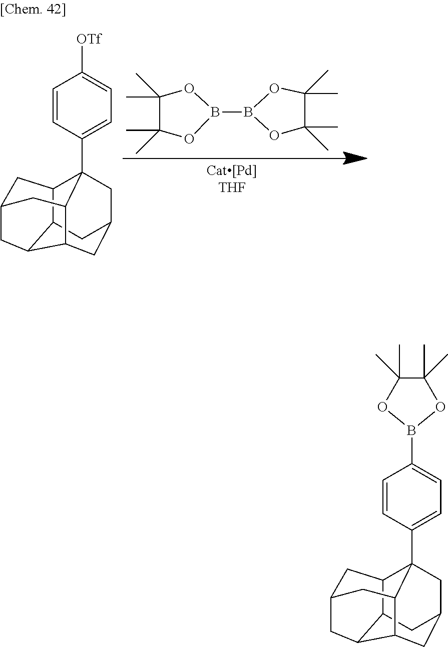

In an argon atmosphere, 4-(1-diamantyl)phenyl trifluoromethanesulfonate (3.00 g, 7.27 mmol) obtained in Synthesis-4, bispinacolatodiboron (2.03 g, 8.01 mmol), palladium acetate (0.0327 g, 0.146 mmol), 2-dicyclohexylphosphino-2′,4′,6′-triisopropylbiphenyl (0.139 g, 0.291 mol) and potassium acetate (2.14 g, 21.8 mmol) were suspended in tetrahydrofuran (73 mL), and the suspension was stirred at 70° C. for 16 hours. After spontaneous cooling to room temperature, precipitated components were removed by filtration. The filtrate was concentrated under reduced pressure and dried to obtain a crude product. Methanol was added, the resulting mixture was stirred to form a suspension, and a solid was collected by filtration. The solid obtained was dried under reduced pressure to obtain a gray powder of 4-(1-diamantyl)phenyl-4,4,5,5-tetramethyl-1,3,2-dioxaborolane (yield amount 2.8 g, 89% yield).

¹H-NMR (CDCl₃) δ (ppm): 7.78 (d, J=8.3 Hz, 2H), 7.37 (d, J=8.3 Hz, 2H), 2.36-2.40 (m, 2H), 1.96-1.99 (m, 2H), 1.87-1.89 (m, 1H), 1.63-1.82 (m, 10H), 1.49-1.53 (m, 2H), 1.40-1.46 (m, 2H), 1.34 (s, 12H).

### Synthesis-6

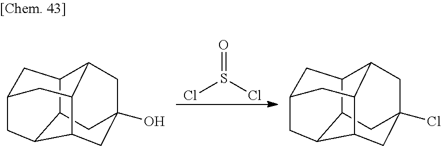

4-Hydroxydiamantane (4.50 g, 22.0 mmol) obtained in Synthesis-1 was suspended in thionyl chloride (11.9 mL, 154 mmol), and the suspension was stirred at room temperature for 1 hour. After the stirring, low-boiling components were distilled off under reduced pressure. The solid obtained was purified by silica gel column chromatography (hexane) to obtain 4-chlorodiamantane as a white solid (4.76 g, 97% yield).

¹H-NMR (CDCl₃) δ (ppm): 2.10-2.11 (m, 6H), 1.94 (s, 3H), 1.77-1.81 (m, 4H), 1.72-1.73 (m, 6H).

### Synthesis-7

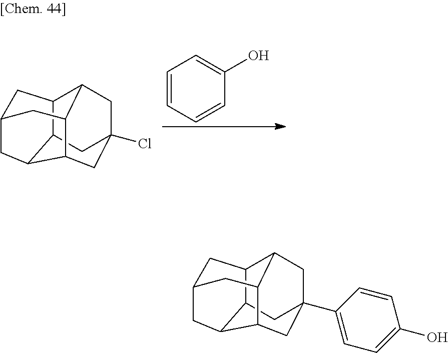

4-Chlorodiamantane (4.71 g, 21.1 mmol) obtained in Synthesis-6 was suspended in phenol (15.9 g, 169 mmol), and the suspension was stirred at 120° C. for 16 hours. After the stirring, the suspension was filtered hot, and the resulting filter residue was washed with hot water. The solid obtained was purified by silica gel column chromatography (hexane/ethyl acetate=9:1) to obtain 4-(4-diamantyl) phenol as a white solid (1.81 g, 93% yield).

¹H-NMR (CDCl₃) δ (ppm): 7.21 (d, J=8.8 Hz, 2H), 6.78 (d, J=8.8 Hz, 2H), 4.45 (s, 1H), 1.90 (s, 3H), 1.84-1.85 (m, 6H), 1.76-1.77 (m, 9H), 1.51-1.52 (m, 1H).

### Synthesis-8

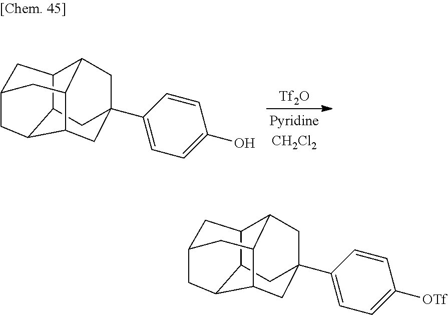 [SMILES: "Tf2O" ; Oc1ccc(C23CC4C5CC6CC4C(C2)C(C6)C5C3)cc1 ; O=S(=O)(Oc1ccc(C23CC4C5CC6CC4C(C2)C(C6)C5C3)cc1)C(F)(F)F]

In an argon atmosphere, 4-(4-diamantyl) phenol (2.00 g, 7.10 mmol) obtained in Synthesis-7 was suspended in toluene (36 mL), and pyridine (1.69 g, 21.4 mmol) was added. This suspension was cooled 0° C., and a solution of trifluoromethanesulfonic anhydride (4.02 g, 14.3 mmol) dissolved in toluene (4.0 mL) was added dropwise. After completion of the dropwise addition, the resulting mixture was stirred at 0° C. for 2 hours, and then allowed to warm to room temperature and stirred for 17 hours. After the stirring, water was added, and an organic layer was separated. Sodium sulfate was added to the organic layer, and the resulting mixture was allowed to stand, after which the drying agent was filtered off, and the filtrate was concentrated under reduced pressure. The liquid obtained was purified by silica gel column chromatography (hexane) to obtain 4-(4-diamantyl)phenyl trifluoromethanesulfonate as a colorless liquid (2.30 g, 78% yield).

¹H-NMR (CDCl₃) δ (ppm): 7.44 (d, J=8.8 Hz, 2H), 7.19 (d, J=8.8 Hz, 2H), 1.93 (s, 3H), 1.84-1.87 (m, 7H), 1.78 (d, J=2.8 Hz, 9H).

### Synthesis-9

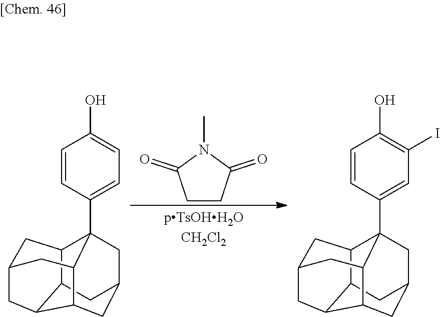

4-(1-Diamantyl) phenol (5.00 g, 17.83 mmol) and p-toluenesulfonic acid hydrate (339 mg, 1.78 mmol) were suspended in dichloromethane (90 mL), and the suspension was stirred at room temperature for 10 minutes. After the stirring, N-iodosuccinimide (4.01 g, 17.83 mmol) was added, and the resulting mixture was stirred at room temperature for 24 hours. After the stirring, a saturated aqueous sodium thiosulfate solution was added, and an organic layer was separated. Sodium sulfate was added to the organic layer, and the resulting mixture was allowed to stand, after which the drying agent was filtered off, and the filtrate was concentrated under reduced pressure. The solid obtained was purified by silica gel column chromatography (hexane/chloroform) to obtain a white solid of 2-iodo-4-(1-diamantyl) phenol (6.64 g, 92% yield).

¹H-NMR (CDCl₃) δ (ppm): 7.57 (d, J=2.3 Hz, 1H), 7.22 (dd, J=8.6, 2.3 Hz, 1H), 6.94 (d, J=8.6 Hz, 1H), 5.09 (s, 1H), 2.22-2.25 (m, 2H), 1.96-1.98 (m, 2H), 1.85-1.89 (m, 1H), 1.82 (brs, 1H), 1.79 (brs, 2H), 1.75-1.77 (m, 4H), 1.65-1.69 (m, 3H), 1.51-1.52 (m, 2H), 1.43-1.46 (m, 1H), 1.39-1.42 (m, 1H).

### Synthesis-10

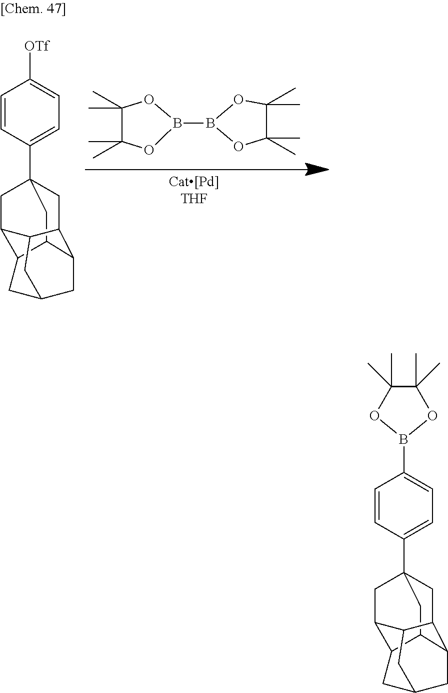

In an argon atmosphere, 4-(4-diamantyl)phenyl trifluoromethanesulfonate (6.30 g, 15.3 mmol) obtained in Synthesis-8, bispinacolatodiboron (4.27 g, 16.8 mmol), palladium acetate (0.0686 g, 0.306 mmol), 2-dicyclohexylphosphino-2′,4′,6′-triisopropylbiphenyl (0.291 g, 0.611 mol) and potassium acetate (4.50 g, 45.8 mmol) were suspended in tetrahydrofuran (153 mL), and the suspension was stirred at 70° C. for 2 hours. After spontaneous cooling to room temperature, precipitated components were removed by filtration. The filtrate was concentrated under reduced pressure and dried to obtain a crude product. Methanol was added, the resulting mixture was stirred to form a suspension, and a solid was collected by filtration. The solid obtained was dried under reduced pressure to obtain a gray powder of 4-(4-diamantyl)phenyl-4,4,5,5-tetramethyl-1,3,2-dioxaborolane (yield amount 4.5 g, 76% yield).

¹H-NMR (CDCl₃) δ (ppm): 7.77 (d, J=8.4 Hz, 2H), 7.40 (d, J=8.4 Hz, 2H), 1.88-1.91 (m, 9H), 1.77-1.78 (m, 10H), 1.33 (s, 12H).

### Synthesis Example-1

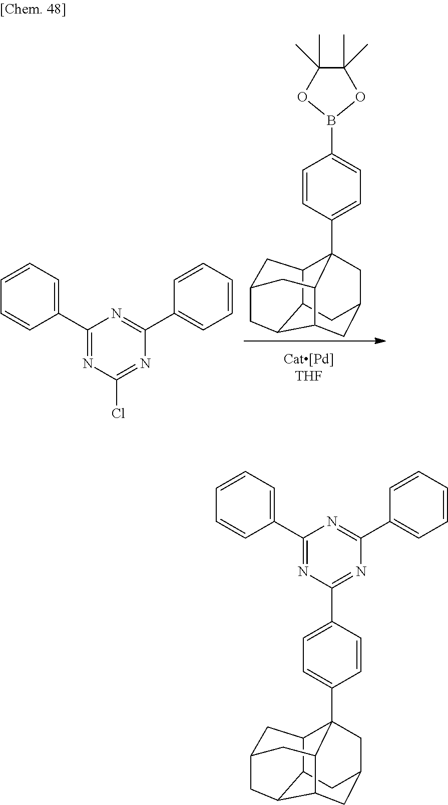

In an argon atmosphere, 2-chloro-4,6-diphenyl-1,3,5-triazine (1.50 g, 15.6 mmol), 4-(1-diamantyl)phenyl-4,4,5,5-tetramethyl-1,3,2-dioxaborolane (2.41 g, 6.16 mmol) obtained in Synthesis-5 and tetrakis(triphenylphosphine) palladium (0.194 g, 0.168 mmol) were dissolved in THF (56.0 mL). A 2 M aqueous potassium phosphate solution (8.40 mL, 16.8 mmol) was added thereto, and the resulting mixture was stirred at 70° C. for 20 hours. After spontaneous cooling to room temperature, methanol was added, and a precipitated solid was collected by filtration. The collected substance was suspended in toluene (300 mL), and the suspension was heated to 110° C. and then stirred with activated carbon added, after which Celite filtration was performed. The filtrate was concentrated and then purified by recrystallization from toluene to obtain a white solid of compound (1-6) (2.20 g, 80% yield). Its glass transition temperature was 92° C.

¹H-NMR (CDCl₃) δ (ppm): 8.77-8.79 (m, 4H), 8.71 (brd, J=8.8 Hz, 2H), 7.55-7.63 (m, 8H), 2.46 (s, 2H), 2.03 (s, 2H), 1.79-1.96 (m, 8H), 1.67-1.72 (m, 3H), 1.62 (d, J=2.4 Hz, 2H), 1.51 (s, 1H), 1.47 (s, 1H).

### Synthesis Example-2

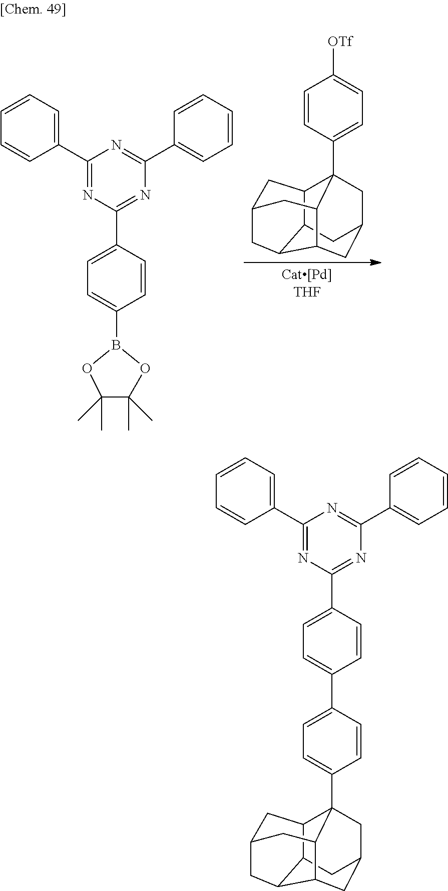 [SMILES: CC1(C)OB(c2ccc(-c3nc(-c4ccccc4)nc(-c4ccccc4)n3)cc2)OC1(C)C ; O=S(=O)(Oc1ccc(C23CC4CC5C6CC(CC52)CC3C6C4)cc1)C(F)(F)F ; c1ccc(-c2nc(-c3ccccc3)nc(-c3ccc(-c4ccc(C56CC7CC8C9CC(CC85)CC6C9C7)cc4)cc3)n2)cc1]

In an argon atmosphere, 2-[4-(4,4,5,5-tetramethyl-1,3,2-dioxaborolan-2-yl)phenyl]-4,6-diphenyl-1,3,5-triazine (2.00 g, 4.59 mmol), 4-(1-diamantyl)phenyl trifluoromethanesulfonate (2.08 g, 5.05 mmol) obtained in Synthesis-4 and tetrakis(triphenylphosphine) palladium (0.106 g, 0.092 mmol) were dissolved in THF (45.9 mL). A 2 M aqueous potassium phosphate solution (6.90 mL, 13.8 mmol) was added thereto, and the resulting mixture was stirred at 70° C. for 20 hours. After spontaneous cooling to room temperature, a precipitated solid was collected by filtration. The collected substance was suspended in toluene (300 mL), and the suspension was heated to 100° C. and then stirred with activated carbon added, after which Celite filtration was performed. The filtrate was concentrated and then purified by recrystallization from toluene to obtain a white solid of compound (1-27) (2.2 g, 84% yield). Its glass transition temperature was 131° C.

¹H-NMR (CDCl₃) δ (ppm): 8.84 (brd, J=8.0 Hz, 2H), 8.79-8.82 (m, 4H), 7.83 (brd, J=8.4 Hz, 2H), 7.69 (brd, J=8.0 Hz, 2H), 7.57-7.65 (m, 6H), 7.49 (brd, J=8.4 Hz, 2H), 2.43 (s, 2H), 2.03 (s, 2H), 1.89-1.94 (m, 3H), 1.77-1.85 (m, 5H), 1.71 (brd, J=1.6 Hz, 3H), 1.62 (d, J=2.8 Hz, 2H), 1.51 (s, 1H), 1.47 (s, 1H).

### Synthesis Example-3

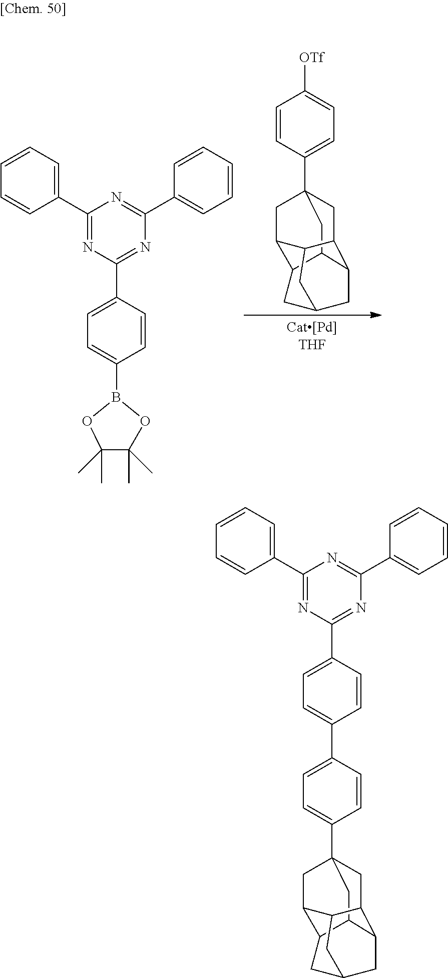

In an argon atmosphere, 2-[4-(4,4,5,5-tetramethyl-1,3,2-dioxaborolan-2-yl)phenyl]-4,6-diphenyl-1,3,5-triazine (2.00 g, 4.59 mmol), 4-(4-diamantyl)phenyl trifluoromethanesulfonate (2.08 g, 5.05 mmol) obtained in Synthesis-8 and tetrakis(triphenylphosphine) palladium (0.106 g, 0.092 mmol) were dissolved in THF (45.9 mL). A 2 M aqueous potassium phosphate solution (6.90 mL, 13.8 mmol) was added thereto, and the resulting mixture was stirred at 70° C. for 20 hours. After spontaneous cooling to room temperature, a precipitated solid was collected by filtration. The collected substance was suspended in toluene (100 mL), and the suspension was heated to 100° C. and then stirred with activated carbon added, after which Celite filtration was performed. The filtrate was concentrated and then purified by recrystallization from toluene to obtain a white solid of compound (1-87) (2.0 g, 75% yield). Its glass transition temperature was 138° C.

¹H-NMR (CDCl₃) δ (ppm): 8.84 (brd, J=8.4 Hz, 2H), 8.79-8.81 (m, 4H), 7.81 (brd, J=8.4 Hz, 2H), 7.68 (brd, J=8.4 Hz, 2H), 7.57-7.65 (m, 6H), 7.52 (brd, J=8.4 Hz, 2H), 1.95 (s, 9H), 1.77-1.85 (m, 10H).

### Synthesis Example-4

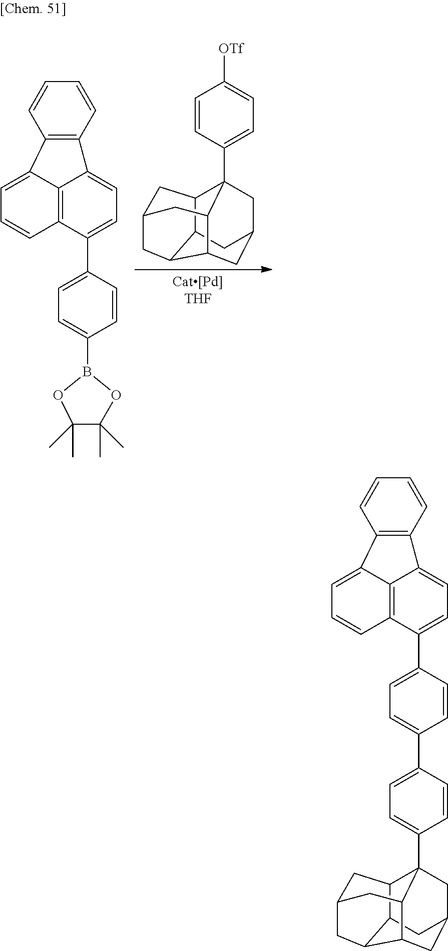

In an argon atmosphere, 4-(3-fluoranthenyl)phenyl-4,4,5,5-tetramethyl-1,3,2-dioxaborolane (1.75 g, 4.33 mmol), 4-(1-diamantyl)phenyl trifluoromethanesulfonate (1.70 g, 4.12 mmol) obtained in Synthesis-4 and tetrakis(triphenylphosphine) palladium (0.143 g, 0.124 mmol) were dissolved in THF (41.2 mL). A 2 M aqueous potassium phosphate solution (6.18 mL, 12.4 mmol) was added thereto, and the resulting mixture was stirred at 70° C. for 4 hours. After spontaneous cooling to room temperature, a precipitated solid was collected by filtration. The collected substance was suspended in toluene (600 mL), and the suspension was heated to 100° C. and then stirred with activated carbon added, after which Celite filtration was performed. The filtrate was concentrated and then purified by recrystallization from toluene to obtain a white solid of compound (1-19) (0.89 g, 40% yield). Its glass transition temperature was 131° C. ¹H-NMR (CDCl₃) δ (ppm): 8.03 (s, 1H), 8.01 (s, 1H), 7.99 (d, J=7.2 Hz, 1H), 7.92-7.96 (m, 2H), 7.75-7.79 (m, 2H), 7.63-7.69 (m, 6H), 7.48 (d, J=8.4 Hz, 2H), 7.38-7.42 (m, 2H), 2.43 (s, 2H), 2.02 (s, 2H), 1.91-1.97 (m, 3H), 1.78-1.86 (m, 5H), 1.72 (s, 3H), 1.63 (d, J=1.2 Hz, 2H), 1.51 (s, 1H), 1.47 (s, 1H).

### Synthesis Example-5

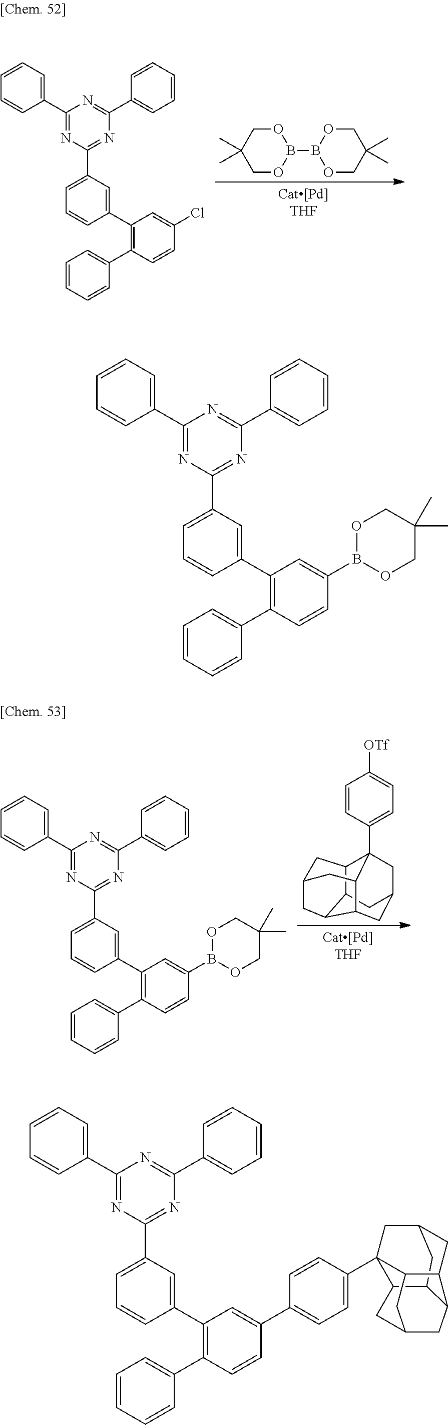

In an argon atmosphere, 4,6-diphenyl-2-[3′-chloro-1,1′:6′,1″-terphenyl-3-yl]-1,3,5-triazine (1.50 g, 3.02 mmol), bis(neopentyl glycolato)diboron (0.890 g, 3.93 mmol), palladium acetate (33.9 mg, 0.160 mmol), 2-dicyclohexylphosphino-2′,4′,6′-triisopropylbiphenyl (0.140 g, 0.300 mmol) and potassium acetate (0.890 g, 9.07 mmol) were suspended in THF (30 mL) and stirred at 80° C. for 16 hours. After spontaneous cooling to room temperature, chloroform, water and saturated saline were added, and an organic layer was separated. Sodium sulfate was added to the organic layer, and the resulting mixture was allowed to stand, after which the drying agent was filtered off, and the filtrate was concentrated under reduced pressure. The solid obtained was dissolved in chloroform, and Celite filtration was performed with activated carbon added. The filtrate was concentrated and then washed with hexane to obtain a white solid of 4,6-diphenyl-2-[3′-(5,5-dimethyl-1,3,2-dioxaborinan-2-yl)-1,1′:6′,1″-terphenyl-3-yl]-1,3,5-triazine (1.50 g, 86% yield).

¹H-NMR (CDCl₃) δ (ppm): 8.71-8.75 (m, 4H), 8.62 (s, 1H), 8.59-8.62 (m, 1H), 8.00 (d, J=0.8 Hz, 1H), 7.92 (dd, J=7.6, 0.8 Hz, 1H), 7.55-7.64 (m, 7H), 7.51 (d, J=7.6 Hz, 1H), 7.39-7.40 (m, 2H), 7.13-7.26 (m, 4H), 3.81 (s, 4H), 1.06 (s, 6H).

In an argon atmosphere, 4,6-diphenyl-2-[3′-(5,5-dimethyl-1,3,2-dioxaborinan-2-yl)-1,1′:6′,1″-terphenyl-3-yl]-1,3,5-triazine (1.50 g, 2.61 mmol), 4-(1-diamantyl)phenyl trifluoromethanesulfonate (1.29 g, 3.13 mmol) obtained in Synthesis-4, palladium acetate (12 mg, 0.05 mmol) and 2-dicyclohexylphosphino-2′,4′,6′-triisopropylbiphenyl (49.6 mg, 0.10 mmol) were dissolved in THF (26 mL). A 2 M aqueous potassium carbonate solution (1.3 mL, 2.6 mmol) was added thereto, and the resulting mixture was stirred at 80° C. for 16 hours. After spontaneous cooling to room temperature, water and methanol were added to the reaction mixture, and a precipitated solid was collected by filtration. The collected substance was dissolved in chloroform, and activated carbon was stirred in, after which Celite filtration was performed. The filtrate was concentrated and then purified by recrystallization from toluene to obtain a white solid of compound (1-54) (1.50 g, 79% yield). Its glass transition temperature was 148° C.

¹H-NMR (CDCl₃) δ (ppm): 8.72-8.75 (m, 4H), 8.68-8.69 (m, 1H), 8.64-8.66 (m, 1H), 7.82 (d, J=1.8 Hz, 1H), 7.74 (dd, J=8.0, 1.8 Hz, 1H), 7.68 (d, J=8.3 Hz, 2H), 7.55-7.64 (m, 7H), 7.46 (d, J=8.3 Hz, 2H), 7.40-7.43 (m, 2H), 7.23-7.31 (m, 4H), 7.15-7.19 (m, 1H), 2.39-2.42 (m, 2H), 1.99-2.04 (m, 2H), 1.88-1.93 (m, 3H), 1.76-1.85 (m, 5H), 1.68-1.71 (m, 3H), 1.60-1.62 (m, 2H), 1.44-1.49 (m, 2H).

### Synthesis Example-6

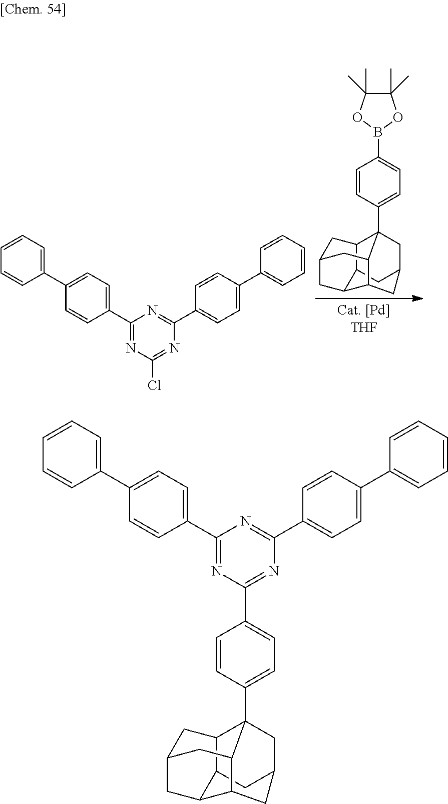

In an argon atmosphere, 2-chloro-4,6-bisbiphenylyl-1,3,5-triazine (2.00 g, 4.76 mmol), 4-(1-diamantyl)phenyl-4,4,5,5-tetramethyl-1,3,2-dioxaborolane (2.23 g, 5.72 mmol) obtained in Synthesis-5 and tetrakis(triphenylphosphine) palladium (0.550 g, 0.476 mmol) were dissolved in THF (48 mL). A 2 M aqueous potassium carbonate solution (7.10 mL, 14.3 mmol) was added thereto, and the resulting mixture was stirred at 80° C. for 15 hours. After spontaneous cooling to room temperature, methanol was added, and a precipitated solid was collected by filtration. The collected substance was suspended in chloroform, and the suspension was heated to 110° C. and then stirred with activated carbon added, after which Celite filtration was performed. The filtrate was concentrated and then purified by recrystallization from toluene to obtain a white solid of compound (1-8) (1.88 g, 61% yield). Its glass transition temperature was 147° C.

¹H-NMR (CDCl₃) δ (ppm): 8.87 (d, J=8.6 Hz, 4H), 8.74 (d, J=8.6 Hz, 2H), 7.82 (d, J=8.6 Hz, 4H), 7.71-7.74 (m, 4H), 7.60 (d, J=8.6 Hz, 2H), 7.49-7.53 (m, 4H), 7.40-7.44 (m, 2H), 2.45-2.49 (m, 2H), 2.02-2.06 (m, 2H), 1.93-1.97 (m, 1H) 1.79-1.91 (m, 7H), 1.68-1.74 (m, 3H), 1.63-1.64 (m, 2H), 1.52-1.55 (m, 1H), 1.47-1.50 (m, 1H).

### Synthesis Example-7

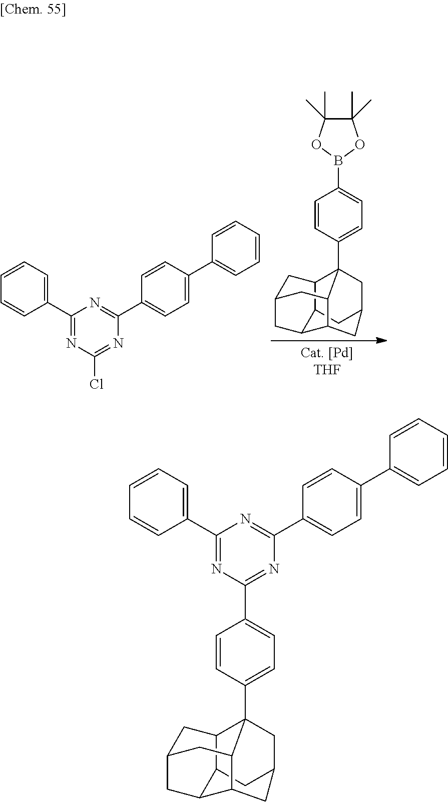

In an argon atmosphere, 2-chloro-4-biphenylyl-6-phenyl-1,3,5-triazine (2.00 g, 5.82 mmol), 4-(1-diamantyl)phenyl-4,4,5,5-tetramethyl-1,3,2-dioxaborolane (2.73 g, 6.98 mmol) and tetrakis(triphenylphosphine) palladium (0.336 g, 0.291 mmol) were dissolved in tetrahydrofuran (58 mL). A 2 M aqueous potassium carbonate solution (8.70 mL, 17.45 mmol) was added thereto, and the resulting mixture was stirred at 80° C. for 15 hours. After spontaneous cooling to room temperature, methanol was added, and a precipitated solid was collected by filtration. The collected substance was dissolved in chloroform, and the resulting solution was stirred with activated carbon added, after which Celite filtration was performed. The filtrate was concentrated and then purified by recrystallization from toluene to obtain a white solid of compound (1-9) (1.69 g, 51% yield). Its glass transition temperature was 118° C.

¹H-NMR (CDCl₃) δ (ppm): 8.85 (d, J=8.6 Hz, 2H), 8.79-8.81 (m, 2H), 8.73 (d, J=8.6 Hz, 2H), 7.8 (d, J=8.6 Hz, 2H), 7.70-7.73 (m, 2H), 7.57-7.62 (m, 5H), 7.48-7.53 (m, 2H), 7.40-7.44 (m, 1H), 2.44-2.49 (m, 2H), 2.02-2.06 (m, 2H), 1.93-1.96 (m, 1H), 1.78-1.90 (m, 7H), 1.68-1.71 (m, 3H), 1.63-1.64 (m, 2H), 1.51-1.53 (m, 1H), 1.48-1.50 (m, 1H).

### Synthesis Example-8

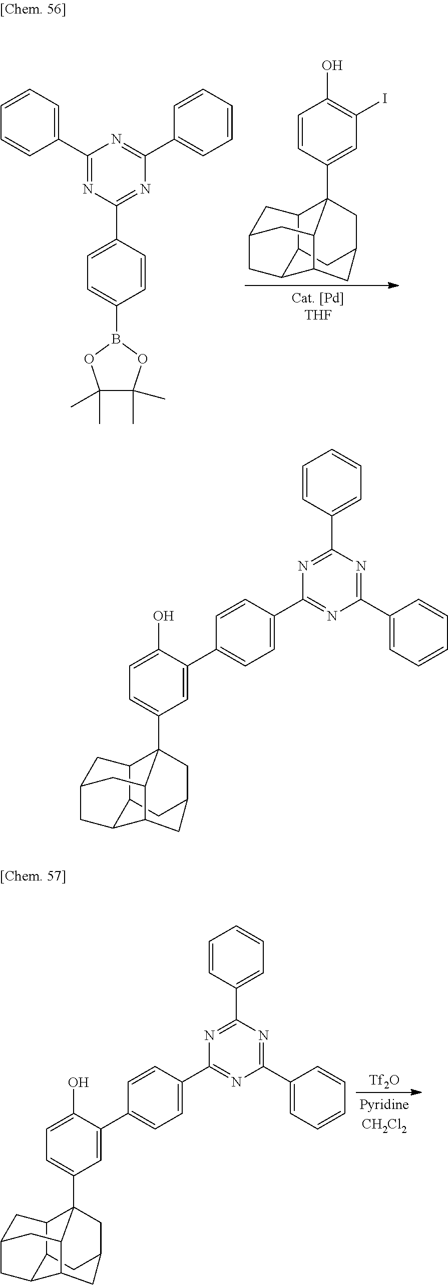 [SMILES: CC1(C)OB(c2ccc(-c3nc(-c4ccccc4)nc(-c4ccccc4)n3)cc2)OC1(C)C ; Oc1ccc(C23CC4CC5C6CC(CC52)CC3C6C4)cc1I ; "Tf2O" ; Oc1ccc(C23CC4CC5C6CC(CC52)CC3C6C4)cc1-c1ccc(-c2nc(-c3ccccc3)nc(-c3ccccc3)n2)cc1 ; Oc1ccc(C23CC4CC5C6CC(CC52)CC3C6C4)cc1-c1ccc(-c2nc(-c3ccccc3)nc(-c3ccccc3)n2)cc1]

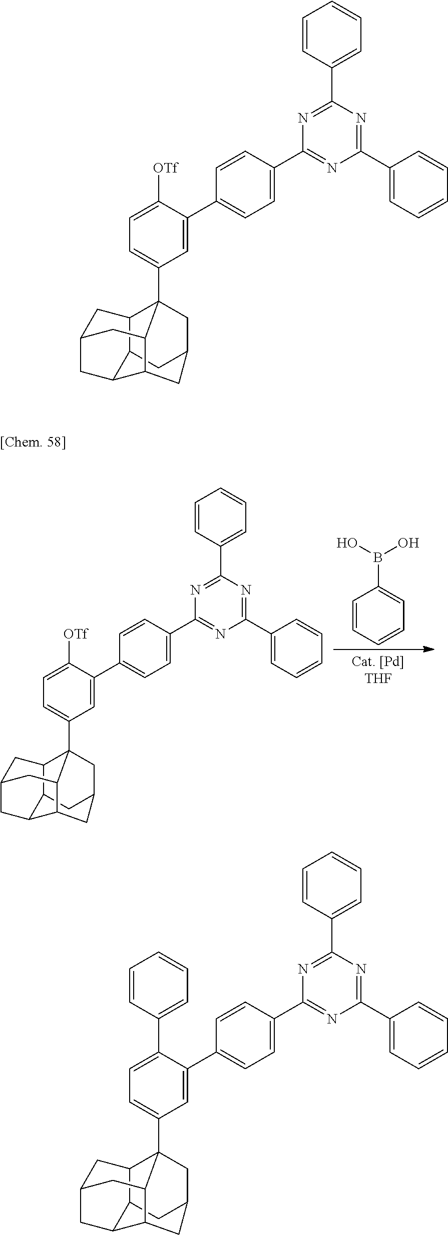

In an argon atmosphere, 2-[4-(4,4,5,5-tetramethyl-1,3,2-dioxaborolan-2-yl)phenyl]-4,6-diphenyl-1,3,5-triazine (3.86 g, 8.86 mmol), 2-iodo-4-(1-diamantyl) phenol (3.00 g, 7.38 mmol), palladium acetate (83 mg, 0.37 mmol) and tricyclohexylphosphine (124 mg, 0.44 mmol) were dissolved in tetrahydrofuran (73.8 mL). A 2 M aqueous potassium carbonate solution (11.1 mL, 22.15 mmol) was added thereto, and the resulting mixture was stirred at 80° C. for 16 hours. After spontaneous cooling to room temperature, water was added, and an organic layer was separated. Sodium sulfate was added to the organic layer, and the resulting mixture was allowed to stand, after which the drying agent was filtered off, and the filtrate was concentrated under reduced pressure. The collected substance was dissolved in chloroform (250 mL), and the resulting solution was stirred with activated carbon added, after which Celite filtration was performed. After the filtrate was concentrated, the solid obtained was purified by silica gel column chromatography (hexane/chloroform) to obtain a white solid of 4,6-diphenyl-2-[2′-hydroxy-4′-(1-diamantyl)-1,1′-biphenyl-4-yl]-1,3,5-triazine (2.0 g, 75% yield). Its glass transition temperature was 147° C.

¹H-NMR (CDCl₃) δ (ppm): 8.89 (d, J=8.5 Hz, 2H), 8.79-8.81 (m, 4H), 7.73 (d, J=8.5 Hz, 2H), 7.57-7.66 (m, 6H), 7.28-7.31 (m, 2H), 6.98 (d, J=8.3 Hz, 1H), 5.07 (brs, 1H), 2.34-2.37 (m, 2H), 1.98-2.02 (m, 2H), 1.95 (brs, 1H), 1.90-1.92 (m, 2H), 1.75-1.83 (m, 5H), 1.68-1.72 (m, 3H), 1.61-1.62 (m, 2H), 1.44-1.49 (m, 2H).

In an argon atmosphere, 4,6-diphenyl-2-[2′-hydroxy-4′-(4-diamantyl)-1,1′-biphenyl-4-yl]-1,3,5-triazine (2.86 g, 4.86 mmol) was suspended in dichloromethane 24.3 mL), and pyridine (1.54 g, 19.43 mmol) was added. This suspension was cooled 0° C., and trifluoromethanesulfonic anhydride (2.74 g, 9.71 mmol) was added dropwise. After completion of the dropwise addition, the resulting mixture was stirred at 0° C. for 2 hours, and then allowed to warm to room temperature and stirred for 17 hours. After the stirring, water was added, and an organic layer was separated. Sodium sulfate was added to the organic layer, and the resulting mixture was allowed to stand, after which the drying agent was filtered off, and the filtrate was concentrated under reduced pressure. The liquid obtained was purified by silica gel column chromatography (hexane) to obtain a white solid of 4,6-diphenyl-2-[2′-trifluoromethanesulfonate-4′-(1-diamantyl)-1,1′-biphenyl-4-yl]-1,3,5-triazine (3.09 g, 88% yield).

¹H-NMR (CDCl₃) δ (ppm): 8.88 (d, J=8.4 Hz, 2H), 8.79-8.81 (m, 4H), 7.69 (d, J=8.4 Hz, 2H), 7.57-7.66 (m, 6H), 7.50 (d, J=2.4 Hz, 1H), 7.45 (dd, J=8.6, 2.4 Hz, 1H), 7.37 (d, J=8.6 Hz, 1H), 2.37-2.40 (m, 2H), 2.00-2.04 (m, 2H), 1.92-1.96 (m, 1H), 1.80-1.84 (m, 6H), 1.70-1.74 (m, 3H), 1.61-1.62 (m, 2H), 1.48-1.52 (m, 2H).

In an argon atmosphere, 4,6-diphenyl-2-[2′-trifluoromethanesulfonate-4′-(1-diamantyl)-1,1′-biphenyl-4-yl]-1,3,5-triazine (2.89 g, 4.01 mmol), phenylboronic acid (1.96 g, 16.06 mmol) and tetrakis(triphenylphosphine) palladium (0.928 g, 0.80 mmol) were dissolved in tetrahydrofuran (12 mL). A 2 M aqueous potassium carbonate solution (12.05 mL, 24.09 mmol) was added thereto, and the resulting mixture was stirred at 80° C. for 15 hours. After spontaneous cooling to room temperature, methanol was added, and a precipitated solid was collected by filtration. The collected substance was dissolved in chloroform, and the resulting solution was stirred with activated carbon added, after which Celite filtration was performed. The filtrate was concentrated and then purified by recrystallization from toluene to obtain a white solid of compound (1-103) (1.77 g, 68% yield).

¹H-NMR (CDCl₃) δ (ppm): 8.75-8.78 (m, 4H), 8.65 (d, J=8.6 Hz, 2H), 7.55-7.63 (m, 6H), 7.47 (brs, 1H), 7.43-7.45 (m, 2H), 7.38 (d, J=8.6 Hz, 2H), 7.22 (brs, 5H), 2.44-2.47 (m, 2H), 2.00-2.03 (m, 3H), 1.98 (brs, 1H), 1.93-1.95 (m, 1H), 1.77-1.86 (m, 5H), 1.72-1.75 (m, 3H), 1.68-1.69 (m, 2H), 1.48-1.54 (m, 2H).

### Synthesis Example-9

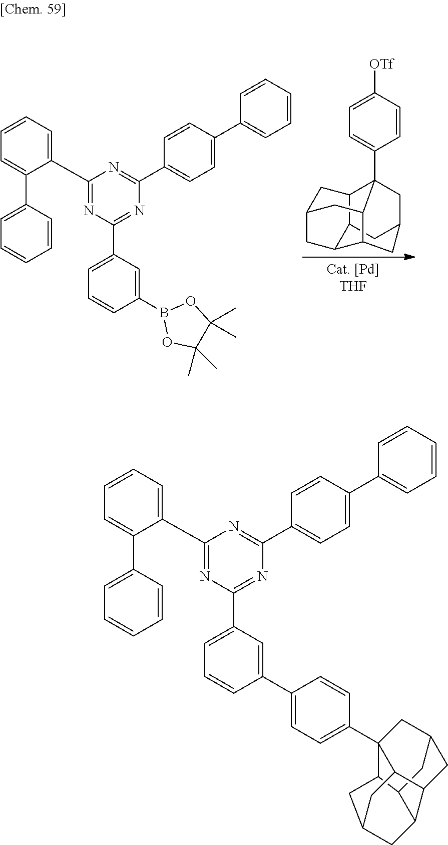 [SMILES: CC1(C)OB(c2cccc(-c3nc(-c4ccc(-c5ccccc5)cc4)nc(-c4ccccc4-c4ccccc4)n3)c2)OC1(C)C ; O=S(=O)(Oc1ccc(C23CC4CC5C6CC(CC52)CC3C6C4)cc1)C(F)(F)F ; c1ccc(-c2ccc(-c3nc(-c4cccc(-c5ccc(C67CC8CC9C%10CC(CC96)CC7C%10C8)cc5)c4)nc(-c4ccccc4-c4ccccc4)n3)cc2)cc1]

In an argon atmosphere, 2-[1-(4,4,5,5-tetramethyl-1,3,2-dioxaborolane)phenyl-3-yl]-4-(1,1′-biphenyl-2-yl)-6-biphenylyl-1,3,5-triazine (2.22 g, 3.78 mmol), 4-(1-diamantyl)phenyl trifluoromethanesulfonate (1.30 g, 3.15 mmol), palladium acetate (0.035 g, 0.158 mmol) and 2-dicyclohexylphosphino-2′,4′,6′-triisopropylbiphenyl (150.2 mg, 0.315 mmol) were dissolved in tetrahydrofuran (32 mL). A 2 M aqueous tripotassium phosphate solution (4.73 mL, 9.46 mmol) was added thereto, and the resulting mixture was stirred at 80° C. for 15 hours. After spontaneous cooling to room temperature, methanol was added, and a precipitated solid was collected by filtration. The collected substance was dissolved in chloroform, and the resulting solution was stirred with activated carbon added, after which Celite filtration was performed. The filtrate was concentrated and then purified by recrystallization from toluene to obtain a white solid of compound (1-104) (1.64 g, 72% yield). Its glass transition temperature was 125° C.

¹H-NMR (CDCl₃) δ (ppm): 8.60 (t, J=1.7 Hz, 1H), 8.41 (d, J=8.5 Hz, 2H), 8.36 (dd, J=7.3, 1.7 Hz, 1H), 8.31-8.33 (m, 1H), 7.77-7.80 (m, 1H), 7.67-7.70 (m, 4H), 7.47-7.65 (m, 10H), 7.38-7.43 (m, 1H), 7.35-7.38 (m, 2H), 7.29-7.33 (m, 2H), 7.18-7.23 (m, 1H), 2.43-2.46 (m, 2H), 2.01-2.05 (m, 2H), 1.92-1.95 (m, 3H), 1.78-1.87 (m, 5H), 1.70-1.74 (m, 3H), 1.64-1.65 (m, 2H), 1.51-1.53 (m, 1H), 1.48-1.50 (m, 1H).

### Synthesis Example-10

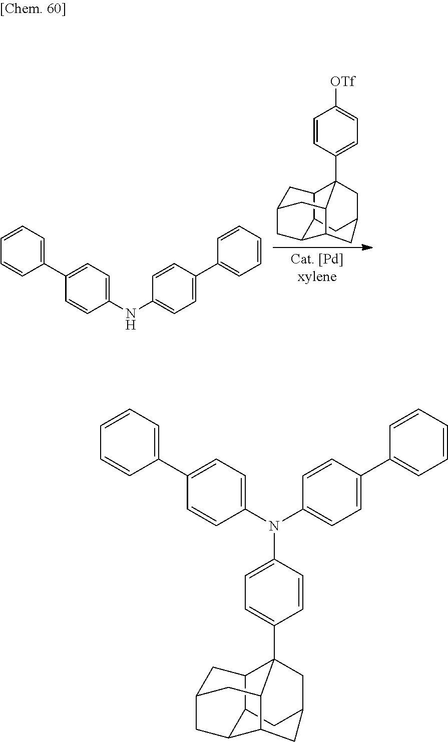

In an argon atmosphere, 4-(4-diamantyl)phenyl trifluoromethanesulfonate (1.78 g, 4.30 mmol), bis(4-biphenylyl)amine (1.66 g, 5.16 mmol), trisdibenzylideneacetone dipalladium (118 mg, 0.13 mol), 2-dicyclohexylphosphino-2′,6′-dimethoxybiphenyl (106 mg, 0.26 mmol) and sodium-tert-butoxide (620 mg, 6.45 mmol) were suspended in xylene (17 mL), and the suspension was stirred at 140° C. for 40 hours. After spontaneous cooling to room temperature, toluene was added to the reaction mixture, and the resulting mixture was filtered through Celite. The solvent was distilled off, and the crude product obtained was purified by silica gel column chromatography (hexane/chloroform) to obtain a white powder of compound (1-106) (1.61 g, 64% yield). Its glass transition temperature was 104° C.

¹H-NMR (CDCl₃) δ (ppm): 7.57-7.60 (m, 4H), 7.50 (d, J=8.4 Hz, 4H), 7.40-7.44 (m, 4H), 7.28-7.33 (m, 2H), 7.25 (d, J=8.4 Hz, 2H), 7.19 (d, J=8.4 Hz, 4H), 7.12 (d, J=8.4 Hz, 2H), 2.30-7.35 (m, 2H), 1.97-2.01 (m, 2H), 1.88-1.95 (m, 3H), 1.76-1.83 (m, 5H), 1.69-1.72 (m, 3H), 1.59-1.60 (m, 2H), 1.42-1.48 (m, 2H).

### Synthesis Example-11

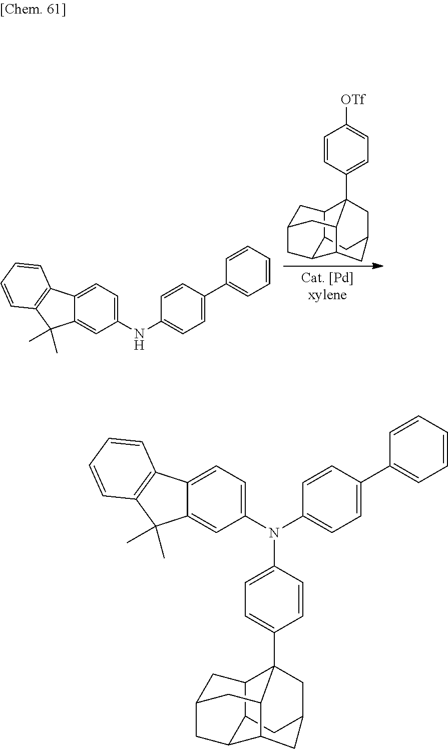

In an argon atmosphere, 4-(4-diamantyl)phenyl trifluoromethanesulfonate (2.06 g, 5.00 mmol), 2-(1,1′-biphenyl-4-yl)amino-9,9-dimethylfluorene (2.17 g, 6.00 mmol), trisdibenzylideneacetone dipalladium (137 mg, 0.15 mol), 2-dicyclohexylphosphino-2′,6′-dimethoxybiphenyl (123 mg, 0.30 mmol) and sodium-tert-butoxide (721 mg, 7.50 mmol) were suspended in xylene (20 mL), and the suspension was stirred at 140° C. for 40 hours. After spontaneous cooling to room temperature, toluene was added to the reaction mixture, and the resulting mixture was filtered through Celite. The solvent was distilled off, and the crude product obtained was purified by silica gel column chromatography (hexane/chloroform) to obtain a white powder of compound (1-113) (1.94 g, 61% yield). Its glass transition temperature was 116° C.

¹H-NMR (CDCl₃) δ (ppm): 7.64 (d, J=7.6 Hz, 1H), 7.58-7.60 (m, 3H), 7.49 (d, J=8.6 Hz, 2H), 7.38-7.44 (m, 3H), 7.29-7.34 (m, 2H), 7.22-7.28 (m, 4H), 7.19 (d, J=8.6 Hz, 2H), 7.12 (d, J=8.4 Hz, 2H), 7.06-7.09 (m, 1H), 2.30-2.34 (m, 2H), 1.96-2.00 (m, 2H), 1.88-1.95 (m, 3H), 1.74-1.83 (m, 5H), 1.69-1.72 (m, 3H), 1.59-1.60 (m, 2H), 1.42-1.47 (m, 8H).

### Synthesis Example-12

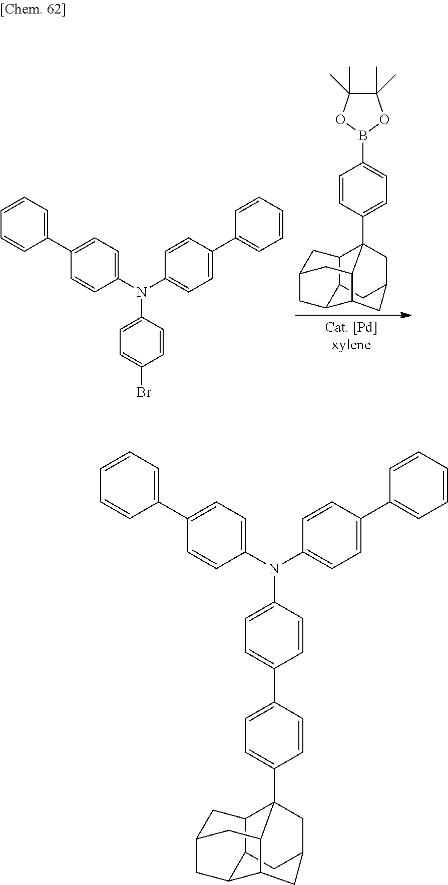

In an argon atmosphere, N,N-bis(1,1′-biphenyl-4-yl)-N-(4-bromophenyl)amine (2.37 g, 4.96 mmol), 4-(1-diamantyl)phenyl-4,4,5,5-tetramethyl-1,3,2-dioxaborolane (2.13 g, 5.46 mmol) and tetrakis(triphenylphosphine) palladium (0.287 g, 0.248 mmol) were dissolved in 1,4-dioxane (50 mL). A 2 M aqueous potassium carbonate solution (7.44 mL, 14.88 mmol) was added thereto, and the resulting mixture was stirred at 110° C. for 18 hours. After the stirring, water was added, and an organic layer was separated. Sodium sulfate was added to the organic layer, and the resulting mixture was allowed to stand, after which the drying agent was filtered off, and the filtrate was concentrated under reduced pressure. The solid obtained was dissolved in chloroform, and the resulting solution was stirred with activated carbon added, after which Celite filtration was performed. The filtrate was concentrated and then purified by recrystallization from toluene to obtain a white solid of compound (1-108) (2.65 g, 81% yield). Its glass transition temperature was 129° C.

¹H-NMR (CDCl₃) δ (ppm): 7.51-7.61 (m, 12H), 7.41-7.45 (m, 6H), 7.30-7.34 (m, 2H), 7.22-7.25 (m, 6H), 2.38-2.41 (m, 2H), 1.99-2.02 (m, 2H), 1.86-1.93 (m, 3H), 1.76-1.85 (m, 5H), 1.67-1.71 (m, 3H), 1.59-1.60 (m, 2H), 1.45-1.48 (m, 2H).

### Synthesis Example-13

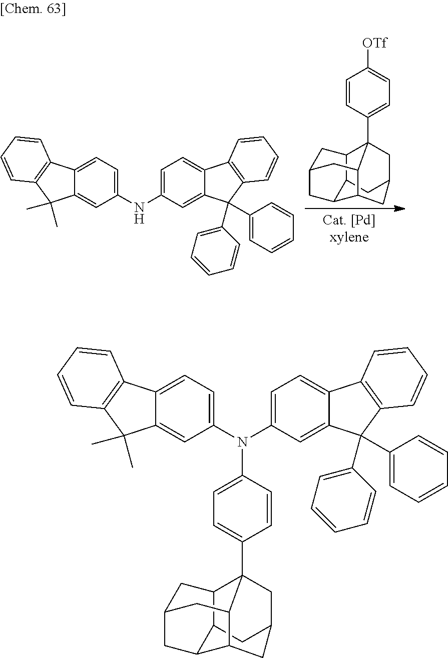

In an argon atmosphere, 4-(4-diamantyl)phenyl trifluoromethanesulfonate (2.06 g, 5.00 mmol), N-(9,9-dimethyl-9H-fluoren-2-yl)-9,9-diphenyl-9H-fluorene-2-amine (3.15 g, 6.00 mmol), trisdibenzylideneacetone dipalladium (137 mg, 0.15 mol), 2-dicyclohexylphosphino-2′,6′-dimethoxybiphenyl (123 mg, 0.30 mmol) and sodium-tert-butoxide (721 mg, 7.50 mmol) were suspended in xylene (20 mL), and the suspension was stirred at 140° C. for 40 hours. After spontaneous cooling to room temperature, toluene was added to the reaction mixture, and the resulting mixture was filtered through Celite. The solvent was distilled off, and the crude product obtained was purified by silica gel column chromatography (hexane/chloroform) to obtain a white powder of compound (1-117) (2.69 g, 68% yield). Its glass transition temperature was 151° C. ¹H-NMR (CDCl₃) δ (ppm): 7.66-7.68 (m, 1H), 7.60-7.62 (m, 2H), 7.52 (d, J=8.3 Hz, 1H), 7.36-7.38 (m, 1H), 7.27-7.33 (m, 3H), 7.21-7.24 (m, 2H), 7.12-7.21 (m, 14H), 7.09 (dd, J=8.3, 2.0 Hz, 1H), 6.97-7.01 (m, 3H), 2.28-2.31 (m, 2H), 1.96-1.99 (m, 2H), 1.85-1.91 (m, 3H), 1.74-1.82 (m, 5H), 1.68-1.71 (m, 3H), 1.57-1.58 (m, 2H), 1.41-1.46 (m, 2H), 1.35 (s, 6H).

### Synthesis Example-14

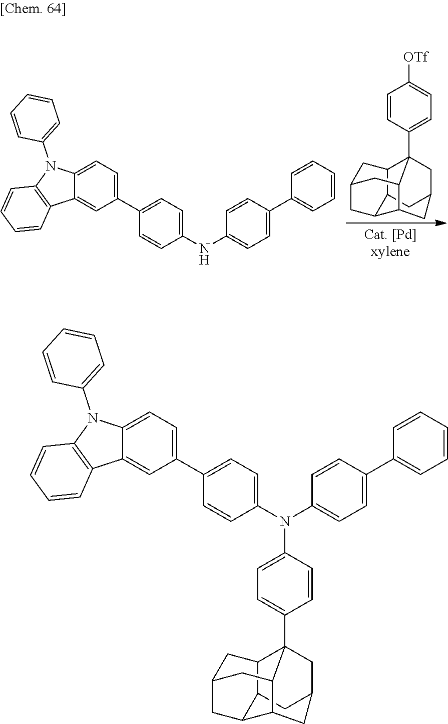

In an argon atmosphere, 4-(4-diamantyl)phenyl trifluoromethanesulfonate (2.06 g, 5.00 mmol), 3-[4-(4-biphenylylamino)phenyl]-9-phenylcarbazole (2.92 g, 6.00 mmol), trisdibenzylideneacetone dipalladium (137 mg, 0.15 mol), 2-dicyclohexylosph ino-2′,6′-dimethoxybiphenyl (123 mg, 0.30 mmol) and sodium-tert-butoxide (721 mg, 7.50 mmol) were suspended in xylene (20 mL), and the suspension was stirred at 140° C. for 40 hours. After spontaneous cooling to room temperature, toluene was added to the reaction mixture, and the resulting mixture was filtered through Celite. The solvent was distilled off, and the crude product obtained was purified by silica gel column chromatography (hexane/chloroform) to obtain a white powder of compound (1-118) (yield amount 2.52 g, 67% yield). Its glass transition temperature was 144° C.

¹H-NMR (CDCl₃) δ (ppm): 8.34 (s, 1H), 8.18 (d, J=7.7 Hz, 1H), 7.57-7.67 (m, 10H), 7.47-7.52 (m, 4H), 7.39-7.46 (m, 5H), 7.20-7.34 (m, 4H), 7.14-7.19 (m, 4H), 2.32-2.35 (m, 2H), 1.97-2.00 (m, 2H), 1.89-1.96 (m, 3H), 1.77-1.83 (m, 5H), 1.69-1.72 (m, 3H), 1.60-1.61 (m, 2H), 1.43-1.49 (m, 2H).

### Synthesis Example-15

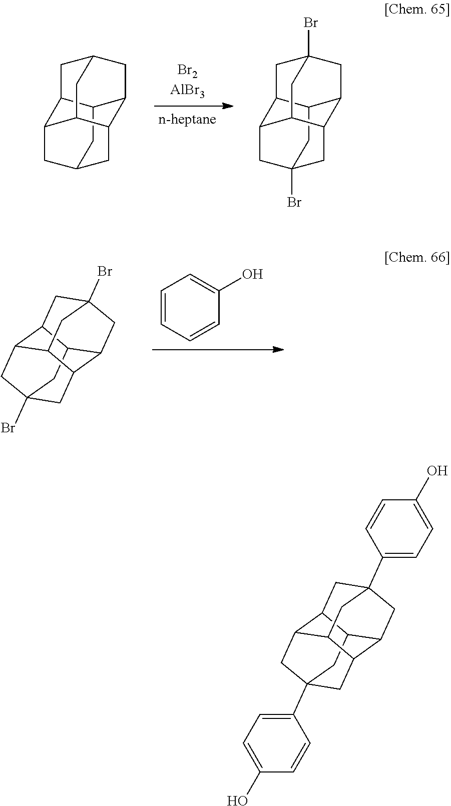

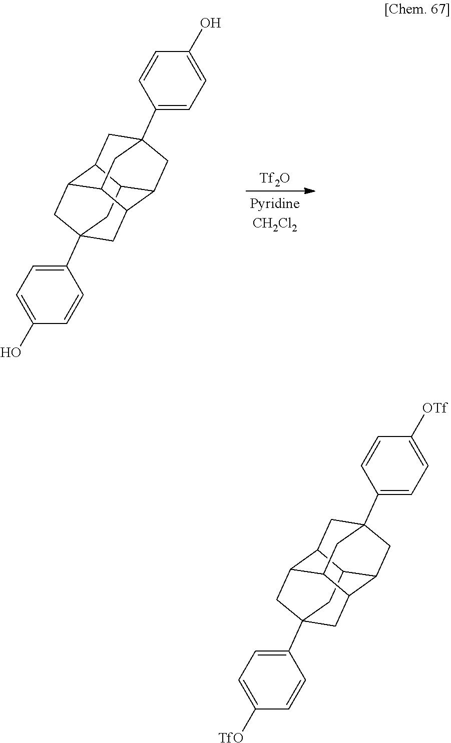 [SMILES: "Tf2O" ; Oc1ccc(C23CC4C5CC6(c7ccc(O)cc7)CC4C(C2)C(C6)C5C3)cc1 ; O=S(=O)(Oc1ccc(C23CC4C5CC6(c7ccc(OS(=O)(=O)C(F)(F)F)cc7)CC4C(C2)C(C6)C5C3)cc1)C(F)(F)F]

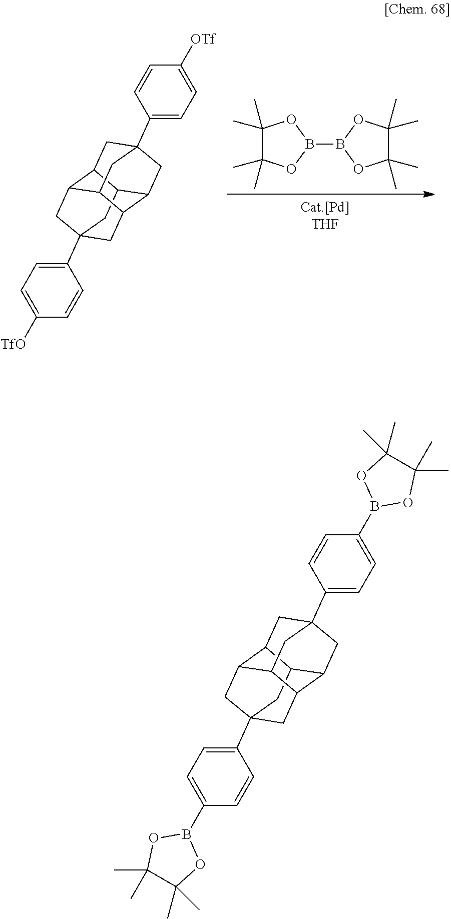

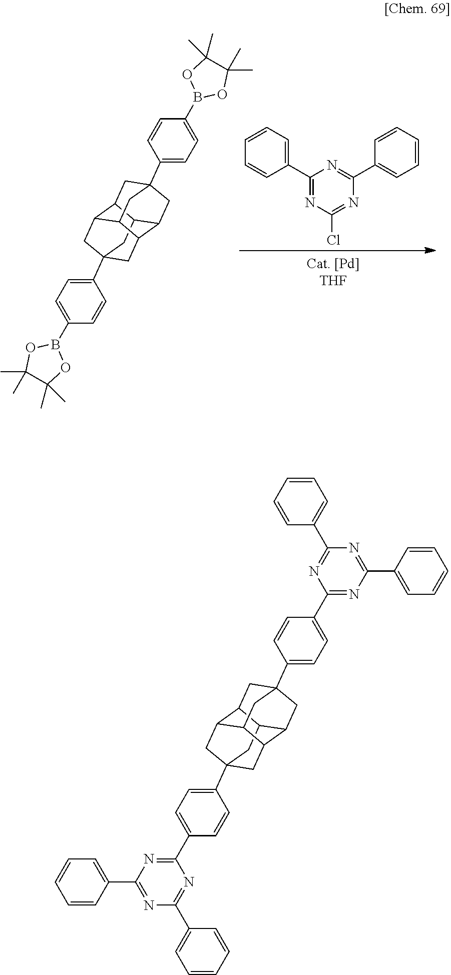

In an argon atmosphere, congressane (4.16 g, 22.07 mmol) and aluminum bromide (0.46 g, 1.72 mmol) were suspended in heptane (22.0 ml). To the suspension cooled to −10° C., a solution obtained by dissolving bromine (17.99 g, 112.56 mmol) in heptane (10.4 ml) was added dropwise over 2 hours. After the dropwise addition, the temperature was raised to 0° C., and after stirring for 1 hour, the resulting mixture was transferred to an ice bath using carbon tetrachloride (24 ml), and sodium hydrogen carbonate was added. Water and dichloromethane were added, and an organic layer was separated. Sodium sulfate was added to the organic layer, and the resulting mixture was allowed to stand, after which the drying agent was filtered off, and the filtrate was concentrated under reduced pressure. The solid obtained was purified by recrystallization from acetone to obtain 3,7-dibromodiamantane as a white solid (3.07 g, 40% yield).

¹H-NMR (CDCl₃) δ (ppm): 2.32 (brs, 12H), 1.97 (brs, 6H).

3,7-Dibromodiamantane (3.01 g, 8.70 mmol) was suspended in phenol (8.18 g, 86.97 mmol), and the suspension was stirred at 120° C. for 116 hours. After the stirring, the suspension was filtered hot, and the resulting filter residue was washed with hot water. The solid obtained was purified by recrystallization from methanol to obtain 3,7-di(1-hydroxyphenyl-4-yl)diamantane as a white solid (3.09 g, 95% yield).

¹H-NMR (DMSO-d6) δ (ppm): 7.17 (d, J=8.6 Hz, 4H), 6.69 (d, J=8.6 Hz, 4H), 1.88 (brs, 6H), 1.83 (brs, 12H).

In an argon atmosphere, 3,7-di(1-hydroxyphenyl-4-yl)diaman (3.09 g, 8.28 mmol) was suspended in methylene chloride (41.4 ml). Pyridine (5.24 g, 66.25 mmol) was added to this suspension, and the resulting mixture was cooled to 0° C. Trifluoromethanesulfonic anhydride (9.35 g, 33.13 mmol) was added dropwise, and the resulting mixture was stirred for 4 hours. After the consumption of the raw materials was confirmed by TLC, the mixture was quenched with hydrochloric acid and extracted with chloroform. The organic layer obtained was washed with a saturated aqueous sodium hydrogen carbonate solution and dried over sodium sulfate to distill off the solvent. The crude product obtained was purified by silica gel column chromatography (hexane/ethyl acetate) to obtain 3,7-di(1-trifluoromethanesulfonylphenyl-4-yl)diamantane as a white solid (4.91 g, 93% yield).

¹H-NMR (CDCl₃) δ (ppm): 7.46 (d, J=8.9 Hz, 4H), 7.22 (d, J=8.9 Hz, 4H), 1.99 (brs, 6H), 1.95 (brs, 12H).

In an argon atmosphere, 3,7-di(1-trifluoromethanesulfonylphenyl-4-yl)diamantane (2.00 g, 3.14 mmol), bispinacolatodiboron (1.91 g, 7.54 mmol), palladium acetate (0.07 g, 0.31 mmol), 2-dicyclohexylphosphino-2′,4′,6′-triisopropylbiphenyl (0.30 g, 0.63 mol) and potassium acetate (1.85 g, 18.85 mmol) were suspended in tetrahydrofuran (31 mL), and the suspension was stirred at 80° C. for 16 hours. After spontaneous cooling to room temperature, water was added, and an organic layer was separated. Sodium sulfate was added to the organic layer, and the resulting mixture was allowed to stand, after which the drying agent was filtered off, and the filtrate was concentrated under reduced pressure. The solid obtained was dissolved in chloroform, and the resulting solution was stirred with activated carbon added, after which Celite filtration was performed. The filtrate was concentrated, and then the solid obtained was purified by recrystallization from toluene to obtain 3,7-di[1-(4,4,5,5-tetramethyl-1,3,2-dioxaborolan-2-yl)phenyl-4-yl]diamantane as a white solid (1.21 g, 65% yield).

¹H-NMR (CDCl₃) δ (ppm): 7.79 (d, J=8.3 Hz, 4H), 7.43 (d, J=8.3 Hz, 4H), 1.96-1.98 (m, 18H), 1.34 (s, 24H).

In an argon atmosphere, 2-chloro-4,6-diphenylyl-1,3,5-triazine (62 mg, 0.23 mmol), 3,7-di[1-(4,4,5,5-tetramethyl-1,3,2-dioxaborolan-2-yl)phenyl-4-yl]diamantane (58 mg, 0.10 mmol) and tetrakis(triphenylphosphine) palladium (11 mg, 0.01 mmol) were dissolved in tetrahydrofuran (1 mL). A 5 M aqueous sodium hydroxide solution (0.12 mL, 0.58 mmol) was added thereto, and the resulting mixture was stirred at 80° C. for 15 hours. After spontaneous cooling to room temperature, methanol was added, and a precipitated solid was collected by filtration. The collected substance was dissolved in hot chlorobenzene, and the resulting solution was stirred with activated carbon added, after which Celite filtration was performed. The filtrate was concentrated and then purified by recrystallization from chlorobenzene to obtain compound (1-100) as a white solid (38 mg, 49% yield). No glass transition point was detected.

¹H-NMR (CDCl₃) δ (ppm): 8.78-8.80 (m, 8H), 8.74 (d, J=8.5 Hz, 4H), 7.57-7.65 (m, 16H), 2.08-2.10 (m, 18H).

### Synthesis Example-16

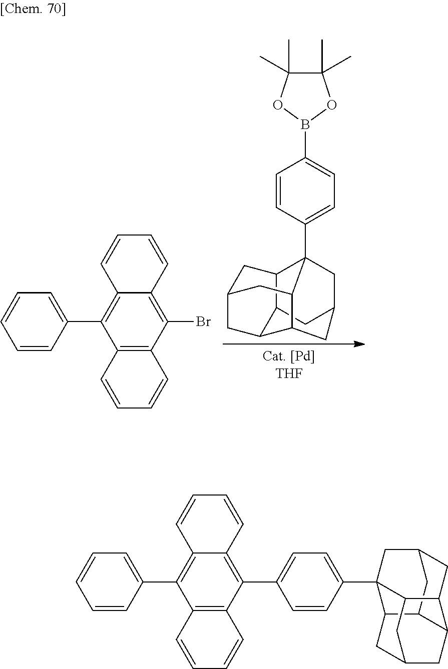

In an argon atmosphere, 4-(1-diamantyl)phenyl-4,4,5,5-tetramethyl-1,3,2-dioxaborolane (100 mg, 0.26 mmol), 9-bromo-10-phenylanthracene (102 mg, 0.31 mmol) and tetrakis(triphenylphosphine) palladium (9 mg, 0.01 mmol) were dissolved in tetrahydrofuran (2.6 mL). A 5 M aqueous sodium hydroxide solution (0.15 mL, 0.77 mmol) was added thereto, and the resulting mixture was stirred at 80° C. for 15 hours. After spontaneous cooling to room temperature, methanol was added, and a precipitated solid was collected by filtration. The collected substance was dissolved in hot toluene, and the resulting solution was stirred with activated carbon added, after which Celite filtration was performed. The filtrate was concentrated and then purified by recrystallization from toluene to obtain compound (1-122) as a white solid (112 mg, 84% yield). Its glass transition temperature was 140° C.

¹H-NMR (CDCl₃) δ (ppm): 7.75-7.78 (m, 2H), 7.67-7.70 (m, 2H), 7.55-7.62 (m, 5H), 7.48-7.50 (m, 2H), 7.42 (d, J=8.4 Hz, 2H), 7.30-7.36 (m, 4H), 2.49-2.51 (m, 2H), 2.06-2.09 (m, 4H), 1.97-2.00 (m, 1H), 1.79-1.89 (m, 6H), 1.75-1.77 (m, 4H), 1.53-1.58 (m, 1H).

### Synthesis Example-17

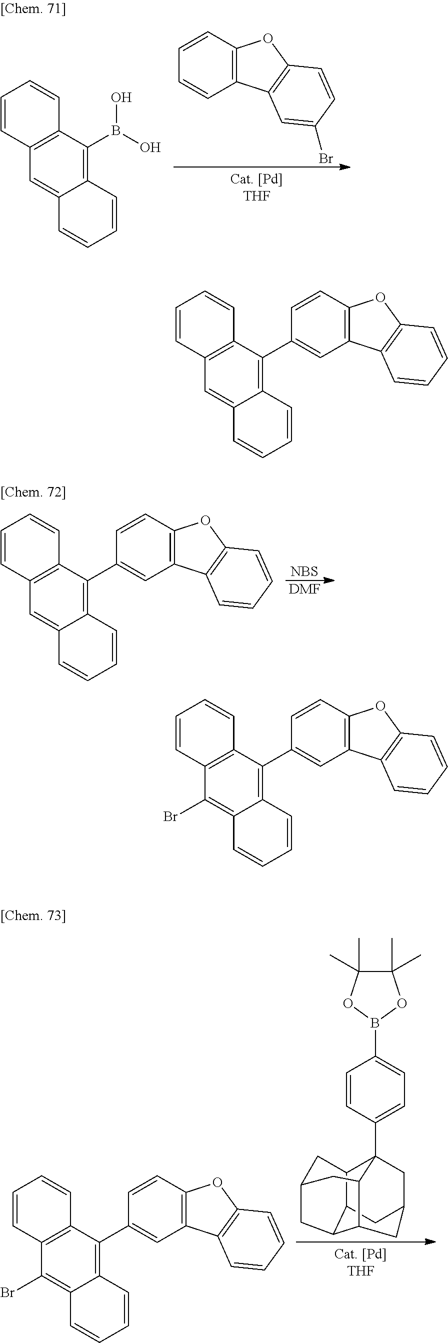

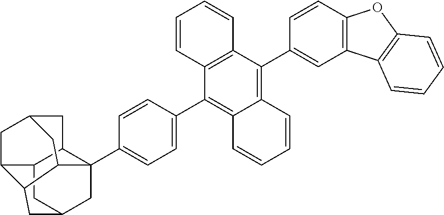 [SMILES: c1ccc2c(c1)oc1ccc(-c3c4ccccc4c(-c4ccc(C56CC7CC8C9CC(CC85)CC6C9C7)cc4)c4ccccc34)cc12]

In an argon atmosphere, 2-bromodibenzofuran (200 mg, 0.81 mmol), anthracene-9-boronic acid (216 mg, 0.97 mmol) and tetrakis(triphenylphosphine) palladium (47 mg, 0.04 mmol) were dissolved in tetrahydrofuran (8.1 mL). A 2 M aqueous potassium carbonate solution (1.21 mL, 2.43 mmol) was added thereto, and the resulting mixture was stirred at 80° C. for 15 hours. After spontaneous cooling to room temperature, water was added, and an organic layer was separated. Sodium sulfate was added to the organic layer, and the resulting mixture was allowed to stand, after which the drying agent was filtered off, and the filtrate was concentrated under reduced pressure. The crude product obtained was purified by silica gel column chromatography (hexane/toluene) to obtain 2-(9-anthracene) dibenzofuran as a yellow solid (223 mg, 80% yield).

¹H-NMR (CDCl₃) δ (ppm): 8.54 (s, 1H), 8.06-8.09 (m, 2H), 8.02-8.03 (m, 1H), 7.92-7.93 (m, 1H), 7.78 (dd, J=8.8, 0.5 Hz, 1H), 7.67-7.70 (m, 3H), 7.46-7.54 (m, 4H), 7.33-7.38 (m, 3H).

In an argon atmosphere, 2-(9-anthracene) dibenzofuran (100 mg, 0.29 mmol) was dissolved in dimethylformamide (1.0 mL). To this reaction solution, a solution obtained by dissolving N-bromosuccinimide (52 mg, 0.29 mmol) in dimethylformamide (1.3 mL) was added dropwise, and the resulting mixture was stirred at 60° C. for 3 hours. After spontaneous cooling to room temperature, water was added, and an organic layer was separated. Sodium sulfate was added to the organic layer, and the resulting mixture was allowed to stand, after which the drying agent was filtered off, and the filtrate was concentrated under reduced pressure. The crude product obtained was purified by silica gel column chromatography (hexane/toluene) to obtain 2-(10-bromoanthracen-9-yl) dibenzofuran as a yellow solid (108 mg, 88% yield).

¹H-NMR (CDCl₃) δ (ppm): 8.63-8.66 (m, 2H), 8.00 (dd, J=1.7, 0.5 Hz, 1H), 7.91-7.93 (m, 1H), 7.78 (dd, J=8.3, 0.5 Hz, 1H), 7.66-7.69 (m, 3H), 7.59-7.63 (m, 2H), 7.50-7.55 (m, 1H), 7.48 (dd, J=8.3, 1.7 Hz, 1H), 7.34-7.40 (m, 3H).

In an argon atmosphere, 4-(1-diamantyl)phenyl-4,4,5,5-tetramethyl-1,3,2-dioxaborolane (70 mg, 0.18 mmol), 2-(10-bromoanthracen-9-yl) dibenzofuran (91 mg, 0.22 mmol) and tetrakis triphenylphosphino palladium (6.2 mg, 0.01 mmol) were suspended in tetrahydrofuran (1.8 mL). A 5 M aqueous sodium hydroxide solution (0.11 mL, 0.54 mmol) was added to this suspension, and the resulting mixture was stirred at 80° C. for 16 hours. After spontaneous cooling to room temperature, water was added, and an organic layer was separated. Sodium sulfate was added to the organic layer, and the resulting mixture was allowed to stand, after which the drying agent was filtered off, and the filtrate was concentrated under reduced pressure. The crude product obtained was purified by silica gel column chromatography (hexane/toluene) to obtain compound (1-125) as a yellow solid (98 mg, 90% yield). Its glass transition temperature was 152° C.

¹H-NMR (CDCl₃) δ (ppm): 8.07-8.08 (m, 1H), 7.92-7.94 (m, 1H), 7.78-7.81 (m, 3H), 7.70-7.72 (m, 2H), 7.67-7.69 (m, 1H), 7.56-7.60 (m, 3H), 7.50-7.54 (m, 1H), 7.43-7.46 (m, 2H), 7.30-7.39 (m, 5H), 2.50-2.53 (m, 2H), 2.05-2.11 (m, 4H), 1.98-2.01 (m, 1H), 1.80-1.90 (m, 6H), 1.77-1.78 (m, 4H), 1.54-1.59 (m, 1H).

### Synthesis Example-18

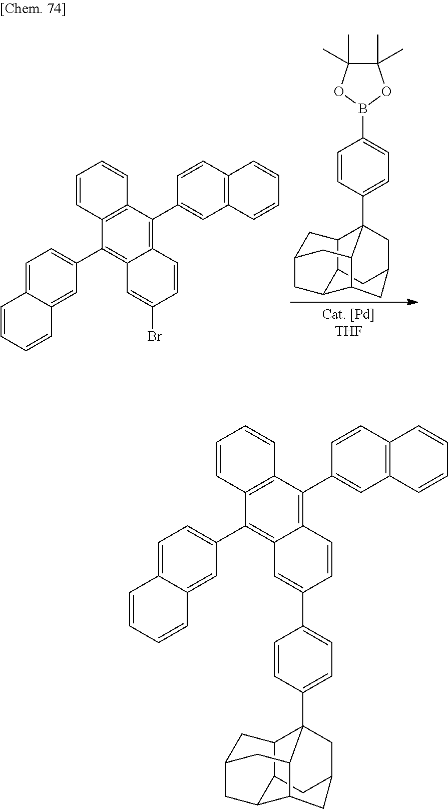

In an argon atmosphere, 4-(1-diamantyl)phenyl-4,4,5,5-tetramethyl-1,3,2-dioxaborolane (100 mg, 0.26 mmol), 2-bromo-9,10-di(1-naphthyl)anthracene (157 mg, 0.31 mmol) and tetrakis(triphenylphosphine) palladium (9 mg, 0.01 mmol) were dissolved in tetrahydrofuran (2.6 mL). A 5 M aqueous sodium hydroxide solution (0.15 mL, 0.77 mmol) was added thereto, and the resulting mixture was stirred at 80° C. for 15 hours. After spontaneous cooling to room temperature, methanol was added, and a precipitated solid was collected by filtration. The collected substance was dissolved in hot toluene, and the resulting solution was stirred with activated carbon added, after which Celite filtration was performed. The filtrate was concentrated and then purified by recrystallization from toluene to obtain compound (1-128) as a white solid (148 mg, 83% yield). No glass transition point was detected.

¹H-NMR (CDCl₃) δ (ppm): 8.11 (d, J=8.2 Hz, 2H), 8.03-8.06 (m, 4H), 7.93-7.96 (m, 3H), 7.81 (d, J=9.1 Hz, 1H), 7.66-7.76 (m, 4H), 7.60-7.64 (m, 5H), 7.47 (d, J=8.6 Hz, 2H), 7.29-7.33 (m, 4H), 2.29-2.32 (m, 2H), 1.94-1.97 (m, 2H), 1.83-1.87 (m, 1H), 1.81-1.83 (m, 1H), 1.73-1.80 (m, 6H), 1.60-1.66 (m, 3H), 1.50-1.51 (m, 2H), 1.37-1.42 (m, 2H).

### Synthesis Example-19

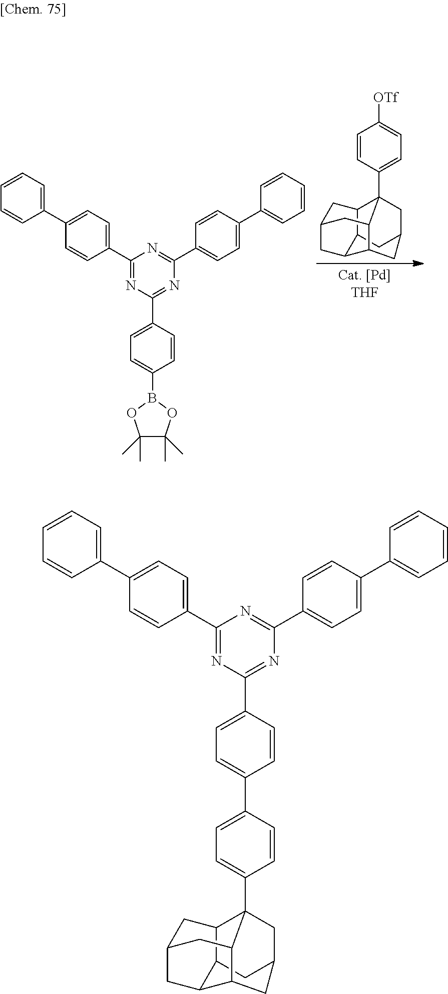

In an argon atmosphere, 2,4-bis([1,1′-biphenyl]-4-yl)-6-[4-(4,4,5,5-tetramethyl-1,3,2-dioxaborolan-2-yl)phenyl]-1,3,5-triazine (2.00 g, 3.40 mmol), 4-(1-diamantyl)phenyl trifluoromethanesulfonate (1.68 g, 4.08 mmol), palladium acetate (38 mg, 0.17 mmol) and 2-dicyclohexylphosphino-2′,4′,6′-triisopropylbiphenyl (0.16 g, 0.34 mol) were suspended in tetrahydrofuran (34 mL). A 2 M aqueous potassium carbonate solution (5.1 mL, 10.21 mmol) was added to this suspension, and the resulting mixture was stirred at 80° C. for 15 hours. After spontaneous cooling to room temperature, water and methanol were added, and a precipitated solid was collected by filtration. The collected substance was dissolved in chlorobenzene, and the resulting solution was stirred with activated carbon added, after which Celite filtration was performed. The filtrate was concentrated and then purified by recrystallization from toluene to obtain compound (1-28) as a white solid (yield amount 2.20 g, 89% yield). Its glass transition temperature was 166° C.

¹H-NMR (CDCl₃) δ (ppm): 8.86-8.89 (m, 6H), 7.85 (d, J=8.0 Hz, 2H), 7.83 (d, J=8.4 Hz, 4H), 7.72-7.74 (m, 4H), 7.70 (d, J=8.4 Hz, 2H), 7.48-7.53 (m, 6H), 7.47.45 (m, 2H), 2.41-2.45 (m, 2H), 2.01-2.04 (m, 2H), 1.89-1.93 (m, 3H), 1.78-1.85 (m, 5H), 1.70-1.73 (m, 3H), 1.61-1.63 (m, 2H), 1.57-1.52 (m, 2H).

### Synthesis Example-20

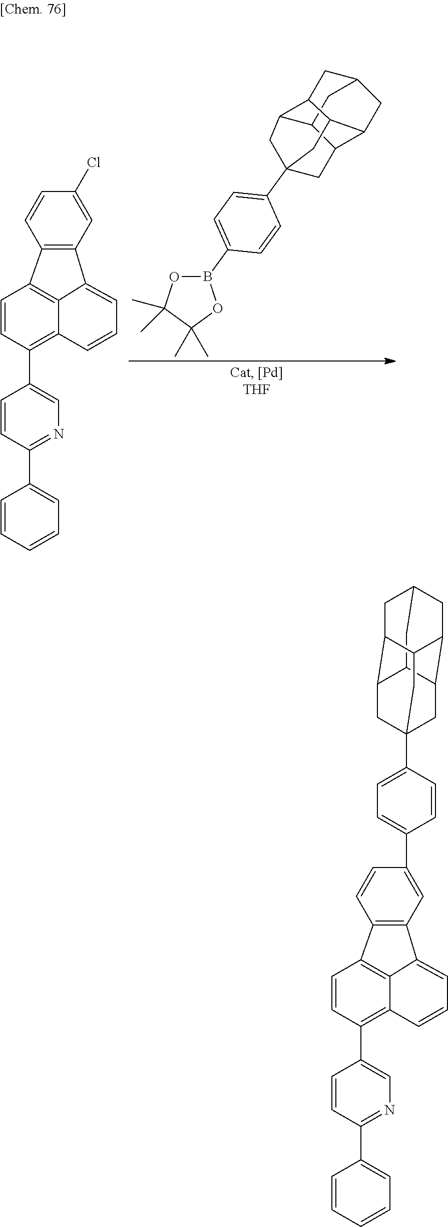

In an argon atmosphere, 5-(8-chloro-fluoranthen-3-yl)-2-phenyl-pyridine (1.00 g, 2.56 mmol), 4-(4-diamantyl)phenyl-4,4,5,5-tetramethyl-1,3,2-dioxaborolane (2.20 g, 5.64 mmol), palladium acetate (5.76 mg, 0.256 mmol) and tricyclohexylphosphine (24.5 mg, 0.513 mmol) were dissolved in tetrahydrofuran (26 mL). A 2 M aqueous potassium phosphate solution (3.85 mL, 7.69 mmol) was added thereto, and the resulting mixture was stirred at 70° C. for 5 hours. After spontaneous cooling to room temperature, a precipitated solid was collected by filtration. The collected substance was dissolved in hot toluene, and the resulting solution was stirred with activated carbon added, after which Celite filtration was performed. The filtrate was concentrated and then purified by recrystallization from toluene to obtain compound (1-130) as a white solid (1.52 g, 96% yield). Its glass transition temperature was 163° C.

¹H-NMR (CDCl₃) δ (ppm): 8.18 (d, J=2.4 Hz, 1H), 8.13 (d, J=1.2 Hz, 1H), 8.11 (brs, 1H), 8.08 (d, J=7.2 Hz, 1H), 8.01-8.02 (m, 1H), 8.00 (s, 1H), 7.97 (d, J=9.6 Hz, 1H), 7.93 (d, J=2.8 Hz, 1H), 7.64-7.72 (m, 6H), 7.52-7.56 (m, 5H), 1.95-1.98 (m, 10H), 1.92 (s, 1H), 1.78-1.82 (m, 9H).

### Synthesis Example-21

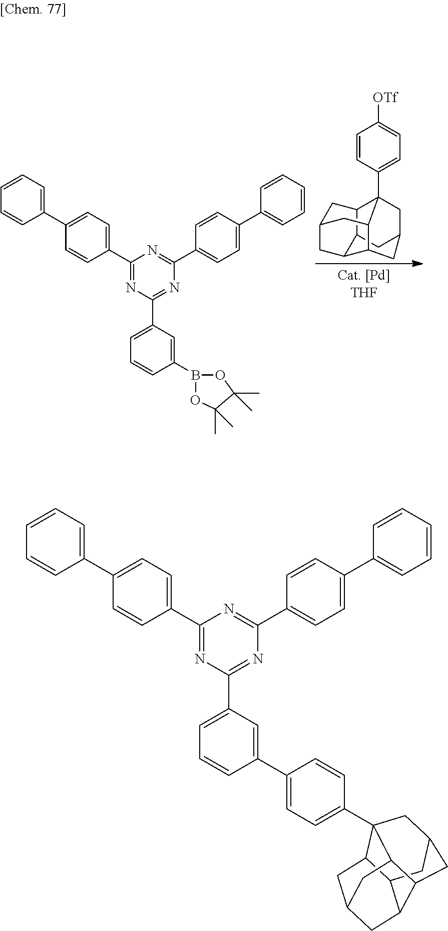

In an argon atmosphere, 2,4-bis([1,1′-biphenyl]-4-yl)-6-[3-(4,4,5,5-tetramethyl-1,3,2-dioxaborolan-2-yl)phenyl]-1,3,5-triazine (1.78 g, 3.18 mmol), 4-(1-diamantyl)phenyl trifluoromethanesulfonate (1.58 g, 3.82 mmol) and tetrakis(triphenylphosphine) palladium (308 mg, 0.32 mol) were suspended in tetrahydrofuran (30 mL). A 2 M aqueous tripotassium phosphate solution (4.8 mL, 9.54 mmol) was added to this suspension, and the resulting mixture was stirred at 80° C. for 15 hours. After spontaneous cooling to room temperature, water and methanol were added, and a precipitated solid was collected by filtration. The collected substance was dissolved in hot toluene, and the resulting solution was stirred with activated carbon added, after which Celite filtration was performed. The filtrate was concentrated and then purified by recrystallization from toluene to obtain compound (1-134) as a white solid (yield amount 1.40 g, 61% yield). Its glass transition temperature was 148° C.

¹H-NMR (CDCl₃) δ (ppm): 9.05 (t, J=1.6 Hz, 1H), 8.88 (d, J=8.6 Hz, 4H), 8.77 (dt, J=7.7, 1.6 Hz, 1H), 7.86-7.89 (m, 1H), 7.83 (d, J=8.6 Hz, 4H), 7.72-7.76 (m, 6H), 7.66 (t, J=7.7 Hz, 1H), 7.49-7.54 (m, 6H), 7.40-7.46 (m, 2H), 2.43-2.47 (m, 2H), 2.02-2.05 (m, 2H), 1.93-1.96 (m, 3H), 1.79-1.87 (m, 5H), 1.71-1.74 (m, 3H), 1.64-1.65 (m, 2H), 1.48-1.53 (m, 2H).

### Synthesis Example-22

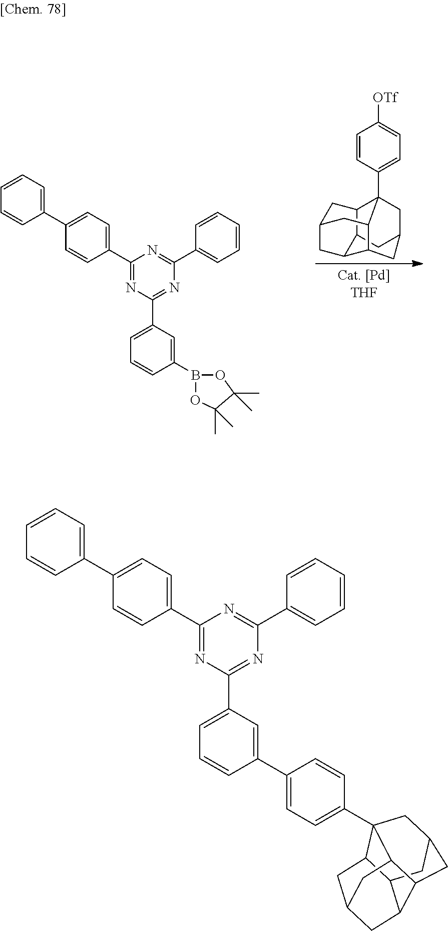 [SMILES: CC1(C)OB(c2cccc(-c3nc(-c4ccccc4)nc(-c4ccc(-c5ccccc5)cc4)n3)c2)OC1(C)C ; O=S(=O)(Oc1ccc(C23CC4CC5C6CC(CC52)CC3C6C4)cc1)C(F)(F)F ; c1ccc(-c2ccc(-c3nc(-c4ccccc4)nc(-c4cccc(-c5ccc(C67CC8CC9C%10CC(CC96)CC7C%10C8)cc5)c4)n3)cc2)cc1]

In an argon atmosphere, 2-([1,1′-biphenyl]-4-yl)-4-phenyl-6-[3-(4,4,5,5-tetramethyl-1,3,2-dioxaborolan-2-yl)phenyl]-1,3,5-triazine (1.58 g, 3.82 mmol), 4-(1-diamantyl)phenyl trifluoromethanesulfonate (1.66 g, 3.18 mmol) and tetrakis(triphenylphosphine) palladium (368 mg, 0.38 mol) were suspended in tetrahydrofuran (38 mL). A 2 M aqueous tripotassium phosphate solution (4.8 mL, 9.54 mmol) was added to this suspension, and the resulting mixture was stirred at 80° C. for 15 hours. After spontaneous cooling to room temperature, water and methanol were added, and a precipitated solid was collected by filtration. The collected substance was dissolved in hot toluene, and the resulting solution was stirred with activated carbon added, after which Celite filtration was performed. The filtrate was concentrated and then purified by recrystallization from toluene to obtain (1-135) as a white solid (yield amount 1.43 g, 58% yield). Its glass transition temperature was 133° C.

¹H-NMR (CDCl₃) δ (ppm): 9.05 (t, J=1.6 Hz, 1H), 8.88 (d, J=8.6 Hz, 2H), 8.81-8.84 (m, 2H), 8.75-8.78 (m, 1H), 7.86-7.88 (m, 1H), 7.83 (d, J=8.6 Hz, 2H), 7.72-7.75 (m, 4H), 7.59-7.67 (m, 4H), 7.50-7.54 (m, 4H), 7.40-7.45 (m, 1H), 2.43-2.46 (m, 2H), 2.02-2.05 (m, 1H), 1.93-1.96 (m, 3H), 1.79-1.88 (m, 5H), 1.71-1.74 (m, 3H), 1.65-1.66 (m, 2H), 1.48-1.53 (m, 2H).

### Synthesis Comparative Example-1

A compound represented by the following structural formula described in PTL 1 (also referred to herein as ETL-1) was synthesized according to a method described in PTL 1.

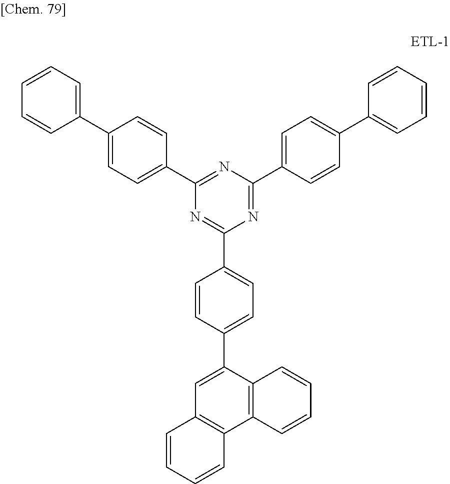 [SMILES: c1ccc(-c2ccc(-c3nc(-c4ccc(-c5ccccc5)cc4)nc(-c4ccc(-c5cc6ccccc6c6ccccc56)cc4)n3)cc2)cc1]

### <Production of Organic Electroluminescent Device>

Next, device evaluation was conducted using the diamantane compounds and ETL-1 obtained.

### Device Example-1 (See FIG. 2

### (Provision of Substrate 101 and Anode 102)

As a substrate having an anode on its surface, a glass substrate with an ITO transparent electrode, the glass substrate having a stripe pattern of 2 mm wide indium tin oxide (ITO) film (110 nm thick), was provided.

Subsequently, this substrate was washed with isopropyl alcohol and then surface treated by ozone ultraviolet cleaning.

### (Preparation for Vacuum Deposition)

On the cleaned substrate subjected to surface treatment, layers were vacuum deposited by a vacuum deposition method such that the layers were stacked on top of each other.

First, the glass substrate was introduced into a vacuum deposition chamber, and the pressure was reduced to 1.0×10⁻⁴ Pa. The layers were then formed in the following order according to their respective deposition conditions.

### (Formation of Hole Injection Layer 103)

N-[1,1′-Biphenyl]-4-yl-9,9-dimethyl-N-[4-(9-phenyl-9H-carbazol-3-yl)phenyl]-9H-fluorene-2-amine and 1,2,3-tris[(4-cyano-2,3,5,6-tetrafluorophenyl)methylene]cyclopropane subjected to sublimation purification were deposited by 10 nm at a rate of 0.15 nm/s to form a hole injection layer.

### (Formation of First Hole Transport Layer 1051)

N-[1,1′-Biphenyl]-4-yl-9,9-dimethyl-N-[4-(9-phenyl-9H-carbazol-3-yl)phenyl]-9H-fluorene-2-amine was deposited by 85 nm at a rate of 0.15 nm/s to form a first hole transport layer.

### (Formation of Second Hole Transport Layer 1052)

N-Phenyl-N-(9,9-diphenyl fluoren-2-yl)-N-(1,1′-biphenyl-4-yl)amine was deposited by 5 nm at a rate of 0.15 nm/s to form a second hole transport layer.

### (Formation of Light-Emitting Layer 106)

3-(10-Phenyl-9-anthryl)-dibenzofuran and 2,7-bis[N,N-di-(4-tert-butylphenyl)]amino-bisbenzofuranyl-9,9′-spirofluorene were deposited by 20 nm at a ratio of 95:5 (mass ratio) to form a light-emitting layer. The deposition rate was 0.18 nm/s.

### (Formation of First Electron Transport Layer 1071)

2-[3′-(9,9-Dimethyl-9H-fluoren-2-yl) [1,1′-biphenyl]-3-yl]-4,6-diphenyl-1,3,5-triazine (also referred to herein as ETL-2) subjected to sublimation purification was deposited by 6 nm at a rate of 0.15 nm/s to form a first electron transport layer.

### (Formation of Second Electron Transport Layer 1072)

(Compound (1-6) synthesized in Synthesis Example-1 and 8-hydroxyquinolinolato lithium (hereinafter referred to as Liq) were deposited by 25 nm at a ratio of 50:50 (mass ratio) to form a second electron transport layer. The deposition rate was 0.15 nm/s.

### (Formation of Cathode 108)

Finally, a metal mask was disposed orthogonally to the ITO stripes on the substrate, and a cathode 108 was deposited. The cathode was formed so as to have a three-layer structure in a manner that ytterbium, silver/magnesium (9/1, mass ratio) and silver were deposited in this order by 2 nm, 12 nm and 90 nm, respectively. The rate of deposition of ytterbium was 0.02 nm/s, the rate of deposition of silver/magnesium was 0.5 nm/s, and the rate of deposition of silver was 0.2 nm/s.

In the above manner, an organic electroluminescent device 100 having a light-emitting area of 4 mm² as shown in FIG. 2 was produced. The thickness of each layer was measured with a stylus thickness meter (DEKTAK, manufactured by Bruker).

Furthermore, this device was sealed in a nitrogen-atmosphere glove box with an oxygen and water concentration of 1 ppm or less. The sealing was performed using a bisphenol F epoxy resin (manufactured by Nagase ChemteX Corporation) with a glass sealing cap and the deposited substrate (device).

A direct current was applied to the organic electroluminescent device produced according to the above procedure, and its light-emission characteristics were evaluated using a luminance meter (product name: BM-9, manufactured by TOPCON TECHNOHOUSE CORPORATION). To evaluate the light-emission characteristics, current efficiency (cd/A) at a current density of 10 mA/cm² was measured.

The voltage and current efficiency are relative values based on reference values (100), which are the results in Device Comparative Example-1. The measurement results obtained are shown in Table 1.

### Device Example-2

An organic electroluminescent device was produced in the same manner as in Device Example-1 except that compound (1-27) synthesized in Synthesis Example-2 was used instead of compound (1-6), and evaluated. The measurement results obtained are shown in Table 1.

### Device Example-3

An organic electroluminescent device was produced in the same manner as in Device Example-1 except that compound (1-87) synthesized in Synthesis Example-3 was used instead of compound (1-6), and evaluated. The measurement results obtained are shown in Table 1.

### Device Example-4

An organic electroluminescent device was produced in the same manner as in Device Example-1 except that compound (1-19) synthesized in Synthesis Example-4 was used instead of compound (1-6), and evaluated. The measurement results obtained are shown in Table 1.

### Device Example-6

An organic electroluminescent device was produced in the same manner as in Device Example-1 except that compound (1-8) synthesized in Synthesis Example-6 was used instead of compound (1-6), and evaluated. The measurement results obtained are shown in Table 1.

### Device Example-7

An organic electroluminescent device was produced in the same manner as in Device Example-1 except that compound (1-9) synthesized in Synthesis Example-7 was used instead of compound (1-6), and evaluated. The measurement results obtained are shown in Table 1.

### Device Example-8

An organic electroluminescent device was produced in the same manner as in Device Example-1 except that compound (1-103) synthesized in Synthesis Example-8 was used instead of compound (1-6), and evaluated. The measurement results obtained are shown in Table 1.

### Device Example-9

An organic electroluminescent device was produced in the same manner as in Device Example-1 except that compound (1-104) synthesized in Synthesis Example-9 was used instead of compound (1-6), and evaluated. The measurement results obtained are shown in Table 1.

### Device Example-15

An organic electroluminescent device was produced in the same manner as in Device Example-1 except that compound (1-100) synthesized in Synthesis Example-15 was used instead of compound (1-6), and evaluated. The measurement results obtained are shown in Table 1.

### Device Example-18

An organic electroluminescent device was produced in the same manner as in Device Example-1 except that compound (1-128) synthesized in Synthesis Example-18 was used instead of compound (1-6), and evaluated. The measurement results obtained are shown in Table 1.

### Device Example-19

An organic electroluminescent device was produced in the same manner as in Device Example-1 except that compound (1-28) synthesized in Synthesis Example-19 was used instead of compound (1-6), and evaluated. The measurement results obtained are shown in Table 1.

### Device Example-20

An organic electroluminescent device was produced in the same manner as in Device Example-1 except that compound (1-130) synthesized in Synthesis Example-20 was used instead of compound (1-6), and evaluated. The measurement results obtained are shown in Table 1.

### Device Example-21

An organic electroluminescent device was produced in the same manner as in Device Example-1 except that compound (1-134) synthesized in Synthesis Example-21 was used instead of compound (1-6), and evaluated. The measurement results obtained are shown in Table 1.

### Device Example-22

An organic electroluminescent device was produced in the same manner as in Device Example-1 except that compound (1-135) synthesized in Synthesis Example-22 was used instead of compound (1-6), and evaluated. The measurement results obtained are shown in Table 1.

### Device Comparative Example-1

An organic electroluminescent device was produced in the same manner as in Device Example-1 except that ETL-1 was used instead of compound (1-6), and evaluated. The measurement results obtained are shown in Table 1.

TABLE 1

| | | | Current |
|---|---|---|---|
| | | Voltage | efficiency |
| | Compound | (V) | (cd/A) |
| Device Example-1 | Synthesis Example-1 | 99 | 101 |
| Device Example-2 | Synthesis Example-2 | 95 | 105 |
| Device Example-3 | Synthesis Example-3 | 93 | 102 |
| Device Example-4 | Synthesis Example-4 | 93 | 101 |
| Device Example-6 | Synthesis Example-6 | 98 | 105 |
| Device Example-7 | Synthesis Example-7 | 99 | 101 |
| Device Example-8 | Synthesis Example-8 | 94 | 105 |
| Device Example-9 | Synthesis Example-9 | 94 | 103 |
| Device Example-15 | Synthesis Example-15 | 94 | 109 |
| Device Example-18 | Synthesis Example-18 | 99 | 102 |
| Device Example-19 | Synthesis Example-19 | 96 | 104 |
| Device Example-20 | Synthesis Example-20 | 97 | 101 |
| Device Example-21 | Synthesis Example-21 | 98 | 108 |
| Device Example-22 | Synthesis Example-22 | 99 | 109 |
| Device Comparative | ETL-1 | 100 | 100 |
| Example-1 | | | |

### Device Example-5 (See FIG. 2)

### (Provision of Substrate 101 and Anode 102)

As a substrate having an anode on its surface, a glass substrate with an ITO transparent electrode, the glass substrate having a stripe pattern of 2 mm wide indium tin oxide (ITO) film (110 nm thick), was provided.

Subsequently, this substrate was washed with isopropyl alcohol and then surface treated by ozone ultraviolet cleaning.

### (Preparation for Vacuum Deposition)

On the cleaned substrate subjected to surface treatment, layers were vacuum deposited by a vacuum deposition method such that the layers were stacked on top of each other.

First, the glass substrate was introduced into a vacuum deposition chamber, and the pressure was reduced to 1.0×10⁻⁴ Pa. The layers were then formed in the following order according to their respective deposition conditions.

### (Formation of Hole Injection Layer 103)

N-[1,1′-Biphenyl]-4-yl-9,9-dimethyl-N-[4-(9-phenyl-9H-carbazol-3-yl)phenyl]-9H-fluorene-2-amine and 1,2,3-tris[(4-cyano-2,3,5,6-tetrafluorophenyl)methylene]cyclopropane subjected to sublimation purification were deposited by 10 nm at a rate of 0.15 nm/s to form a hole injection layer.

### (Formation of First Hole Transport Layer 1051)

N-[1,1′-Biphenyl]-4-yl-9,9-dimethyl-N-[4-(9-phenyl-9H-carbazol-3-yl)phenyl]-9H-fluorene-2-amine was deposited by 85 nm at a rate of 0.15 nm/s to form a first hole transport layer.

### (Formation of Second Hole Transport Layer 1052)

N-Phenyl-N-(9,9-diphenyl fluoren-2-yl)-N-(1,1′-biphenyl-4-yl)amine was deposited by 5 nm at a rate of 0.15 nm/s to form a second hole transport layer.

### (Formation of Light-Emitting Layer 106)

3-(10-Phenyl-9-anthryl)-dibenzofuran and 2,7-bis[N,N-di-(4-tert-butylphenyl)]amino-bisbenzofuranyl-9,9′-spirofluorene were deposited by 20 nm at a ratio of 95:5 (mass ratio) to form a light-emitting layer. The deposition rate was 0.18 nm/s.

### (Formation of First Electron Transport Layer 1071)

(Compound 1-54) synthesized in Synthesis Example-5 was deposited by 6 nm at a rate of 0.15 nm/s to form a first electron transport layer.

### (Formation of Second Electron Transport Layer 1072)

4,6-Diphenyl-2-(4-{4-[4′-cyano-(1,1′-biphenyl)-4-yl]naphthalen-1-yl}phenyl)-1,3,5-triazine and 8-hydroxyquinolinolato lithium (hereinafter referred to as Liq) subjected to sublimation purification were deposited by 25 nm at a ratio of 50:50 (mass ratio) to form a second electron transport layer. The deposition rate was 0.15 nm/s.

### (Formation of Cathode 108)

Finally, a metal mask was disposed orthogonally to the ITO stripes on the substrate, and a cathode 108 was deposited. The cathode was formed so as to have a three-layer structure in a manner that ytterbium, silver/magnesium (9/1, mass ratio) and silver were deposited in this order by 2 nm, 12 nm and 90 nm, respectively. The rate of deposition of ytterbium was 0.02 nm/s, the rate of deposition of silver/magnesium was 0.5 nm/s, and the rate of deposition of silver was 0.2 nm/s.

In the above manner, an organic electroluminescent device 100 having a light-emitting area of 4 mm² as shown in FIG. 2 was produced. The thickness of each layer was measured with a stylus thickness meter (DEKTAK, manufactured by Bruker).

Furthermore, this device was sealed in a nitrogen-atmosphere glove box with an oxygen and water concentration of 1 ppm or less. The sealing was performed using a bisphenol F epoxy resin (manufactured by Nagase ChemteX Corporation) with a glass sealing cap and the deposited substrate (device).

A direct current was applied to the organic electroluminescent device produced according to the above procedure, and its light-emission characteristics were evaluated using a luminance meter (product name: BM-9, manufactured by TOPCON TECHNOHOUSE CORPORATION). To evaluate the light-emission characteristics, current efficiency (cd/A) at a current density of 10 mA/cm² was measured.

The voltage and current efficiency are relative values based on reference values (100), which are the results in Device Comparative Example-2. The measurement results obtained are shown in Table 2.

### Device Comparative Example-2

An organic electroluminescent device was produced in the same manner as in Device Example-5 except that a compound (ETL-2) described in Synthesis Example 11 of PTL 2 was used instead of compound (1-54), and evaluated. The measurement results obtained are shown in Table 2.

TABLE 2

| | | | Current |
|---|---|---|---|
| | | Voltage | efficiency |
| | Compound | (V) | (cd/A) |
| Device Example-5 | Synthesis Example-5 | 97 | 111 |
| Device Comparative | ETL-2 | 100 | 100 |
| Example-2 | | | |

### Device Example-10 (See FIG. 2)

### (Provision of Substrate 101 and Anode 102)

As a substrate having an anode on its surface, a glass substrate with an ITO transparent electrode, the glass substrate having a stripe pattern of 2 mm wide indium tin oxide (ITO) film (110 nm thick), was provided.

Subsequently, this substrate was washed with isopropyl alcohol and then surface treated by ozone ultraviolet cleaning.

### (Preparation for Vacuum Deposition)

On the cleaned substrate subjected to surface treatment, layers were vacuum deposited by a vacuum deposition method such that the layers were stacked on top of each other.

First, the glass substrate was introduced into a vacuum deposition chamber, and the pressure was reduced to 1.0×10⁻⁴ Pa. The layers were then formed in the following order according to their respective deposition conditions.

### (Formation of Hole Injection Layer 103)

N-[1,1′-Biphenyl]-4-yl-9,9-dimethyl-N-[4-(9-phenyl-9H-carbazol-3-yl)phenyl]-9H-fluorene-2-amine and 1,2,3-tris[(4-cyano-2,3,5,6-tetrafluorophenyl)methylene]cyclopropane subjected to sublimation purification were deposited by 10 nm at a rate of 0.15 nm/s to form a hole injection layer.

### (Formation of First Hole Transport Layer 1051)

(Compound (1-106) synthesized in Synthesis Example-10 was deposited by 85 nm at a rate of 0.15 nm/s to form a first hole transport layer.

### (Formation of Second Hole Transport Layer 1052)

N-Phenyl-N-(9,9-diphenyl fluoren-2-yl)-N-(1,1′-biphenyl-4-yl)amine was deposited by 5 nm at a rate of 0.15 nm/s to form a second hole transport layer.

### (Formation of Light-Emitting Layer 106)

3-(10-Phenyl-9-anthryl)-dibenzofuran and 2,7-bis[N,N-di-(4-tert-butylphenyl)]amino-bisbenzofurano-9,9′-spirofluorene were deposited by 20 nm at a ratio of 95:5 (mass ratio) to form a light-emitting layer. The deposition rate was 0.18 nm/s.

### (Formation of First Electron Transport Layer 1071)

2-[3′-(9,9-Dimethyl-9H-fluoren-2-yl) [1,1′-biphenyl]-3-yl]-4,6-diphenyl-1,3,5-triazine subjected to sublimation purification was deposited by 6 nm at a rate of 0.15 nm/s to form a first electron transport layer.

### (Formation of Second Electron Transport Layer 1072)

4,6-Diphenyl-2-(4-{4-[4′-cyano-(1,1′-biphenyl)-4-yl]naphthalen-1-yl}phenyl)-1,3,5-triazine and 8-hydroxyquinolinolato lithium (hereinafter referred to as Liq) subjected to sublimation purification were deposited by 25 nm at a ratio of 50:50 (mass ratio) to form a second electron transport layer. The deposition rate was 0.15 nm/s.

### (Formation of Cathode 108)

Finally, a metal mask was disposed orthogonally to the ITO stripes on the substrate, and a cathode 108 was deposited. The cathode was formed so as to have a three-layer structure in a manner that ytterbium, silver/magnesium (9/1, mass ratio) and silver were deposited in this order by 2 nm, 12 nm and 90 nm, respectively. The rate of deposition of ytterbium was 0.02 nm/s, the rate of deposition of silver/magnesium was 0.5 nm/s, and the rate of deposition of silver was 0.2 nm/s.

In the above manner, an organic electroluminescent device 100 having a light-emitting area of 4 mm² as shown in FIG. 2 was produced. The thickness of each layer was measured with a stylus thickness meter (DEKTAK, manufactured by Bruker).

Furthermore, this device was sealed in a nitrogen-atmosphere glove box with an oxygen and water concentration of 1 ppm or less. The sealing was performed using a bisphenol F epoxy resin (manufactured by Nagase ChemteX Corporation) with a glass sealing cap and the deposited substrate (device).

A direct current was applied to the organic electroluminescent device produced according to the above procedure, and its light-emission characteristics were evaluated using a luminance meter (product name: BM-9, manufactured by TOPCON TECHNOHOUSE CORPORATION). To evaluate the light-emission characteristics, current efficiency (cd/A) at a current density of 10 mA/cm² was measured.

The current efficiency is a relative value based on a reference value (100), which is the result in Device Comparative Example-3. The measurement results obtained are shown in Table 3.

### Device Example-11

An organic electroluminescent device was produced in the same manner as in Device Example-10 except that compound (1-113) synthesized in Synthesis Example-11 was used instead of compound (1-106), and evaluated. The measurement results obtained are shown in Table 3.

### Device Example-12

An organic electroluminescent device was produced in the same manner as in Device Example-10 except that compound (1-108) synthesized in Synthesis Example-12 was used instead of compound (1-106), and evaluated. The measurement results obtained are shown in Table 3.

### Device Example-13

An organic electroluminescent device was produced in the same manner as in Device Example-10 except that compound (1-117) synthesized in Synthesis Example-13 was used instead of compound (1-106), and evaluated. The measurement results obtained are shown in Table 3.

### Device Example-14

An organic electroluminescent device was produced in the same manner as in Device Example-10 except that compound (1-118) synthesized in Synthesis Example-14 was used instead of compound (1-106), and evaluated. The measurement results obtained are shown in Table 3.

### Device Comparative Example-3

An organic electroluminescent device was produced in the same manner as in Device Example-10 except that N-[1,1′-biphenyl]-4-yl-9,9-dimethyl-N-[4-(9-phenyl-9H-carbazol-3-yl)phenyl]-9H-fluorene-2-amine (HTL-1) was used instead of compound (1-106), and evaluated. The measurement results obtained are shown in Table 3.

TABLE 3

| | | Current |
|---|---|---|
| | Compound | efficiency (cd/A) |
| Device Example-10 | Synthesis Example-10 | 104 |
| Device Example-11 | Synthesis Example-11 | 109 |
| Device Example-12 | Synthesis Example-12 | 106 |
| Device Example-13 | Synthesis Example-13 | 104 |
| Device Example-14 | Synthesis Example-14 | 103 |
| Device Comparative | HTL-1 | 100 |
| Example-3 | | |

### Device Example-16 (See FIG. 2)

### (Provision of Substrate 101 and Anode 102)

As a substrate having an anode on its surface, a glass substrate with an ITO transparent electrode, the glass substrate having a stripe pattern of 2 mm wide indium tin oxide (ITO) film (110 nm thick), was provided. Subsequently, this substrate was washed with isopropyl alcohol and then surface treated by ozone ultraviolet cleaning.

### (Preparation for Vacuum Deposition)

On the cleaned substrate subjected to surface treatment, layers were vacuum deposited by a vacuum deposition method such that the layers were stacked on top of each other.

First, the glass substrate was introduced into a vacuum deposition chamber, and the pressure was reduced to 1.0×10⁻⁴ Pa. The layers were then formed in the following order according to their respective deposition conditions.

### (Formation of Hole Injection Layer 103)

N-[1,1′-Biphenyl]-4-yl-9,9-dimethyl-N-[4-(9-phenyl-9H-carbazol-3-yl)phenyl]-9H-fluorene-2-amine and 1,2,3-tris[(4-cyano-2,3,5,6-tetrafluorophenyl)methylene]cyclopropane subjected to sublimation purification were deposited by 10 nm at a rate of 0.15 nm/s to form a hole injection layer.

### (Formation of First Hole Transport Layer 1051)

(Compound (1-106) synthesized in Synthesis Example-10 was deposited by 85 nm at a rate of 0.15 nm/s to form a first hole transport layer.

### (Formation of Second Hole Transport Layer 1052)

N-Phenyl-N-(9,9-diphenyl fluoren-2-yl)-N-(1,1′-biphenyl-4-yl)amine was deposited by 5 nm at a rate of 0.15 nm/s to form a second hole transport layer.

### (Formation of Light-Emitting Layer 106)

Compound (1-122) synthesized in Synthesis Example-16 and 2,7-bis[N,N-di-(4-tert-butylphenyl)]amino-bisbenzofurano-9,9′-spirofluorene were deposited by 20 nm at a ratio of 95:5 (mass ratio) to form a light-emitting layer. The deposition rate was 0.18 nm/s.

### (Formation of First Electron Transport Layer 1071)

2-[3′-(9,9-Dimethyl-9H-fluoren-2-yl) [1,1′-biphenyl]-3-yl]-4,6-diphenyl-1,3,5-triazine subjected to sublimation purification was deposited by 6 nm at a rate of 0.15 nm/s to form a first electron transport layer.

### (Formation of Second Electron Transport Layer 1072)

4,6-Diphenyl-2-(4-{4-[4′-cyano-(1,1′-biphenyl)-4-yl]naphthalen-1-yl}phenyl)-1,3,5-triazine and 8-hydroxyquinolinolato lithium (hereinafter referred to as Liq) subjected to sublimation purification were deposited by 25 nm at a ratio of 50:50 (mass ratio) to form a second electron transport layer. The deposition rate was 0.15 nm/s.

### (Formation of Cathode 108)

Finally, a metal mask was disposed orthogonally to the ITO stripes on the substrate, and a cathode 108 was deposited. The cathode was formed so as to have a three-layer structure in a manner that ytterbium, silver/magnesium (9/1, mass ratio) and silver were deposited in this order by 2 nm, 12 nm and 90 nm, respectively. The rate of deposition of ytterbium was 0.02 nm/s, the rate of deposition of silver/magnesium was 0.5 nm/s, and the rate of deposition of silver was 0.2 nm/s.

In the above manner, an organic electroluminescent device 100 having a light-emitting area of 4 mm² as shown in FIG. 2 was produced. The thickness of each layer was measured with a stylus thickness meter (DEKTAK, manufactured by Bruker).

Furthermore, this device was sealed in a nitrogen-atmosphere glove box with an oxygen and water concentration of 1 ppm or less. The sealing was performed using a bisphenol F epoxy resin (manufactured by Nagase ChemteX Corporation) with a glass sealing cap and the deposited substrate (device).

A direct current was applied to the organic electroluminescent device produced according to the above procedure, and its light-emission characteristics were evaluated using a luminance meter (product name: BM-9, manufactured by TOPCON TECHNOHOUSE CORPORATION). To evaluate the light-emission characteristics, current efficiency (cd/A) at a current density of 10 mA/cm² was measured.

The current efficiency is a relative value based on a reference value (100), which is the result in Device Comparative Example-4. The measurement results obtained are shown in Table 4.

### Device Example-17

An organic electroluminescent device was produced in the same manner as in Device Example-16 except that compound (1-125) synthesized in Synthesis Example-17 was used instead of compound (1-122), and evaluated. The measurement results obtained are shown in Table 4.

### Device Comparative Example-4

An organic electroluminescent device was produced in the same manner as in Device Example-16 except that 3-(10-phenyl-9-anthryl)-dibenzofuran (EML-1) was used instead of compound (1-122), and evaluated. The measurement results obtained are shown in Table 4.

TABLE 4

| | | Current |
|---|---|---|
| | Compound | efficiency (cd/A) |
| Device Example-16 | Synthesis Example-16 | 102 |
| Device Example-17 | Synthesis Example-17 | 101 |
| Device Comparative | EML-1 | 100 |
| Example-4 | | |

Using the diamantane compound according to an aspect of the present invention can provide an organic electroluminescent device excellent in luminous efficiency, more preferably excellent in drive voltage and luminous efficiency.

The diamantane compound according to an aspect of the present invention can be used in an organic electroluminescent device material (specifically, an electron transport material for an organic electroluminescent device, a hole transport material for an organic electroluminescent device, etc.) conducive to production of an organic electroluminescent device excellent in luminous efficiency, more preferably excellent in low drive voltage and luminous efficiency. Furthermore, the diamantane compound according to an aspect of the present invention can provide an organic electroluminescent device with high luminous efficiency, more preferably an organic electroluminescent device with low power consumption and high luminous efficiency.

### REFERENCE SIGNS LIST

100. organic electroluminescent device
1. substrate
2. anode
3. hole injection layer
4. hole transport layer
5. light-emitting layer
6. electron transport layer
7. electron injection layer
8. cathode
101. substrate
102. anode
103. hole injection layer
105. hole transport layer
1051. first hole transport layer
1052. second hole transport layer
106. light-emitting layer
107. electron transport layer
1071. first electron transport layer
1072. second electron transport layer
108. cathode

1. A diamantane compound comprising a group represented by formula (1) below and a group represented by formula (2) below, 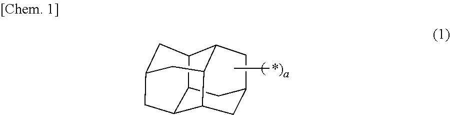
(in formula (1), carbon atoms of a diamantane ring are optionally substituted with an aryl group having 6 to 12 carbon atoms, and in formula (1), * represents a bond, and a represents an integer of 1 to 6), 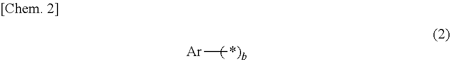
(Ar in formula (2) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
provided that in Ar, a total number of carbon atoms constituting Ar is 8 or more, and when Ar is constituted only by the aromatic hydrocarbon group having 6 to 60 carbon atoms of (i), the total number of carbon atoms constituting Ar is 13 or more, and in formula (2), * represents a bond, and b represents an integer of 1 to 6).

2. The diamantane compound according to claim 1, wherein the diamantane compound is represented by formula (3) below or formula (4) below, 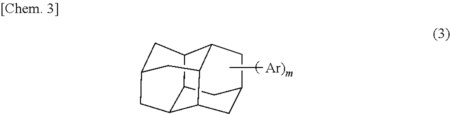
(in a diamantane ring in formula (3), carbon atoms other than a carbon atom bonded to an —Ar group are optionally substituted with an aryl group having 6 to 12 carbon atoms,
each Ar in formula (3) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
provided that in each independent Ar in formula (3), a total number of carbon atoms constituting Ar is 8 or more, and when Ar is constituted only by the aromatic hydrocarbon group having 6 to 60 carbon atoms of (i), the total number of carbon atoms constituting Ar is 13 or more, and
in formula (3), m represents an integer of 1, 2 or 3), 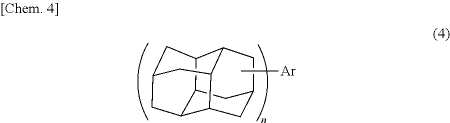
(in each independent diamantane ring in formula (4), carbon atoms other than a carbon atom bonded to an —Ar group are optionally substituted with an aryl group having 6 to 12 carbon atoms,
Ar in formula (4) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
provided that in Ar in formula (4), a total number of carbon atoms constituting Ar is 8 or more, and when Ar is constituted only by the aromatic hydrocarbon group having 6 to 60 carbon atoms of (i), the total number of carbon atoms constituting Ar is 13 or more, and
in formula (4), n represents an integer of 2 or 3).

3. The diamantane compound according to claim 2, wherein the diamantane compound is represented by any of formula (3-1) below to formula (3-3) below, 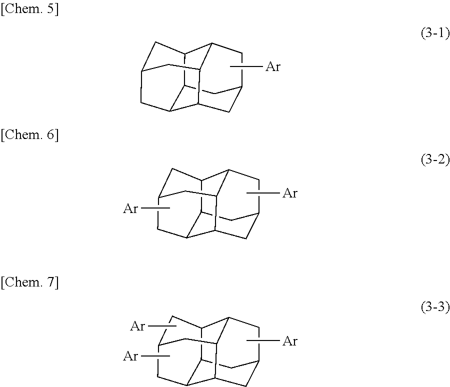
(in diamantane rings in formula (3-1) to formula (3-3), carbon atoms other than a carbon atom bonded to an —Ar group are optionally substituted with an aryl group having 6 to 12 carbon atoms,
each Ar in formula (3-1) to formula (3-3) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
provided that in each independent Ar in formula (3-1) to formula (3-3), a total number of carbon atoms constituting Ar is 8 or more, and when Ar is constituted only by the aromatic hydrocarbon group having 6 to 60 carbon atoms of (i), the total number of carbon atoms constituting Ar is 13 or more).

4. The diamantane compound according to claim 2, wherein the diamantane compound is represented by formula (4-1) below or formula (4-2) below, 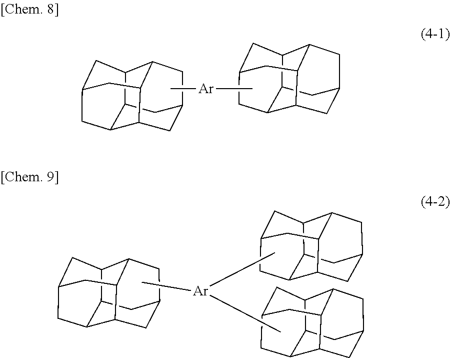
(in each independent diamantane ring in formula (4-1) and formula (4-2), carbon atoms other than a carbon atom bonded to an —Ar group are optionally substituted with an aryl group having 6 to 12 carbon atoms,
Ar in formula (4-1) and formula (4-2) represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
provided that in Ar in formula (4-1) and formula (4-2), a total number of carbon atoms constituting Ar is 8 or more, and when Ar is constituted only by the aromatic hydrocarbon group having 6 to 60 carbon atoms of (i), the total number of carbon atoms constituting Ar is 13 or more.

5. The diamantane compound according to claim 1, wherein the heteroaromatic group is a monocyclic ring or fused ring including any of a nitrogen atom, an oxygen atom and a sulfur atom, or a linked ring formed by linkage of any rings selected from such rings.

6. The diamantane compound according to claim 1, wherein the aromatic hydrocarbon group includes an arylamino group, or
the heteroaromatic group includes a heterocyclic amino group.

7. The diamantane compound according to claim 1, wherein a substituent in the optionally substituted aromatic hydrocarbon group of (i) or the optionally substituted heteroaromatic group of (ii) is a group selected from the group consisting of a fluorine atom, a chlorine atom, a bromine atom, an iodine atom, a formyl group, a cyano group, a nitro group, alkyl groups having 1 to 20 carbon atoms, alkenyl groups having 1 to 20 carbon atoms, cycloalkyl groups having 1 to 20 carbon atoms, bicycloalkyl groups having 1 to 20 carbon atoms, tricycloalkyl groups having 1 to 20 carbon atoms, alkoxy groups having 1 to 10 carbon atoms, P(═O)(Ar²)₂, C(═O)Ar², B(Ar²)₂, B(OAr²)₂, OSO₂Ar², S(═O)Ar², Si(Ar²)₃, P(═S)(Ar²)₂ and C(═C(CN)₂)Ar²₂, (where Ar² is a group selected from the group consisting of alkyl groups having 1 to 10 carbon atoms, aryl groups having 6 to 60 carbon atoms and heteroaromatic groups having 3 to 60 carbon atoms).

8. The diamantane compound according to claim 5, wherein the diamantane compound has the optionally substituted heteroaromatic group having 3 to 60 carbon atoms of (ii), the heteroaromatic group including any of a nitrogen atom, an oxygen atom and a sulfur atom.

9. The diamantane compound according to claim 8, wherein the diamantane compound has the optionally substituted heteroaromatic group having 3 to 60 carbon atoms of (ii), the heteroaromatic group including a nitrogen atom.

10. The diamantane compound according to claim 1, wherein the diamantane compound has a fused-ring aromatic hydrocarbon group having 14 or more carbon atoms.

11. The diamantane compound according to claim 1, wherein in the diamantane ring, carbon atoms other than a carbon atom bonded to an —Ar group are not substituted with an aryl group.

12. An organic electroluminescent device material comprising the diamantane compound according to claim 1.

13. The organic electroluminescent device material according to claim 12, wherein the organic electroluminescent device material is an electron transport material for an organic electroluminescent device or a hole transport material for an organic electroluminescent device.

14. An organic electroluminescent device comprising the diamantane compound according to claim 1.

15. A diamantane compound represented by formula (5) below or formula (6) below, 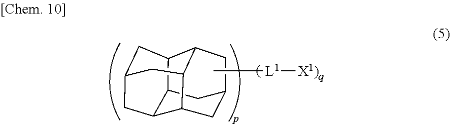
(in a diamantane ring in formula (5), carbon atoms other than a carbon atom bonded to an -L¹-X¹ group are optionally substituted with an aryl group having 6 to 12 carbon atoms,
each L¹ in formula (5) independently represents
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
a total number of carbon atoms constituting L¹ is 8 or more,
X¹ in formula (5) represents a halogen atom or a trifluoromethanesulfonyloxy group, and
in formula (5), p and q each represent an integer of 1 to 3, when p is 1, q represents an integer of 1, 2 or 3, and when q is 1, p represents an integer of 1, 2 or 3), 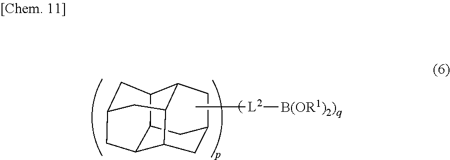
(in a diamantane ring in formula (6), carbon atoms other than a carbon atom bonded to an -L²-B(OR¹)₂ group are optionally substituted with an aryl group having 6 to 12 carbon atoms,
each L² in formula (6) independently represents a single bond or
(i) an optionally substituted aromatic hydrocarbon group having 6 to 60 carbon atoms,
(ii) an optionally substituted heteroaromatic group having 3 to 60 carbon atoms or
(iii) a group that is a combination of the groups of (i) and (ii),
R¹ in formula (6) represents a hydrogen atom, an alkyl group having 1 to 4 carbon atoms or a phenyl group, two R¹'s of B(OR¹)₂ may be the same or different, the two R¹'s may be combined to form a ring including an oxygen atom and a boron atom, and
in formula (6), p and q each represent an integer of 1 to 3, when p is 1, q represents an integer of 1, 2 or 3, and when q is 1, p represents an integer of 1, 2 or 3).
